(19) United States
(12) Patent Application Publication
(10) Pub. No.: US 2009/0221488 A1
(43) Pub. Date: Sep. 3, 2009

(54) Certain Compositions and Methods of Treatment

(76) Inventors: Kenneth W. Wood, Foster City, CA (US); Lisa Belmont, Berkeley, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/096,170

(22) PCT Filed: Dec. 7, 2006
(86) PCT No.: PCT/US2006/046913

§ 371 Date: Oct. 24, 2008

Related U.S. Application Data
(60) Provisional application No. 60/748,753, filed on Dec. 8, 2005.
(60) Provisional application No. 60/817,976, filed on Jun. 29, 2006.

(51) Int. Cl. A61K 38/16; A61K 31/69; A61K 31/352; A61K 31/4164; A61K 31/357; A61K 31/4155; A61K 31/381; A61K 31/42; A61K 31/423; A61K 31/4025; A61P 35/00

(52) U.S. Cl. 514/12; 514/64; 514/456; 514/397; 514/452; 514/406; 514/444; 514/378; 514/379; 514/422

(57) ABSTRACT

Disclosed inter alia is the use of certain chromenone derivatives, which are modulators of a mitotic kinesin such as KSP, in the treatment of cellular proliferative diseases. The chromenones derivatives are administered with another chemotherapeutic agent selected from neutropenia treatment agents, alkylating agents, antimetabolites, platinating agents, topoisomerase inhibitors, tubulin agents and signalling inhibitors (e.g., kinase inhibitors). Pharmaceutical compositions comprising one or both types of active agents are also disclosed.

This application claims the benefit of U.S. Provisional Patent Application No. 60/748,753, filed Dec. 8, 2005, and U.S. Provisional Patent Application No. 60/817,976, filed Jun. 29, 2006; each of which is incorporated herein by reference for all purposes.

Provided are certain chromenone derivatives which are modulators of a mitotic kinesin, particularly the mitotic kinesin KSP. In particular, provided is the use of such derivatives in the treatment of cellular proliferative diseases such as cancer, hyperplasias, restenosis, cardiac hypertrophy, immune disorders and inflammation.

The mitotic spindle has been an important target in cancer chemotherapy as demonstrated by the anti-tubulin agents vincristine, vinblastine and vinorelbine. E.g., see Wood et al., “Past and Future of the Mitotic Spindle as an Oncology Target.” Current Opinion in Pharmacology, 2001, 1, 370-377, which is hereby incorporated by reference in its entirety.

Taxanes and vinca alkaloids act on microtubules, which are present in a variety of cellular structures. Microtubules are the primary structural element of the mitotic spindle. The mitotic spindle is responsible for distribution of replicate copies of the genome to each of the two daughter cells that result from cell division. It is presumed that disruption of the mitotic spindle by these drugs results in inhibition of cancer cell division, and induction of cancer cell death. However, microtubules form other types of cellular structures, including tracks for intracellular transport in nerve processes. Because these agents do not specifically target mitotic spindles, they have side effects that limit their usefulness.

Mitotic kinesins are attractive targets for new anti-cancer agents. Mitotic kinesins are enzymes essential for assembly and function of the mitotic spindle, but are not generally part of other microtubule structures, such as in nerve processes.

While a number of compounds have been described for use in treating cellular proliferative disease, there is an ongoing need to develop methods and compositions for treating cellular proliferative disease.

The present invention provides a method of treating cellular proliferative disease, such as cancer, hyperplasias, restenosis, cardiac hypertrophy, immune disorders and inflammation, comprising the administration of a chromenone derivative which is a mitotic kinesin (particularly KSP) modulator to a mammal in need thereof. More particularly, the present invention provides a method of treating cellular proliferative disease, such as above, comprising the administration of a chromenone derivative which is a mitotic kinesin (particularly KSP) inhibitor.

More particularly, the present invention relates to a method of treating cellular proliferative disease, comprising administering to a mammal in need thereof such a chromenone derivative, in combination with one or more chemotherapeutic agents selected from neutropenia treatment agents, alkylating agents, antimetabolites, platinating agents, topoisomerase inhibitors, tubulin agents, signalling inhibitors, and other chemotherapeutic agents.

The present invention also relates to pharmaceutical compositions, comprising such a chromenone derivative, one or more chemotherapeutic agents selected from neutropenia treatment agents, alkylating agents, antimetabolites, platinating agents, topoisomerase inhibitors, tubulin agents, signalling inhibitors, and other chemotherapeutic agents; and optionally one or more pharmaceutically acceptable excipients.

The methods and compositions of the invention may provide certain benefits, For example, the methods and compositions of the invention may exhibit improved aqueous solubility, chemical stability, drug absorption, therapeutic efficacy, clinical efficacy, toxicity profile, shelf life, manufacturability and/or formulation. For example, the methods and compositions of the invention may exhibit one or more of: greater aqueous solubility, chemical stability, sustained or prolonged drug or absorption levels, clinical efficacy, predictable toxicity, acceptable levels of dose-limiting toxicity, better shelf-life, better reproducibility in manufacturing and formulation, better therapeutic efficacy, etc.

The present invention relates to chromenone derivatives which are modulators (e.g., inhibitors) of a mitotic kinesin, particularly the mitotic kinesin KSP. In particular, the present invention relates to the use of such derivatives in the treatment of cellular proliferative diseases, such as cancer, hyperplasias, restenosis, cardiac hypertrophy, immune disorders and inflammation.

The present invention particularly relates to a method of treating cellular proliferative diseases, comprising administering to a mammal in need thereof such a chromenone derivative, in combination with a chemotherapeutic agent selected from neutropenia treatment agents, alkylating agents, antimetabolites, platinating agents, topoisomerase inhibitors, tubulin agents, signalling inhibitors, and other chemotherapeutic agents.

The present invention also relates to pharmaceutical compositions, comprising such a chromenone derivative, a chemotherapeutic agent selected from neutropenia treatment agents, alkylating agents, antimetabolites, platinating agents, topoisomerase inhibitors, tubulin agents, signalling inhibitors, and other chemotherapeutic agents; and optionally a pharmaceutically acceptable excipient.

The chromenone derivatives and other chemotherapeutic agents may also be administered in combination with other treatments, e.g., radiation.

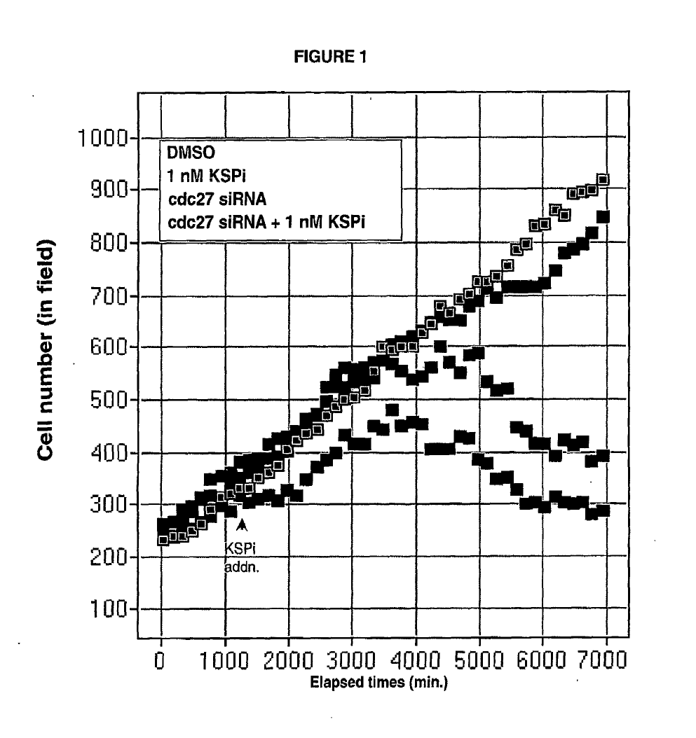

FIG. 1 shows graphical results of an extended timelapse analysis (5 days) of GFP-H2B SKOV3 cells transfected with cdc27 siRNA (t=0) and treated with concentration of KSP inhibitor below single agent effective dose (1 nM Compound B, @ t-18 h).

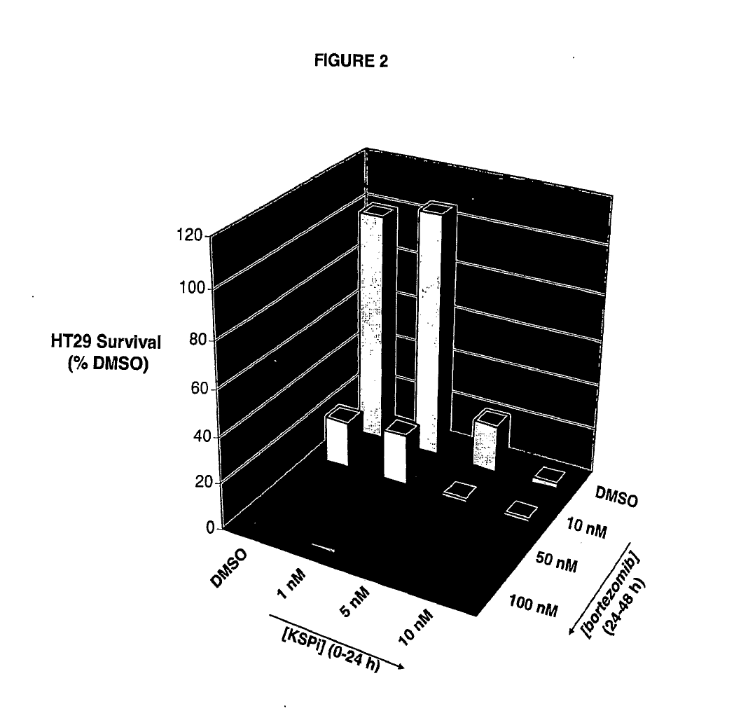

FIG. 2 shows graphical results of the comparison of 5 nM compound B and 10 nM bortezomib as single agents or in combination.

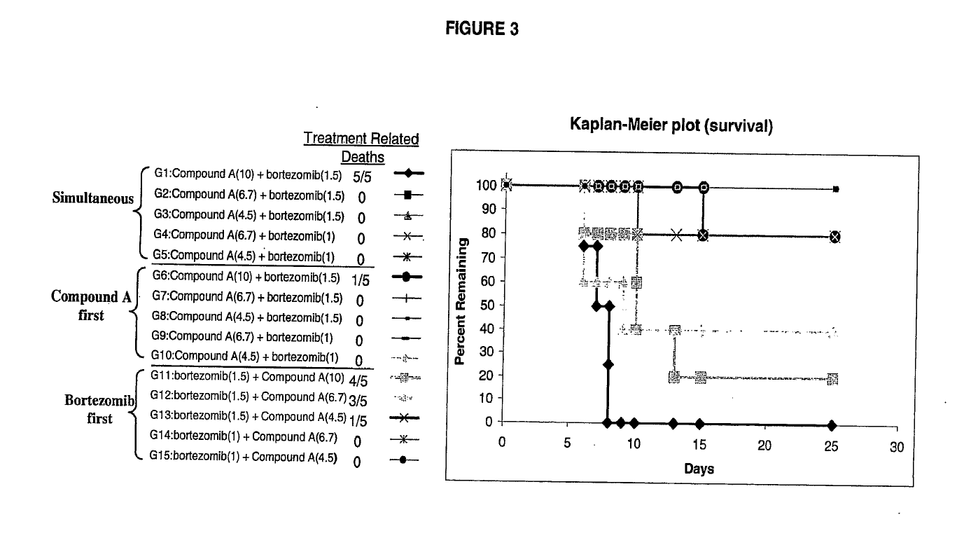

FIG. 3 shows graphical results of the in vivo tolerability of 4.5 mg/kg Compound A and 1.5 mg/kg bortezomib as single agents or in combination.

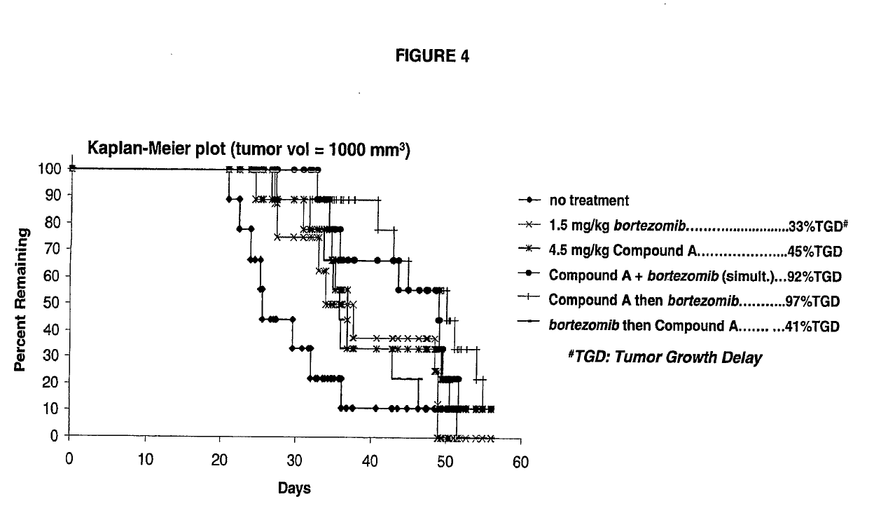

FIG. 4 shows graphical results of the comparison of 4.5 mg/kg Compound A and 1.5 mg/kg bortezomib as single agents or in combination.

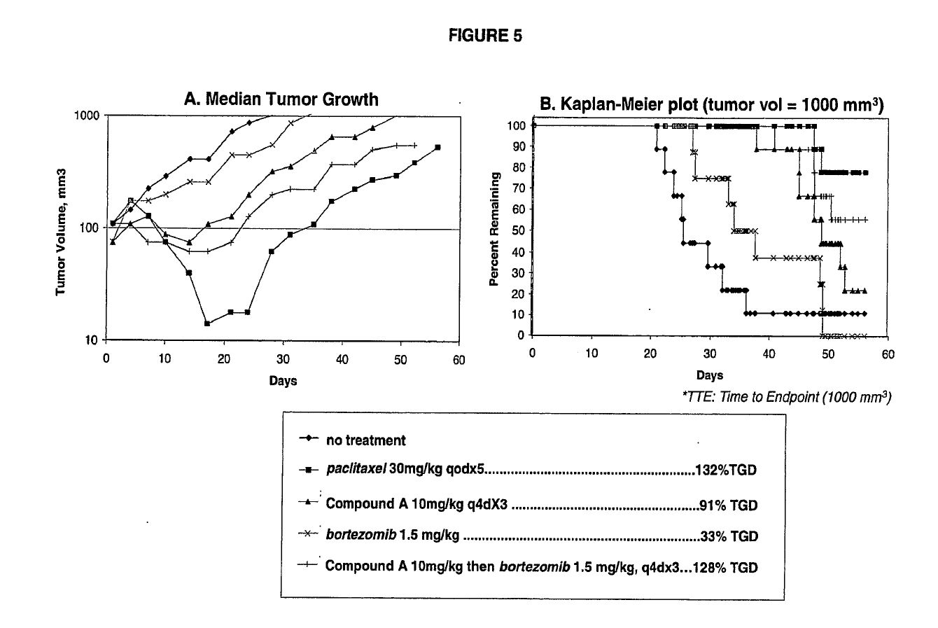

FIG. 5A shows graphical results of the comparison of 10 mg/kg Compound A and 1.5 mg/kg bortezomib as single agents

FIG. 5B shows graphical results of the comparison of 10 mg/kg Compound B and 1.5 mg/kg bortezomib in combination.

In one embodiment, the chromenone derivatives useful in the present invention are selected from compounds represented by Formula (I):

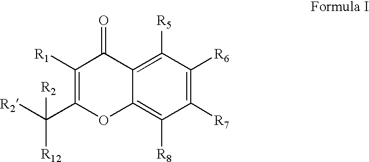

wherein:

R₁ is chosen from hydrogen, optionally substituted alkyl-, optionally substituted aryl-, optionally substituted aralkyl-, optionally substituted heteroaryl-, and optionally substituted heteroaralkyl-;

R₂ and R_{2′} are independently chosen from hydrogen, optionally substituted alkyl-, optionally substituted alkoxy, optionally substituted aryl-, optionally substituted aralkyl-, optionally substituted heteroaryl-, and optionally substituted heteroaralkyl-; or R₂ and R_{2′} taken together form an optionally substituted 3- to 7-membered ring;

R₁₂ is selected from the group consisting of optionally substituted imidazolyl, optionally substituted imidazolinyl, —NHR₄; —N(R₄)(COR₃); —N(R₄)(SO₂R₃ₐ); and —N(R₄)(CH₂R_{3b});

R₃ is chosen from hydrogen, optionally substituted alkyl-, optionally substituted aryl-, optionally substituted aralkyl-, optionally substituted heteroaryl-, optionally substituted heteroaralkyl-, R₁₅O— and R₁₇—NH—;

R₃ₐ is chosen from optionally substituted alkyl-, optionally substituted aryl-, optionally substituted aralkyl-, optionally substituted heteroaryl-, optionally substituted heteroaralkyl-, and R₁₇—NH—;

R_{3b} is chosen from hydrogen, optionally substituted alkyl-, optionally substituted aryl-, optionally substituted aralkyl-, optionally substituted heteroaryl-, and optionally substituted heteroaralkyl-;

R₄ is chosen from hydrogen, optionally substituted alkyl-, optionally substituted aryl-, optionally substituted aralkyl-, optionally substituted heterocyclyl-, and optionally substituted heteroaralkyl-;

R₅, R₆, R₇ and R₈ are independently chosen from hydrogen, acyl, optionally substituted alkyl-, optionally substituted alkoxy, halogen, hydroxyl, nitro, cyano, dialkylamino, alkylsulfonyl-, alkylsulfonamido-, alkylthio-, carboxyalkyl-, carboxamido-, aminocarbonyl-, optionally substituted aryl and optionally substituted heteroaryl-;

R₁₅ is chosen from optionally substituted alkyl-, optionally substituted aryl-, optionally substituted aralkyl-, optionally substituted heteroaryl-, and optionally substituted heteroaralkyl-; and

R₁₇ is hydrogen, optionally substituted alkyl-, optionally substituted aryl-, optionally substituted aralkyl-, optionally substituted heteroaryl-, or optionally substituted hetero-aralkyl.

Compounds of Formula (I) and pharmaceutically acceptable salts thereof are described, for example, in U.S. Pat. No. 6,924,376, incorporated herein by reference in its entirety.

Alkyl is intended to include linear, branched, or cyclic aliphatic hydrocarbon structures and combinations thereof, which structures may be saturated or unsaturated. Lower-alkyl refers to alkyl groups of from 1 to 5 carbon atoms, such as from 1 to 4 carbon atoms. Examples of lower-alkyl groups include methyl, ethyl, propyl, isopropyl, butyl, s- and t-butyl and the like. In some embodiments, alkyl groups are those of C₁₃ or below. Cycloalkyl is a subset of alkyl and includes cyclic aliphatic hydrocarbon groups of from 3 to 13 carbon atoms. Examples of cycloalkyl groups include c-propyl, c-butyl, c-pentyl, norbornyl, adamantyl and the like. Cycloalkyl-alkyl- is another subset of alkyl and refers to cycloalkyl attached to the parent structure through a non-cyclic alkyl. Examples of cycloalkyl-alkyl- include cyclohexylmethyl, cyclopropylmethyl, cyclohexylpropyl, and the like. In this application, alkyl includes alkanyl, alkenyl and alkynyl residues; it is intended to include vinyl, allyl, isoprenyl and the like. Alkylene-, alkenylene, and alkynylene- are other subsets of alkyl, including the same residues as alkyl, but having two points of attachment within a chemical structure. Examples of alkylene include ethylene (—CH₂CH₂—), propylene (—CH₂CH₂CH₂—), dimethylpropylene (—CH₂C(CH₃)₂CH₂—) and cyclohexylpropylene (—CH₂CH₂CH(C₆H₁₃)—). Likewise, examples of alkenylene include ethenylene (—CH═CH—), propenylene (—CH═CH—CH₂—), and cyclohexylpropenylene (—CH═CHCH(C₆H₁₃)—). Examples of alkynylene include ethynylene (—C≡C—) and propynylene (—CH≡CH—CH₂—). When an alkyl residue having a specific number of carbons is named, all geometric isomers having that number of carbons are intended to be encompassed; thus, for example, “butyl” is meant to include n-butyl, sec-butyl, isobutyl and t-butyl; “propyl” includes n-propyl, isopropyl, and c-propyl.

Alkoxy or alkoxyl refers to an alkyl group, such as those groups including from 1 to 8 carbon atoms, of a straight, branched, or cyclic configuration, or a combination thereof, attached to the parent structure through an oxygen (i.e., the group alkyl-O—). Examples include methoxy-, ethoxy-, propoxy-, isopropoxy-, cyclopropyloxy-, cyclohexyloxy- and the like. Lower-alkoxy refers to alkoxy groups containing one to four carbons.

Acyl refers to groups of from 1 to 8 carbon atoms of a straight, branched, or cyclic configuration or a combination thereof, attached to the parent structure through a carbonyl functionality. Such groups may be saturated or unsaturated, and aliphatic or aromatic. One or more carbons in the acyl residue may be replaced by nitrogen, oxygen or sulfur as long as the point of attachment to the parent remains at the carbonyl. Examples include acetyl, benzoyl, propionyl, isobutyryl, t-butoxycarbonyl, benzyloxycarbonyl and the like. Lower-acyl refers to acyl groups containing one to four carbons.

Amino refers to the group —NH₂. The term “substituted amino” refers to the group —NHR or —NRR where each R is independently selected from the group: optionally substituted alkyl, optionally substituted alkoxy, optionally substituted amino carbonyl, optionally substituted aryl, optionally substituted heteroaryl, optionally substituted heterocyclyl, acyl, alkoxycarbonyl, sulfanyl, sulfinyl and sulfonyl, e.g., diethylamino, methylsulfonylamino, furanyl-oxy-sulfonamino.

Aminocarbonyl- refers to the group —NR^{c}COR^{b}, NR^{c}CO₂R^{b}, or —NR^{c}CONR^{b}R^{c}, where

R^{b} is H or optionally substituted C₁-C₆ alkyl, aryl, heteroaryl, aryl-C₁-C₄ alkyl-, or heteroaryl-C₁-C₄ alkyl- group; and

R^{c} is hydrogen or C₁-C₄ alkyl; and where each optionally substituted R^{b} group is independently unsubstituted or substituted with one or more substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halogen, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ phenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

Aryl and heteroaryl mean a 5- or 6-membered aromatic or heteroaromatic ring containing 0 or 1-4 heteroatoms, respectively, selected from O, N, or S; a bicyclic 9- or 10-membered aromatic or heteroaromatic ring system containing 0 or 1-4 (or more) heteroatoms, respectively, selected from O, N, or S; or a tricyclic 12- to 14-membered aromatic or heteroaromatic ring system containing 0 or 1-4 (or more) heteroatoms, respectively, selected from O, N, or S. The aromatic 6- to 14-membered carbocyclic rings include, e.g., phenyl, naphthyl, indanyl, tetralinyl, and fluorenyl and the 5- to 10-membered aromatic heterocyclic rings include, e.g., imidazolyl, pyridinyl, indolyl, thienyl, benzopyranonyl, thiazolyl, furanyl, benzimidazolyl, quinolinyl, isoquinolinyl, quinoxalinyl, pyrimidinyl, pyrazinyl, tetrazolyl and pyrazolyl.

Aralkyl- refers to a residue in which an aryl moiety is attached to the parent structure via an alkyl residue. Examples include benzyl, phenethyl, phenylvinyl, phenylallyl and the like. Heteroaralkyl- refers to a residue in which a heteroaryl moiety is attached to the parent structure via an alkyl residue. Examples include furanylmethyl, pyridinylmethyl, pyrimidinylethyl and the like.

Aralkoxy- refers to the group —O-aralkyl. Similarly, heteroaralkoxy-refers to the group —O-heteroaralkyl; aryloxy- refers to the group —O-aryl; acyloxy-refers to the group —O-acyl; heteroaryloxy- refers to the group —O-heteroaryl; and heterocyclyloxy- refers to the group —O-heterocyclyl (i.e., aralkyl, heteroaralkyl, aryl, acyl, heterocyclyl, or heteroaryl is attached to the parent structure through an oxygen).

Carboxyalkyl- refers to the group -alkyl-COOH.

Carboxamido refers to the group —CONR^{b}R^{c}, where

R^{b} is H or optionally substituted C₁-C₆ alkyl, aryl, heteroaryl, aryl-C₁-C₄ alkyl-, or heteroaryl-C₁-C₄ alkyl- group; and

R^{c} is hydrogen or C₁-C₄ alkyl; and

where each optionally substituted R^{b} group is independently unsubstituted or substituted with one or more substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halogen, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ phenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

Halogen or halo refers to fluorine, chlorine, bromine or iodine. Fluorine, chlorine and bromine are preferred. Dihaloaryl, dihaloalkyl, trihaloaryl etc. refer to aryl and alkyl substituted with the designated plurality of halogens (here, 2, 2 and 3, respectively), but not necessarily a plurality of the same halogen; thus 4-chloro-3-fluorophenyl is within the scope of dihaloaryl.

Heterocyclyl means a cycloalkyl or aryl residue in which one to four of the carbons is replaced by a heteroatom such as oxygen, nitrogen or sulfur. Examples of heterocycles that fall within the scope of the invention include azetidinyl, imidazolinyl, pyrrolidinyl, pyrazolyl, pyrrolyl, indolyl, quinolinyl, isoquinolinyl, tetrahydroisoquinolinyl, benzofuranyl, benzodioxanyl, benzodioxyl (commonly referred to as methylenedioxyphenyl, when occurring as a substituent), tetrazolyl, morpholinyl, thiazolyl, pyridinyl, pyridazinyl, piperidinyl, pyrimidinyl, thienyl, furanyl, oxazolyl, oxazolinyl, isoxazolyl, dioxanyl, tetrahydrofuranyl and the like. “N-heterocyclyl” refers to a nitrogen-containing heterocycle. The term heterocyclyl encompasses heteroaryl, which is a subset of heterocyclyl. Examples of N-heterocyclyl residues include azetidinyl, 4-morpholinyl, 4-thiomorpholinyl, 1-piperidinyl, 1-pyrrolidinyl, 3-thiazolidinyl, piperazinyl and 4-(3,4-dihydrobenzoxazinyl). Examples of substituted heterocyclyl include 4-methyl-1-piperazinyl and 4-benzyl-1-piperidinyl.

Optional or optionally means that the subsequently described event or circumstance may or may not occur, and that the description includes instances where said event or circumstances occurs and instances in which it does not. For example, “optionally substituted alkyl” includes “alkyl” and “substituted alkyl” as defined herein. It will be understood by those skilled in the art with respect to any group containing one or more substituents that such groups are not intended to introduce any substitution or substitution patterns that are sterically impractical and/or synthetically non-feasible and/or inherently unstable.

Substituted alkoxy refers to alkoxy wherein the alkyl constituent is substituted (i.e., —O-(substituted alkyl)). One substituted alkoxy group is “polyalkoxy” or —O-(optionally substituted alkylene)-(optionally substituted alkoxy), and includes groups such as —OCH₂CH₂OCH₃, and residues of glycol ethers such as polyethyleneglycol, and —O(CH₂CH₂O)ₓCH₃, where x is an integer of about 2-20, such as about 2-10, for example, about 2-5. Another substituted alkoxy group is hydroxyalkoxy or —OCH₂(CH₂)_{y}OH, where y is an integer of about 1-10, such as about 1-4.

Substituted- alkyl, aryl, and heteroaryl, which includes the substituted alkyl, aryl and heteroaryl moieties of any group containing an optionally substituted alkyl, aryl and heteroaryl moiety (e.g., alkoxy, aralkyl and heteroaralkyl), refer respectively to alkyl, aryl, and heteroaryl wherein one or more (up to about 5, such as up to about 3) hydrogen atoms are replaced by a substituent independently selected from the group:

—R^{a}, —OR^{b}, —O(C₁-C₂ alkyl)O— (as an aryl substituent), —SR^{b}, —NR^{b}R^{c}, halogen, cyano, nitro, —COR^{b}, —CO₂R^{b}, —CONR^{b}R^{c}, —OCOR^{b}, —OCO₂R^{b}, —OCONR^{b}R^{c}, —NR^{c}COR^{b}, —NR^{c}CO₂R^{b}, —NR^{c}CONR^{b}R^{c}, —CO₂R^{b}, —CONR^{b}R^{c}, —NR^{c}COR^{b}, —SOR^{a}, —SO₂R^{a}, —SO₂NR^{b}R^{c}, and —NR^{c}SO₂R^{a},

where R^{a} is an optionally substituted C₁-C₆ alkyl, aryl, heteroaryl, aryl-C₁-C₄ alkyl-, or heteroaryl-C₁-C₄ alkyl- group,

R^{b} is H or optionally substituted C₁-C₆ alkyl, aryl, heteroaryl, aryl-C₁-C₄ alkyl-, or heteroaryl-C₁-C₄ alkyl- group;

R^{c} is hydrogen or C₁-C₄ alkyl;

where each optionally substituted R^{a} group and R^{b} group is independently unsubstituted or substituted with one or more substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halogen, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ phenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

Sulfanyl refers to the groups: —S-(optionally substituted alkyl), —S-(optionally substituted aryl), —S-(optionally substituted heteroaryl), and —S-(optionally substituted heterocyclyl).

Sulfinyl refers to the groups: —S(O)—H, —S(O)-(optionally substituted alkyl), —S(O)-optionally substituted aryl), —S(O)-(optionally substituted heteroaryl), —S(O)-(optionally substituted heterocyclyl); and —S(O)-(optionally substituted amino).

Sulfonyl refers to the groups: —S(O₂)—H, —S(O₂)-(optionally substituted alkyl), —S(O₂)-optionally substituted aryl), —S(O₂)-(optionally substituted heteroaryl), —S(O₂)-(optionally substituted heterocyclyl), —S(O₂)-(optionally substituted alkoxy), —S(O₂)-optionally substituted aryloxy), —S(O₂)-(optionally substituted heteroaryloxy), —S(O₂)-(optionally substituted heterocyclyloxy); and —S(O₂)-(optionally substituted amino).

Pharmaceutically acceptable salts of the compounds in accordance with the present invention, such as encompassed by Formula (I), may include those derived from pharmaceutically acceptable inorganic and organic acids or from other base addition salts. For example, a suitable pharmaceutically acceptable salt of compounds of formula (I) is the hydrochloride salt(s).

Other acids, while not in themselves pharmaceutically acceptable, may be useful as intermediates in obtaining the compounds of the invention and their pharmaceutically acceptable acid addition salts.

Suitable inorganic acids may include the following acids: hydrochloric, hydrobromic, sulfuric, and phosphoric acids. Suitable organic acids may include the following acids: acetic, propionic, glycolic, lactic, pyruvic, malonic, succinic, fumaric, malic, tartaric, citric, cyclamic, ascorbic, maleic, hydroxymaleic, dihydroxymaleic, benzoic, phenylacetic, 4-aminobenzoic, anthranilic, cinnamic, salicylic, 4-aminosalicylic, 2-phenoxybenzoic, 2-acetoxybenzoic, mandelic, sulfonic, methanesulfonic, ethanesulfonic, P-hydroxyethane-sulfonic acids and the like.

Non-toxic salts of compounds of the present invention formed with inorganic and organic bases may include salts of alkali metals (such as sodium, potassium, lithium, etc.), alkaline earth metals (such as calcium, magnesium, etc.), light metals of group IIIA (such as aluminum, etc.), organic amines (such as primary, secondary, or tertiary amine salts, etc.) and the like.

Chromenones useful in the present invention may contain one or more asymmetric centers (e.g., in one embodiment of Formula I the carbon to which R₂ and R_{2′} are attached), which may give rise to enantiomers, diastereomers, and other stereoisomeric forms that may be defined, in terms of absolute stereochemistry, as (R)- or (S)-. The present invention is meant to include all such possible isomers, including racemic mixtures, optically pure forms and intermediate mixtures.

In one embodiment of Formula I, R₂ and R_{2′} are each attached to a stereogenic center having an R-configuration.

When considering the compounds of Formula I, in some embodiments, when either one or both R₂ or R_{2′} is not hydrogen (in some embodiments, either one of R₂ or R_{2′} is not hydrogen), R₁ is selected from hydrogen, optionally substituted C₁-C₈ alkyl, optionally substituted aryl, optionally substituted heteroaryl, optionally substituted aryl-C₁-C₄-alkyl-, and optionally substituted heteroaryl-C₁-C₄-alkyl- (some embodiments, optionally substituted aryl and optionally substituted aryl-C₁-C₄-alkyl-). In some embodiments, R₁ is selected from hydrogen, optionally substituted C₁-C₄ alkyl, optionally substituted phenyl-C₁-C₄-alkyl-, optionally substituted naphthalenylmethyl, optionally substituted phenyl, and naphthyl. In some embodiments, R₁ is optionally substituted phenyl-C₁-C₄-alkyl- or optionally substituted heteroaryl-C₁-C₄-alkyl-.

In some embodiments, R₁ is naphthyl, phenyl, bromophenyl, chlorophenyl, methoxyphenyl, ethoxyphenyl, tolyl, dimethylphenyl, chorofluorophenyl, methylchlorophenyl, ethylphenyl, phenethyl, benzyl, chlorobenzyl, methylbenzyl, methoxybenzyl, cyanobenzyl, hydroxybenzyl, dichlorobenzyl, dimethoxybenzyl, or naphthalenylmethyl. In some embodiments, R₁ is benzyl, cyanobenzyl, methoxybenzyl, or naphthalenylmethyl. In some embodiments, R₁ is benzyl.

In some embodiments wherein R₂ and R_{2′} are both hydrogen, R₁ is chosen from optionally substituted aryl, optionally substituted aryl-C₁-C₄-alkyl-, optionally substituted heteroaryl, and optionally substituted heteroaryl-C₁-C₄-alkyl-, provided however, that R₁ is not substituted phenyl. In some embodiments, R₁ is optionally substituted aryl-C₁-C₄-alkyl- or optionally substituted heteroaryl-C₁-C₄-alkyl-. In some embodiments, when R₂ and R_{2′} are both hydrogen, R₁ is selected from optionally substituted phenyl-C₁-C₄-alkyl, and optionally substituted naphthalenylmethyl. In some embodiments, wherein R₂ and R_{2′} are both hydrogen, R₁ is chosen from benzyl, chlorobenzyl, methylbenzyl, methoxybenzyl, cyanobenzyl, hydroxybenzyl, dichlorobenzyl, dimethoxybenzyl, and naphthalenylmethyl. In some embodiments, R₁ is benzyl, cyanobenzyl, methoxybenzyl, or naphthalenylmethyl. In some embodiments, R₁ is benzyl.

When considering the compounds of Formula I and as will be appreciated by those skilled in the art, the compounds described herein possess a potentially chiral center at the carbon to which R₂ and R_{2′} are attached. The R₂ and R_{2′} groups may be the same or different; if different, the compound is chiral (i.e., has a stereogenic center). When R₂ and R_{2′} are different, in some embodiments, R₂ is hydrogen and R_{2′} is other than hydrogen. The invention contemplates the use of pure enantiomers and mixtures of enantiomers, including racemic mixtures, although the use of a substantially optically pure enantiomer will generally be preferred. The term “substantially optically pure” or “enantiomerically pure” means having at least about 95% of the described enantiomer with no single impurity greater than about 1% and in some embodiments, at least about 97.5% enantiomeric excess. In some embodiments, the stereogenic center to which R₂ and R_{2′} are attached is of the R configuration.

In one embodiment, R₂ is optionally substituted C₁-C₄ alkyl, and R_{2′} is hydrogen or optionally substituted C₁-C₄ alkyl. In some embodiments, R_{2′} is hydrogen and R₂ is optionally substituted C₁-C₄ alkyl. In some embodiments, R₂ is chosen from methyl, ethyl, propyl (such as c-propyl or i-propyl), butyl (such as t-butyl), methylthioethyl, methylthiomethyl, aminobutyl, (CBZ)aminobutyl, cyclohexylmethyl, benzyloxymethyl, methylsulfinylethyl, methylsulfinylmethyl, and hydroxymethyl, and R_{2′} is hydrogen. In some embodiments, R_{2′} is hydrogen and R₂ is ethyl or propyl (such as c-propyl or i-propyl). In some embodiments, R₂ is i-propyl. In some embodiments, the stereogenic center to which R₂ and R_{2′} is attached is of the R configuration.

In another embodiment, both R₂ and R_{2′} are hydrogen.

In some embodiments, R₃ is selected from optionally substituted C₁-C₈ alkyl, optionally substituted aryl-C₁-C₄-alkyl-, optionally substituted heteroaryl-C₁-C₄-alkyl-, optionally substituted heteroaryl, optionally substituted aryl, R₁₅O— and R₁₇—NH—, R₁₅ is chosen from optionally substituted C₁-C₈ alkyl and optionally substituted aryl, and R₁₇ is chosen from hydrogen, optionally substituted C₁-C₈ alkyl and optionally substituted aryl. In some embodiments, R₃ is chosen from optionally substituted C₁-C₈ alkyl (e.g., C₁-C₈ alkyl substituted with lower-alkoxy), optionally substituted heteroaryl, and optionally substituted aryl.

In some embodiments, when R₃ is not R₁₇NH— or R₁₅O—, R₃ is chosen from phenyl; phenyl substituted with one or more of the following substituents: halo, C₁-C₄ alkyl, C₁-C₄ alkyl substituted with hydroxy (e.g., hydroxymethyl), C₁-C₄ alkoxy, C₁-C₄ alkyl substituted with C₁-C₄ alkoxy, nitro, formyl, carboxy, cyano, methylenedioxy, ethylenedioxy, acyl (e.g., acetyl), —N-acyl (e.g., N-acetyl) or trifluoromethyl; benzyl; phenoxymethyl-; halophenoxymethyl-; phenylvinyl-; heteroaryl-; heteroaryl- substituted with C₁-C₄ alkyl or C₁-C₄ alkyl substituted with halo (e.g., CF₃); C₁-C₄ alkyl substituted with C₁-C₄ alkoxy- and benzyloxymethyl-.

In some embodiments, when R₃ is not R₁₇NH— or R₁₅O—, R₃ is chosen from phenyl, halophenyl, dihalophenyl, cyanophenyl, halo(trifluoromethyl)phenyl, hydroxymethylphenyl, methoxyphenyl, ethoxyphenyl, carboxyphenyl, ethylphenyl, tolyl, methylenedioxyphenyl, ethienedixoyphenyl, methoxychlorophenyl, dihydro-benzodioxinyl, methylhalophenyl, trifluoromethylphenyl, bis(trifluoromethyl)phenylbenzyl, furanyl, C₁-C₄ alkyl substituted furanyl, trifluoromethylfuranyl, C₁-C₄ alkyl substituted trifluoromethylfuranyl, benzofuranyl, thiophenyl, C₁-C₄ alkyl substituted thiophenyl, benzothiophenyl, benzothiadiazolyl, pyridinyl, indolyl, methylpyridinyl, trifluoromethylpyridinyl, pyrrolyl, quinolinyl, picolinyl, pyrazolyl, C₁-C₄ alkyl substituted pyrazolyl, N-methylpyrazolyl, C₁-C₄ alkyl substituted N-methylpyrazolyl, C₁-C₄ alkyl substituted pyrazinyl, C₁-C₄ alkyl substituted isoxazolyl, benzoisoxazolyl, morpholinomethyl, methylthiomethyl, methoxymethyl, N-methyl imidazolyl, and imidazolyl. In some embodiments, R₃ is tolyl, halophenyl, halomethylphenyl, hydroxymethylphenyl, methylenedioxyphenyl, formylphenyl or cyanophenyl.

In some embodiments, when R₃ is R₁₇NH—, R₁₇ is chosen from hydrogen, C₁-C₄ alkyl; cyclohexyl; phenyl; and phenyl substituted with halo, C₁-C₄ alkyl, C₁-C₄ alkoxy, or C₁-C₄ alkylthio.

In some embodiments, when R₃ is R₁₇NH—, R₁₇ is hydrogen isopropyl, butyl, cyclohexyl, phenyl, bromophenyl, dichlorophenyl, methoxyphenyl, ethylphenyl, tolyl, trifluoromethylphenyl or methylthiophenyl.

In some embodiments, wherein R₃ is R₁₅O—, R₁₅ is chosen from optionally substituted C₁-C₈ alkyl and optionally substituted aryl.

In some embodiments, when R₁₂ is —N(R₄)(SO₂R₃ₐ), R₃ₐ is chosen from C₁-C₁₃ alkyl; phenyl; naphthyl; phenyl substituted with halo, C₁-C₄ alkyl, C₁-C₄ alkoxy, cyano, nitro, methylenedioxy, or trifluoromethyl; biphenylyl and heteroaryl. In some embodiments, R₃ₐ is chosen from phenyl substituted with halo, C₁-C₄ alkyl, C₁-C₄ alkoxy, cyano, nitro, methylenedioxy, or trifluoromethyl and naphthyl.

In some embodiments, when R₁₂ is —N(R₄)(CH₂R_{3b}), R_{3b} is chosen from C₁-C₁₃ alkyl; substituted C₁-C₄ alkyl; phenyl; naphthyl; phenyl substituted with carboxy, alkoxycarbonyl cyano, halo, C₁-C₄ alkyl-, C₁-C₄ alkoxy, nitro, methylenedioxy, or trifluoromethyl; biphenylyl, benzyl; and heterocyclyl.

In some embodiments, R_{3b} is chosen from halophenyl, polyhalophenyl, methylhalophenyl, tolyl, dimethylphenyl, methoxyphenyl, dimethoxyphenyl, cyanophenyl, trifluoromethylphenyl, trifluoromethoxyphenyl, bis(trifluoromethyl)phenyl, carboxyphenyl, t-butylphenyl, methoxycarbonylphenyl, piperidinyl and naphthyl.

In some embodiments, when R₁₂ is —NHR₄, —N(R₄)(COR₃), or —N(R₄)(CH₂R_{3b}), R₄ is chosen from hydrogen, optionally substituted C₁-C₁₃ alkyl, optionally substituted aryl, optionally substituted aryl-C₁-C₄-alkyl-, optionally substituted heterocyclyl, and optionally substituted heteroaryl-C₁-C₄-alkyl- (in some embodiments, hydrogen or optionally substituted C₁-C₁₃ alkyl).

In some embodiments, R₄ is chosen from hydrogen, C₁-C₄ alkyl; cyclohexyl; phenyl substituted with hydroxyl, C₁-C₄ alkoxy or C₁-C₄ alkyl; benzyl; heteroarylmethyl-; heteroarylethyl-; heteroarylpropyl-; and R₁₆-alkylene-, wherein R₁₆ is hydroxyl, di(C₁-C₄ alkyl)amino-, (C₁-C₄ alkyl)amino-, amino, C₁-C₄ alkoxy-, or N-heterocyclyl-, such aspyrrolidino, piperidino or imidazolyl.

In some embodiments, R₄ is R₁₆-alkylene-, wherein R₁₆ is amino, C₁-C₄ alkylamino-, di(C₁-C₄ alkyl)amino-, C₁-C₄ alkoxy-, hydroxyl, or N-heterocyclyl. In some embodiments, R₁₆ is amino.

In some embodiments, when R₁₂ is —NHR₄, —N(R₄)(COR₃), or —N(R₄)(CH₂R_{3b}), R₄ is chosen from hydrogen, methyl, ethyl, propyl, butyl, cyclohexyl, carboxyethyl, carboxymethyl, methoxyethyl, hydroxyethyl, hydroxypropyl, dimethylaminoethyl, dimethylaminopropyl, diethylaminoethyl, diethylaminopropyl, aminopropyl, methylaminopropyl, 2,2-dimethyl-3-(dimethylamino)propyl, 1-cyclohexyl-4-(diethylamino)butyl, aminoethyl, aminobutyl, aminopentyl, aminohexyl, aminoethoxyethyl, isopropylaminopropyl, diisopropylaminoethyl, 1-methyl-4-(diethylamino)butyl, (t-Boc)aminopropyl, hydroxyphenyl, benzyl, methoxyphenyl, methylmethoxyphenyl, dimethylphenyl, tolyl, ethylphenyl, (oxopyrrolidinyl)propyl, (methoxycarbonyl)ethyl, benzylpiperidinyl, pyridinylethyl, pyridinylmethyl, morpholinylethyl morpholinylpropyl, piperidinyl, azetidinylmethyl, azetidinylethyl, azetidinylpropyl pyrrolidinylethyl, pyrrolidinylpropyl, piperidinylmethyl, piperidinylethyl, imidazolylpropyl, imidazolylethyl, (ethylpyrrolidinyl)methyl, (methylpyrrolidinyl)ethyl, (methylpiperidinyl)propyl, (methylpiperazinyl)propyl, furanylmethyl and indolylethyl.

In some embodiments, R₄ is aminoethyl, aminopropyl, aminobutyl, aminopentyl, aminohexyl, methylaminoethyl, methylaminopropyl, methylaminobutyl, methylaminopentyl, methylaminohexyl, dimethylaminoethyl, dimethylaminopropyl, dimethylaminobutyl, dimethylaminopentyl, dimethylaminohexyl, ethylaminoethyl, ethylaminopropyl, ethylaminobutyl, ethylaminopentyl, ethylaminohexyl, diethylaminoethyl, diethylaminopropyl, diethylaminobutyl, diethylaminopentyl, or diethylaminohexyl, and in some embodiments, aminopropyl.

In some embodiments, when R₁₂ is —N(R₄)(SO₂R₃ₐ), R₄ is chosen from C₁-C₄ alkyl, cyclohexyl; phenyl substituted with hydroxyl, C₁-C₄ alkoxy or C₁-C₄ alkyl; benzyl; heteroarylmethyl-; heteroarylethyl-; heteroarylpropyl-; heteroarylethyl-; heteroarylpropyl- and R₁₆-alkylene-, wherein R₁₆ is hydroxyl, di(C₁-C₄ alkyl)amino-, (C₁-C₄ alkyl)amino-, amino, C₁-C₄ alkoxy-, or N-heterocyclyl-, such as pyrrolidino, piperidino or imidazolyl.

In some embodiments, when R₁₂ is an imidazole, R₁₂ has the formula:

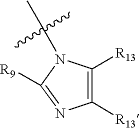

wherein

R₉ is chosen from hydrogen, optionally substituted C₁-C₈ alkyl, optionally substituted aryl, optionally substituted aryl-C₁-C₄-alkyl-, optionally substituted heteroaryl-C₁-C₄-alkyl-, optionally substituted aryl-C₁-C₄-alkoxy-, optionally substituted heteroaryl-C₁-C₄-alkoxy-, optionally substituted heteroaryl-; and R₁₃ and R_{13′} are independently hydrogen, optionally substituted C₁-C₈ alkyl, optionally substituted aryl, or optionally substituted aryl-C₁-C₄-alkyl- (in some embodiments, optionally substituted aryl). In some embodiments, R₉ is phenyl substituted with C₁-C₄-alkyl, C₁-C₄-alkoxy-, and/or halo (such as C₁-C₄-alkyl and/or halo); phenyl; or benzyl. In some embodiments, R₉ is tolyl; halophenyl; or halomethylphenyl.

In some embodiments, R₁₃ is hydrogen and R_{13′} is substituted C₁-C₄ alkyl. In some embodiments, R₁₃ is hydrogen and R_{13′} is aminomethyl, aminoethyl, aminopropyl, acetylamino-methyl, acetylaminoethyl, benzyloxycarbonylamino-methyl or benzyloxycarbonylamino-ethyl.

In some embodiments, when R₁₂ is an imidazoline, R₁₂ has the formula

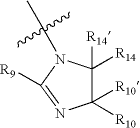

wherein R₉ is chosen from hydrogen, optionally substituted C₁-C₈ alkyl, optionally substituted aryl, optionally substituted aryl-C₁-C₄-alkyl-, and optionally substituted heteroaryl-; and R₁₀, R_{10′}, R₁₄, and R_{14′} are independently chosen from hydrogen, optionally substituted C₁-C₈ alkyl, optionally substituted aryl, and optionally substituted aryl-C₁-C₄-alkyl-. In some embodiments, R₉ is methylenedioxyphenyl; phenyl; phenyl substituted with C₁-C₄ alkyl, C₁-C₄ alkoxy, and/or halo; or benzyl. In some embodiments, R₉ is methylenedioxyphenyl-; phenyl; or phenyl substituted with methoxy, halo and/or methyl (in some embodiments, halo and/or methyl, including tolyl), and in some embodiments, methylenedioxyphenyl or said substituted phenyls. In some embodiments, R₁₀, R_{10∝0}, R_{14′}, and R₁₄ are independently hydrogen or optionally substituted alkyl (in some embodiments, optionally substituted C₁-C₄ alkyl). In some embodiments, R₁₀ and R_{10′} are independently selected from the group consisting of hydrogen or optionally substituted C₁-C₄ alkyl (and such as methyl or aminoalkyl-) and R_{14′} and R₁₄ are hydrogen.

In some embodiments, R₅, R₆, R₇ and R₈ are independently chosen from hydrogen; acyl, alkyl; alkyl substituted with alkyl, alkoxy, halo, hydroxyl, nitro, cyano, dialkylamino, alkylsulfonyl, alkylsulfonamido, alkylthio, carboxyalkyl, carboxamido, aminocarbonyl, lower-alkylaminocarbonyl- (e.g. methylaminocarbonyl- or ethylaminocarbonyl-), di(lower-alkyl)aminocarbonyl- (e.g. dimethylaminocarbonyl- or diethylaminocarbonyl-), aryl, or heteroaryl; alkoxy; alkoxy substituted with alkyl, acyl, alkoxy, halo, hydroxyl, nitro, cyano, dialkylamino, alkylsulfonyl, alkylsulfonamido, alkylthio, carboxyalkyl, carboxamido, aminocarbonyl, lower-alkylaminocarbonyl- (e.g. methylaminocarbonyl- or ethylaminocarbonyl-), di(lower-alkyl)aminocarbonyl- (e.g. dimethylaminocarbonyl- or diethylaminocarbonyl-), aryl, or heteroaryl; halogen; hydroxyl; nitro; cyano; dialkylamino; alkylsulfonyl; alkylsulfonamido; alkylthio; carboxyalkyl; carboxamido; amidocarbonyl; aryl; aryl substituted with alkyl, acyl, alkoxy, halo, hydroxyl, nitro, cyano, dialkylamino, alkylsulfonyl, alkylsulfonamido, alkylthio, carboxyalkyl, carboxamido, aminocarbonyl, lower-alkylaminocarbonyl- (e.g. methylaminocarbonyl- or ethylaminocarbonyl-), di(lower-alkyl)aminocarbonyl- (e.g. dimethylaminocarbonyl- or diethylaminocarbonyl-), aryl, or heteroaryl; heteroaryl or heteroaryl substituted with alkyl, acyl, alkoxy, halo, hydroxyl, nitro, cyano, dialkylamino, alkylsulfonyl, alkylsulfonamido, alkylthio, carboxyalkyl, carboxamido, aminocarbonyl, lower-alkylaminocarbonyl- (e.g. methylaminocarbonyl- or ethylaminocarbonyl-), di(lower-alkyl)aminocarbonyl- (e.g. dimethylaminocarbonyl- or diethylaminocarbonyl-), aryl, or heteroaryl.

In some embodiments, R₅, R₆, R₇, and R₈ are independently chosen from hydrogen, amino, alkylamino, hydroxyl, halogen (such as chloro and fluoro), C₁-C₄ alkyl (such as methyl), C₁-C₄ haloalkyl (such as trifluoromethyl), C₁-C₄ alkoxy (such as methoxy), C₁-C₄ haloalkoxy and cyano. In some embodiments, R₅, R₆, R₇, and R₈ are methoxy, hydrogen, cyano, or halo (such as Cl, F). In some embodiments, R₅ is amino, alkylamino, trifluoromethyl, hydrogen or halo; R₆ is hydrogen, alkyl (such as methyl) or halo; R₇ is hydrogen, halo, alkyl (such as methyl), alkoxy (such as methoxy), cyano, or trifluoromethyl; and R₈ is hydrogen or halo. In some embodiments, only one of R₅, R₆, R₇, and R₈ is not hydrogen. In some embodiments, R₇ is not hydrogen In some embodiments, R₅, R₆, and R₈ are hydrogen and R₇ is cyano, methoxy or halogen (such as Cl, F).

Certain compounds will be capable of forming acid addition salts (i.e., will comprise a site which reacts with a pharmaceutically acceptable acid to form an acid addition salt.) The present invention includes pharmaceutically acceptable acid addition salts of the compounds of Formula I. Acid addition salts of the present compounds are prepared in a standard manner in a suitable solvent from the parent compound and an excess of an acid, such as hydrochloric, hydrobromic, sulfuric, phosphoric, acetic, maleic, succinic or methanesulfonic. Salt forms include hydrochloric, phosphoric, and oxalic acid salts.

The salts and/or solvates of the compounds of the formula (I) which are not pharmaceutically acceptable may be useful as intermediates in the preparation of pharmaceutically acceptable salts and/or solvates of compounds of formula (I) or the compounds of the formula (I) themselves, and as such form another aspect of the present invention.

In some embodiments, R₁ is benzyl, halobenzyl, methoxybenzyl-, cyanobenzyl, or naphthalenylmethyl-; R₂ is ethyl or propyl; R_{2′} is hydrogen; R₅ is hydrogen; R₆ is hydrogen; R₇ is halo, cyano, methoxy or hydrogen; R₈ is hydrogen; and R₁₂ is —NR₄(COR₃) wherein R₃ is optionally substituted aryl (in some embodiments, halophenyl, halomethylphenyl-, methylenedioxyphenyl-, methoxyphenyl-, ethoxyphenyl-, cyanophenyl- or phenyl substituted with lower-acyl or lower-alkylaminocarbonyl-, e.g. methylaminocarbonyl- or ethylaminocarbonyl-, or di(lower-alkyl)aminocarbonyl-, e.g. dimethylaminocarbonyl- or diethylaminocarbonyl-; and R₄ is R₁₆-alkylene- wherein R₁₆ is hydroxyl, di(C₁-C₄)alkylamino-, (C₁-C₄ alkyl)amino-, amino, pyrrolidino, piperidino, imidazolyl and morpholino (in some embodiments, R₁ is benzyl, halobenzyl, methoxybenzyl, cyanobenzyl, or naphthalenylmethyl; and R₂ is propyl (such asi- or c-propyl).

In some embodiments, R₁ is benzyl, halobenzyl, methoxybenzyl-, cyanobenzyl, or naphthalenylmethyl-; R₂ is ethyl or propyl; R_{2′} is hydrogen; R₅ is hydrogen; R₆ is hydrogen; R₇ is halo, cyano, methoxy or hydrogen; R₈ is hydrogen; R₁₂ is —NR₄(CH₂R_{3b}) wherein R₄ is R₁₆-alkylene- wherein R₁₆ is hydroxyl, di(C₁-C₄)alkylamino-, (C₁-C₄ alkyl)amino-, amino, pyrrolidino, piperidino, imidazolyl or morpholino; and R_{3b} is optionally substituted aryl.

In some embodiments, R₁ is benzyl, halobenzyl, methoxybenzyl, cyanobenzyl, or naphthalenylmethyl; R₂ is chosen from ethyl or propyl; R_{2′} is hydrogen; R₅ is hydrogen; R₆ is hydrogen; R₇ is halo, cyano, methoxy or hydrogen; R₈ is hydrogen; and R₁₂ is optionally substituted imidazolinyl of the above formula wherein R₁₀, R_{10′}, R₁₄ and R_{14′} are independently hydrogen or optionally substituted alkyl (such as optionally substituted C₁-C₄ alkyl); and R₉ is optionally substituted phenyl (such as halophenyl, halomethylphenyl, tolyl, or methylenedioxyphenyl). In some embodiments, R₁ is benzyl, methoxybenzyl, or cyanobenzyl; R₂ is propyl (such as i- or c-propyl); and R₁₆ is amino.

In some embodiments, R₁ is benzyl, halobenzyl, methoxybenzyl, cyanobenzyl, or naphthalenylmethyl; R₂ is chosen from ethyl or propyl; R_{2′} is hydrogen; R₅ is hydrogen; R₆ is hydrogen; R₇ is halo, cyano, methoxy or hydrogen; R₈ is hydrogen; and R₁₂ is optionally substituted imidazole of the above formula wherein R₁₃ is hydrogen and R_{13′} is hydrogen or optionally substituted alkyl (in some embodiments, optionally substituted C₁-C₄ alkyl); and R₉ is optionally substituted aryl (in some embodiments, halophenyl, halomethylphenyl, tolyl, or methylenedioxyphenyl). In some embodiments, R₁₃ is hydrogen and R_{13′} is aminomethyl, aminoethyl aminopropyl, acetylamino-methyl, acetylaminoethyl, benzyloxycarbonylamino-methyl or Benzyloxycarbonylamino-ethyl. In some embodiments, R₁ is benzyl, methoxybenzyl, or cyanobenzyl; R₂ is propyl such as i- or c-propyl); and R₁₆ is amino.

In some embodiments when R₁₂ is —N(R₄)(SO₂R₃ₐ), R₁ is chosen from C₁-C₄ alkyl, benzyl, substituted benzyl and substituted phenyl; R₂ is C₁-C₄ alkyl; R_{2′} is hydrogen; R₃ₐ is chosen from substituted phenyl and naphthyl; R₄ is R₁₆-alkylene-; R₇ is hydrogen, fluoro, methyl or chloro; R₅, R₆ and R₈ are hydrogen; and R₁₆ is chosen from hydroxyl, di(C₁-C₄)amino, (C₁-C₄ alkyl)amino, amino, pyrrolidino, piperidino, imidazolyl and morpholino.

In some embodiments when R₁₂ is —NHR₄ or —N(R₄)(CH₂R_{3b}), R₁ is chosen from hydrogen, optionally substituted C₁-C₄ alkyl, optionally substituted benzyl, optionally substituted phenyl, and optionally substituted naphthalenylmethyl; R₂ is optionally substituted C₁-C₄ alkyl and R_{2′} is hydrogen; R_{3b} is chosen from optionally substituted alkyl; optionally substituted phenyl; biphenylyl, optionally substituted aralkyl; and optionally substituted heterocyclyl; and R₄ is chosen from hydrogen, optionally substituted C₁-C₄ alkyl; cyclohexyl; optionally substituted phenyl; optionally substituted benzyl; heterocyclyl; heteroarylmethyl; heteroarylethyl; and heteroarylpropyl. In some embodiments, R₄ is R₁₆-alkylene-, wherein R₁₆ is hydroxyl, di(C₁-C₄)alkylamino-, (C₁-C₄ alkyl)amino-, amino, C₁-C₄ alkoxy-, or N-heterocyclyl.

In some embodiments, when R₁₂ is —NHR₄ or —N(R₄)(CH₂R_{3b}), R₁ is chosen from C₁-C₄ alkyl, optionally substituted benzyl, and optionally substituted phenyl (in some embodiments, optionally substituted benzyl, e.g., benzyl, cyanobenzyl); R₂ is optionally substituted C₁-C₄ alkyl (in some embodiments, propyl, i- or c-propyl); R_{2′} is hydrogen; R_{3b} is chosen from optionally substituted phenyl, optionally substituted heterocyclyl and naphthyl; R₄ is chosen from hydrogen, optionally substituted benzyl, optionally substituted heterocyclyl and R₁₆-alkylene-; R₆ and R₇ are chosen from halo, cyano, methoxy or hydrogen; R₅ and R₈ are hydrogen; and R₁₆ is chosen from di(C₁-C₄ alkylamino)-, (C₁-C₄ alkyl)amino-, amino, pyrrolidinyl, piperidinyl, imidazolyl and morpholinyl.

In some embodiments, R₁ is benzyl, halobenzyl (such as Cl-benzyl and F-benzyl), methoxybenzyl-, cyanobenzyl, or naphthalenylmethyl-; R₂ is ethyl or propyl; R_{2′} is hydrogen; R₅ is hydrogen; R₆ is hydrogen; R₇ is halo, cyano, methoxy or hydrogen; R₈ is hydrogen; and R₁₂ is —NHR₄ wherein R₄ is hydrogen (in some embodiments, R₁ is benzyl, halobenzyl, cyanobenzyl; and R₂ is propyl, such as i-propyl or c-propyl).

When R_{3b} is present, in some embodiments, it is chosen from phenyl substituted with one or more halo, methyl, methoxy, cyano, trifluoromethyl, trifluoromethoxy, carboxy, and or methoxycarbonyl groups [e.g., halophenyl, polyhalophenyl, tolyl, dimethylphenyl, methoxyphenyl, dimethoxyphenyl, cyanophenyl, trifluoromethylphenyl, trifluoromethoxyphenyl, bis(trifluoromethyl)phenyl, carboxyphenyl, t-butylphenyl, methoxycarbonylphenyl]; piperidinyl and naphthyl.

In some embodiments, the chromenone derivative is chosen from:
N-(3-Amino-propyl)-N-{1-[3-(3-cyano-benzyl)-7-hydroxy-4-oxo-4H-chromen-2-yl]-2-methyl-propyl}-3-fluoro-4-methyl-benzamide;
4-Acetyl-N-(3-amino-propyl)-N-[1-(3-benzyl-7-methoxy-4-oxo-4H-chromen-2-yl)-2-methyl-propyl]-benzamide;
2-{1-[4-(2-Amino-ethyl)-2-p-tolyl-imidazol-1-yl]-2-methyl-propyl}-3-benzyl-4-oxo-4H-chromene-7-carbonitrile;
3-Benzyl-2-[1-(4,4-dimethyl-2-p-tolyl-4,5-dihydro-imidazol-1-yl)-2-methyl-propyl]-4-oxo-4H-chromene-7-carbonitrile;
Benzo[1,3]dioxole-5-carboxylic acid (3-amino-propyl)-{1-[7-chloro-3-(3-cyano-benzyl)-4-oxo-4H-chromen-2-yl]-2-methyl-propyl}-amide;
N-(3-Amino-propyl)-N-{1-[7-chloro-3-(3-cyano-benzyl)-4-oxo-4H-chromen-2-yl]-2-methyl-propyl}-4-methyl-benzamide;
N-(3-Amino-propyl)-N-[1-(7-chloro-3-naphthalen-1-ylmethyl-4-oxo-4H-chromen-2-yl)-2-methyl-propyl]-2-methoxy-acetamide;
4-Acetyl-N-(3-amino-propyl)-N-[1-(3-benzyl-7-cyano-4-oxo-4H-chromen-2-yl)-2-methyl-propyl]-benzamide;
N-(3-Amino-propyl)-N-{1-[7-chloro-3-(3-cyano-benzyl)-4-oxo-4H-chromen-2-yl]-2-methyl-propyl}-3-fluoro-4-methyl-benzamide;
Benzo[1,3]dioxole-5-carboxylic acid (3-amino-propyl)-[1-(3-benzyl-7-cyano-4-oxo-4H-chromen-2-yl)-2-methyl-propyl]-amide;
Benzo[1,3]dioxole-5-carboxylic acid (3-amino-propyl)-[1-(3-benzyl-7-fluoro-4-oxo-4H-chromen-2-yl)-2-methyl-propyl]-amide;
3-Benzyl-2-[1-(4,4-dimethyl-2-p-tolyl-4,5-dihydro-imidazol-1-yl)-2-methyl-propyl]-7-hydroxy-chromen-4-one;
N-(3-Amino-propyl)-N-[1-(3-benzyl-7-fluoro-4-oxo-4H-chromen-2-yl)-2-methyl-propyl]-3-fluoro-4-methyl-benzamide;
N-(3-Amino-propyl)-N-[1-(3-benzyl-7-methoxy-4-oxo-4H-chromen-2-yl)-2-methyl-propyl]-3-fluoro-4-methyl-benzamide;
N-(3-Amino-propyl)-N-[1-(3-benzyl-7-methoxy-4-oxo-4H-chromen-2-yl)-2-methyl-propyl]-4-methyl-benzamide;
3-Benzyl-2-[1-(4,4-dimethyl-2-p-tolyl-4,5-dihydro-imidazol-1-yl)-2-methyl-propyl]-7-methoxy-chromen-4-one;
3-Benzyl-7-fluoro-2-{1-[2-(3-fluoro-4-methyl-phenyl)-4,4-dimethyl-4,5-dihydro-imidazol-1-yl]-2-methyl-propyl}-chromen-4-one;
3-Benzyl-2-[1-(4,4-dimethyl-2-p-tolyl-4,5-dihydro-imidazol-1-yl)-2-methyl-propyl]-7-fluoro-chromen-4-one;
3-Benzyl-2-{1-[2-(3-fluoro-4-methyl-phenyl)-4,4-dimethyl-4,5-dihydro-imidazol-1-yl]-2-methyl-propyl}-7-cyano-chromen-4-one;
N-(3-Amino-propyl)-N-[1-(3-benzyl-7-fluoro-4-oxo-4H-chromen-2-yl)-2-methyl-propyl]-4-methyl-benzamide;
4-Acetyl-N-(3-amino-propyl)-N-[1-(3-benzyl-7-chloro-4-oxo-4H-chromen-2-yl)-2-methyl-propyl]-benzamide;
N-(3-Amino-propyl)-3-fluoro-N-{1-[7-fluoro-3-(3-methoxy-benzyl)-4-oxo-4H-chromen-2-yl]-2-methyl-propyl}-4-methyl-benzamide;
4-Acetyl-N-(3-amino-propyl)-N-{1-[7-chloro-3-(3-cyano-benzyl)-4-oxo-4H-chromen-2-yl]-2-methyl-propyl}-benzamide;
(2-{1-[1-(3-Benzyl-7-chloro-4-oxo-4H-chromen-2-yl)-2-methyl-propyl]-2-p-tolyl-1H-imidazol-4-yl}-ethyl)-carbamic acid benzyl ester;
2-[1-(2-Benzo[1,3]dioxol-5-yl-4,4-dimethyl-4,5-dihydro-imidazol-1-yl)-2-methyl-propyl]-3-benzyl-7-cyano-chromen-4-one;
4-Acetyl-N-(3-amino-propyl)-N-[1-(3-benzyl-7-fluoro-4-oxo-4H-chromen-2-yl)-2-methyl-propyl]-benzamide;
N-(3-Amino-propyl)-N-[1-(3-benzyl-7-chloro-4-oxo-4H-chromen-2-yl)-2-methyl-propyl]-3-fluoro-4-methyl-benzamide;
N-(3-Amino-propyl)-N-{1-[7-fluoro-3-(3-methoxy-benzyl)-4-oxo-4H-chromen-2-yl]-2-methyl-propyl}-4-methoxy-benzamide;
Benzo[1,3]dioxole-5-carboxylic acid (3-amino-propyl)-{1-[7-fluoro-3-(3-methoxy-benzyl)-4-oxo-4H-chromen-2-yl]-2-methyl-propyl}-amide;
2-{1-[4-(2-Amino-ethyl)-2-p-tolyl-imidazol-1-yl]-2-methyl-propyl}-3-benzyl-7-chloro-chromen-4-one;
N-(3-Amino-propyl)-N-[1-(3-benzyl-7-hydroxy-4-oxo-4H-chromen-2-yl)-2-methyl-propyl]-3-fluoro-4-methyl-benzamide;
Benzo[1,3]dioxole-5-carboxylic acid (3-amino-propyl)-[1-(3-benzyl-7-chloro-4-oxo-4H-chromen-2-yl)-2-methyl-propyl]-amide;
N-(3-Amino-propyl)-N-[1-(3-benzyl-7-hydroxy-4-oxo-4H-chromen-2-yl)-2-methyl-propyl]-4-methyl-benzamide;
N-(3-Amino-propyl)-N-{1-[7-fluoro-3-(3-methoxy-benzyl)-4-oxo-4H-chromen-2-yl]2-methyl-propyl}-2-methoxy-acetamide;
N-(3-Amino-propyl)-N-{1-[7-fluoro-3-(3-methoxy-benzyl)-4-oxo-4H-chromen-2-yl]-2-methyl-propyl}-4-methyl-benzamide;
N-(3-Amino-propyl)-N-[(3-benzyl-7-chloro-4-oxo-4H-chromen-2-yl)-cyclopropyl-methyl]-4-methyl-benzamide;
2,3-Dihydro-benzo[1,4]dioxine-6-carboxylic acid (3-amino-propyl)-[1-(3-benzyl-7-chloro-4-oxo-4H-chromen-2-yl)-2-methyl-propyl]-amide;
N-(3-Amino-propyl)-3-fluoro-N-{1-[7-cyano-3-(3-methoxy-benzyl)-4-oxo-4H-chromen-2-yl]-2-methyl-propyl}-4-methyl-benzamide;
3-Benzyl-7-chloro-2-[2-methyl-1-(2-p-tolyl-4,5-dihydro-imidazol-1-yl)-propyl]-chromen-4-one;
3-Benzyl-7-fluoro-2-{1-[2-(3-fluoro-4-methyl-phenyl)-4,5-dihydro-imidazol-1-yl]-2-methyl-propyl}-chromen-4-one;
2-[1-(4-Aminomethyl-2-p-tolyl-imidazol-1-yl)-2-methyl-propyl]-3-benzyl-7-chloro-chromen-4-one;
3-Benzyl-7-methoxy-2-[2-methyl-1-(2-p-tolyl-4,5-dihydro-imidazol-1-yl)-propyl]-chromen-4-one;
2-[1-(2-Benzo[1,3]dioxol-5-yl-4,5-dihydro-imidazol-1-yl)-2-methyl-propyl]-3-benzyl-7-chloro-chromen-4-one;
4-Acetyl-N-(3-amino-propyl)-N-[1-(3-benzyl-7-chloro-4-oxo-4H-chromen-2-yl)-2-methyl-propyl]-benzamide;
4-Acetyl-N-(3-amino-propyl)-N-{1-[7-cyano-3-(3-methoxy-benzyl)-4-oxo-4H-chromen-2-yl]-2-methyl-propyl}-benzamide;
3-Benzyl-7-chloro-2-{1-[2-(3-fluoro-4-methyl-phenyl)-4,5-dihydro-imidazol-1-yl]-2-methyl-propyl}-chromen-4-one;
3-Benzyl-7-fluoro-2-[2-methyl-1-(2-p-tolyl-4,5-dihydro-imidazol-1-yl)-propyl]-chromen-4-one;
2-[1-(2-Benzo[1,3]dioxol-5-yl-4,5-dihydro-imidazol-1-yl)-2-methyl-propyl]-3-benzyl-7-fluoro-chromen-4-one;
N-(3-Amino-propyl)-N-{1-[7-cyano-3-(3-methoxy-benzyl)-4-oxo-4H-chromen-2-yl]-2-methyl-propyl}-4-methyl-benzamide;
Benzo[1,3]dioxole-5-carboxylic acid (3-amino-propyl)-{1-[7-cyano-3-(3-methoxy-benzyl)-4-oxo-4H-chromen-2-yl]-2-methyl-propyl}-amide;
3-(2-{1-[2-(3-Fluoro-4-methyl-phenyl)-4,5-dihydro-imidazol-1-yl]-2-methyl-propyl}-7-methoxy-4-oxo-4H-chromen-3-ylmethyl)-benzonitrile;
3-{7-Chloro-2-[2-methyl-1-(2-p-tolyl-4,5-dihydro-imidazol-1-yl)-propyl]-4-oxo-4H-chromen-3-ylmethyl}-benzonitrile;
3-{7-Methoxy-2-[2-methyl-1-(2-p-tolyl-4,5-dihydro-imidazol-1-yl)-propyl]-4-oxo-4H-chromen-3-ylmethyl}-benzonitrile;
3-{7-Fluoro-2-[2-methyl-1-(2-p-tolyl-4,5-dihydro-imidazol-1-yl)-propyl]-4-oxo-4H-chromen-3-ylmethyl}-benzonitrile;
N-(3-Amino-propyl)-N-{1-[7-chloro-3-(3-methoxy-benzyl)-4-oxo-4H-chromen-2-yl]-2-methyl-propyl}-3-fluoro-4-methyl-benzamide;
N-(3-Amino-propyl)-N-{1-[3-(3-cyano-benzyl)-7-methoxy-4-oxo-4H-chromen-2-yl]-2-methyl-propyl}-4-methyl-benzamide;
N-(3-Amino-propyl)-N-{1-[3-(3-cyano-benzyl)-7-methoxy-4-oxo-4H-chromen-2-yl]-2-methyl-propyl}-3-fluoro-4-methyl-benzamide;
3-{2-[1-(2-Benzo[1,3]dioxol-5-yl-4,5-dihydro-imidazol-1-yl)-2-methyl-propyl]-7-fluoro-4-oxo-4H-chromen-3-ylmethyl}-benzonitrile;
3-Benzyl-2-{1-[2-(3-fluoro-4-methyl-phenyl)-4,5-dihydro-imidazol-1-yl]-2-methyl-propyl}-4-oxo-4H-chromene-7-carbonitrile;
2-[1-(2-Benzo[1,3]dioxol-5-yl-4,5-dihydro-imidazol-1-yl)-2-methyl-propyl]-3-benzyl-4-oxo-4H-chromene-7-carbonitrile;
3-(7-Chloro-2-{1-[2-(3-fluoro-4-methyl-phenyl)-4,5-dihydro-imidazol-1-yl]-2-methyl-propyl}-4-oxo-4H-chromen-3-ylmethyl)-benzonitrile;
3-{2-[1-(2-Benzo[1,3]dioxol-5-yl-4,5-dihydro-imidazol-1-yl)-2-methyl-propyl]-7-methoxy-4-oxo-4H-chromen-3-ylmethyl}-benzonitrile;
3-(7-Fluoro-2-{1-[2-(3-fluoro-4-methyl-phenyl)-4,5-dihydro-imidazol-1-yl]-2-methyl-propyl}-4-oxo-4H-chromen-3-ylmethyl)-benzonitrile;
Benzo[1,3]dioxole-5-carboxylic acid (3-amino-propyl)-{1-[7-cyano-3-(3-cyano-benzyl)-4-oxo-4H-chromen-2-yl]-2-methyl-propyl}-amide;
Benzo[1,3]dioxole-5-carboxylic acid (3-amino-propyl)-{1-[3-(3-cyano-benzyl)-7-methoxy-4-oxo-4H-chromen-2-yl]-2-methyl-propyl}-amide;
Benzo[1,3]dioxole-5-carboxylic acid (3-amino-propyl)-{1-[3-(3-cyano-benzyl)-7-fluoro-4-oxo-4H-chromen-2-yl]-2-methyl-propyl}-amide;
N-(3-Amino-propyl)-N-{1-[3-(3-cyano-benzyl)-7-fluoro-4-oxo-4H-chromen-2-yl]-2-methyl-propyl}-4-methyl-benzamide;
Benzo[1,3]dioxole-5-carboxylic acid (3-amino-propyl)-{1-[7-chloro-3-(3-methoxy-benzyl)-4-oxo-4H-chromen-2-yl]-2-methyl-propyl}-amide;
N-(3-Amino-propyl)-N-{1-[7-cyano-3-(3-cyano-benzyl)-4-oxo-4H-chromen-2-yl]-2-methyl-propyl}-4-methyl-benzamide;
3-Benzyl-2-[2-methyl-1-(2-p-tolyl-4,5-dihydro-imidazol-1-yl)-propyl]-4-oxo-4H-chromene-7-carbonitrile;
2-[1-(2-Benzo[1,3]dioxol-5-yl-4,5-dihydro-imidazol-1-yl)-2-methyl-propyl]-3-benzyl-7-methoxy-chromen-4-one;
N-(3-Amino-propyl)-N-{1-[7-chloro-3-(3-methoxy-benzyl)-4-oxo-4H-chromen-2-yl]-2-methyl-propyl}-2-methoxy-acetamide;
N-(3-Amino-propyl)-N-{1-[7-cyano-3-(3-cyano-benzyl)-4-oxo-4H-chromen-2-yl]-2-methyl-propyl}-3-fluoro-4-methyl-benzamide;
N-(3-Amino-propyl)-N-{1-[3-(3-cyano-benzyl)-7-fluoro-4-oxo-4H-chromen-2-yl]-2-methyl-propyl}-3-fluoro-4-methyl-benzamide;
2-{1-[4-(2-Amino-ethyl)-2-p-tolyl-imidazol-1-yl]-2-methyl-propyl}-7-chloro-3-(3-methoxy-benzyl)-chromen-4-one;
2-{1-[4-(2-Amino-ethyl)-2-(3-fluoro-4-methyl-phenyl)-imidazol-1-yl]-2-methyl-propyl}-3-benzyl-7-fluoro-chromen-4-one;
2-{1-[4-(2-Amino-ethyl)-2-(3-fluoro-4-methyl-phenyl)-imidazol-1-yl]-2-methyl-propyl}-3-benzyl-4-oxo-4H-chromene-7-carbonitrile;
2-{1-[4-(2-Amino-ethyl)-2-p-tolyl-imidazol-1-yl]-2-methyl-propyl}-3-benzyl-7-fluoro-chromen-4-one;
2-{1-[4-(2-Amino-ethyl)-2-(3-fluoro-4-methyl-phenyl)-imidazol-1-yl]-2-methyl-propyl}-7-chloro-3-(3-methoxy-benzyl)-chromen-4-one;
2-{1-[4-(2-Amino-ethyl)-2-(3-fluoro-4-methyl-phenyl)-imidazol-1-yl]-2-methyl-propyl}-3-benzyl-7-chloro-chromen-4-one;
3-Benzyl-2-{1-[2-(3-fluoro-4-methyl-phenyl)-4,5-dihydro-imidazol-1-yl]-2-methyl-propyl}-7-methoxy-chromen-4-one;
3-(2-{1-[4-(2-Amino-ethyl)-2-p-tolyl-imidazol-1-yl]-2-methyl-propyl}-7-chloro-4-oxo-4H-chromen-3-ylmethyl)-benzonitrile;
3-(2-{1-[4-(2-Amino-ethyl)-2-(3-fluoro-4-methyl-phenyl)-imidazol-1-yl]-2-methyl-propyl}-7-chloro-4-oxo-4H-chromen-3-ylmethyl)-benzonitrile;
2-{1-[4-(2-Amino-ethyl)-2-p-tolyl-imidazol-1-yl]-2-methyl-propyl}-7-fluoro-3-(3-methoxy-benzyl)-chromen-4-one;
2-{1-[4-(2-Amino-ethyl)-2-(3-fluoro-4-methyl-phenyl)-imidazol-1-yl]-2-methyl-propyl}-3-(3-cyano-benzyl)-4-oxo-4H-chromene-7-carbonitrile;
N-(3-Amino-propyl)-3-fluoro-N-{1-[7-hydroxy-3-(3-methoxy-benzyl)-4-oxo-4H-chromen-2-yl]-2-methyl-propyl}-4-methyl-benzamide;
2-{1-[4-(2-Amino-ethyl)-2-p-tolyl-imidazol-1-yl]-2-methyl-propyl}-3-(3-methoxy-benzyl)-4-oxo-4H-chromene-7-carbonitrile;
N-(3-Amino-propyl)-N-[1-(3-benzyl-7-cyano-4-oxo-4H-chromen-2-yl)-2-methyl-propyl]-4-ethoxy-benzamide;
N-(3-Amino-propyl)-N-[1-(3-benzyl-7-cyano-4-oxo-4H-chromen-2-yl)-2-methyl-propyl]-6-trifluoromethyl-nicotinamide;
N-(3-Amino-propyl)-N-[1-(3-benzyl-7-fluoro-4-oxo-4H-chromen-2-yl)-2-methyl-propyl]-6-trifluoromethyl-nicotinamide;
N-(3-Amino-propyl)-N-[1-(3-benzyl-7-fluoro-4-oxo-4H-chromen-2-yl)-2-methyl-propyl]-isonicotinamide;
N-(3-Amino-propyl)-N-[1-(3-benzyl-7-chloro-4-oxo-4H-chromen-2-yl)-2-methyl-propyl]-4-cyano-benzamide;
4-Acetylamino-N-(3-amino-propyl)-N-[1-(3-benzyl-7-chloro-4-oxo-4H-chromen-2-yl)-2-methyl-propyl]-benzamide;
N-(3-Amino-propyl)-N-[1-(3-benzyl-7-chloro-4-oxo-4H-chromen-2-yl)-2-methyl-propyl]-6-trifluoromethyl-nicotinamide;
Benzo[1,2,3]thiadiazole-5-carboxylic acid (3-amino-propyl)-[1-(3-benzyl-7-chloro-4-oxo-4H-chromen-2-yl)-2-methyl-propyl]-amide;
Benzo[1,2,3]thiadiazole-5-carboxylic acid (3-amino-propyl)-[1-(3-benzyl-7-fluoro-4-oxo-4H-chromen-2-yl)-2-methyl-propyl]-amide;
4-Acetylamino-N-(3-amino-propyl)-N-[1-(3-benzyl-7-fluoro-4-oxo-4H-chromen-2-yl)-2-methyl-propyl]-benzamide;
Benzo[1,2,3]thiadiazole-5-carboxylic acid (3-amino-propyl)-[1-(3-benzyl-7-fluoro-4-oxo-4H-chromen-2-yl)-2-methyl-propyl]-amide;
N-(3-Amino-propyl)-N-[1-(3-benzyl-7-chloro-4-oxo-4H-chromen-2-yl)-2-methyl-propyl]-nicotinamide;
N-(3-Amino-propyl)-N-[1-(3-benzyl-7-fluoro-4-oxo-4H-chromen-2-yl)-2-methyl-propyl]-4-methoxy-benzamide;
Benzo[1,2,3]thiadiazole-5-carboxylic acid (3-amino-propyl)-[1-(3-benzyl-7-cyano-4-oxo-4H-chromen-2-yl)-2-methyl-propyl]-amide;
5-Methyl-pyrazine-2-carboxylic acid (3-amino-propyl)-[1-(3-benzyl-7-chloro-4-oxo-4H-chromen-2-yl)-2-methyl-propyl]-amide;
N-(3-Amino-propyl)-N-[1-(3-benzyl-7-cyano-4-oxo-4H-chromen-2-yl)-2-methyl-propyl]-3-dimethylamino-benzamide;
2-[1-(2-Benzo[1,3]dioxol-5-yl-4,5-dihydro-imidazol-1-yl)-2-methyl-propyl]-3-(3-methoxy-benzyl)-4-oxo-4H-chromene-7-carbonitrile;
7-Chloro-3-(3-methoxy-benzyl)-2-[2-methyl-1-(2-p-tolyl-4,5-dihydro-imidazol-1-yl)-propyl]-chromen-4-one;
7-Chloro-2-{1-[2-(3-fluoro-4-methyl-phenyl)-4,5-dihydro-imidazol-1-yl]-2-methyl-propyl}-3-(3-methoxy-benzyl)-chromen-4-one;
2-[1-(2-Benzo[1,3]dioxol-5-yl-4,5-dihydro-imidazol-1-yl)-2-methyl-propyl]-7-chloro-3-(3-methoxy-benzyl)-chromen-4-one;
7-Fluoro-3-(3-methoxy-benzyl)-2-[2-methyl-1-(2-p-tolyl-4,5-dihydro-imidazol-1-yl)-propyl]-chromen-4-one;
7-Fluoro-2-{1-[2-(3-fluoro-4-methyl-phenyl)-4,5-dihydro-imidazol-1-yl]-2-methyl-propyl}-3-(3-methoxy-benzyl)-chromen-4-one;
2-{1-[4-(2-Amino-ethyl)-2-(3-fluoro-4-methyl-phenyl)-imidazol-1-yl]-2-methyl-propyl}-7-fluoro-3-(3-methoxy-benzyl)-chromen-4-one;
2-{1-[4-(2-Acetylamino-ethyl)-2-(3-fluoro-4-methyl-phenyl)-imidazol-1-yl]-2-methyl-propyl}-3-(3-methoxy-benzyl)-4-oxo-4H-chromene-7-carboxylic acid amide;
3-(2-{1-[4-(2-Amino-ethyl)-2-(3-fluoro-4-methyl-phenyl)-imidazol-1-yl]-2-methyl-propyl}-7-fluoro-4-oxo-4H-chromen-3-ylmethyl)-benzonitrile;
N-{1-[1-(3-Benzyl-7-chloro-4-oxo-4H-chromen-2-yl)-2-methyl-propyl]-2-p-tolyl-1H-imidazol-4-ylmethyl}-acetamide;
Benzo[b]thiophene-2-carboxylic acid (3-amino-propyl)-[1-(3-benzyl-7-cyano-4-oxo-4H-chromen-2-yl)-2-methyl-propyl]-amide;
1-Methyl-1H-indole-2-carboxylic acid (3-amino-propyl)-[1-(3-benzyl-7-cyano-4-oxo-4H-chromen-2-yl)-2-methyl-propyl]-amide;
5-tert-Butyl-2-methyl-2H-pyrazole-3-carboxylic acid (3-amino-propyl)-[1-(3-benzyl-7-cyano-4-oxo-4H-chromen-2-yl)-2-methyl-propyl]-amide;
2,5-Dimethyl-2H-pyrazole-3-carboxylic acid (3-amino-propyl)-[1-(3-benzyl-7-cyano-4-oxo-4H-chromen-2-yl)-2-methyl-propyl]-amide;
5-tert-Butyl-2-methyl-2H-pyrazole-3-carboxylic acid (3-amino-propyl)-[1-(3-benzyl-7-fluoro-4-oxo-4H-chromen-2-yl)-2-methyl-propyl]-amide;
2,5-Dimethyl-2H-pyrazole-3-carboxylic acid (3-amino-propyl)-[1-(3-benzyl-7-chloro-4-oxo-4H-chromen-2-yl)-2-methyl-propyl]-amide;
2,5-Dimethyl-2H-pyrazole-3-carboxylic acid (3-amino-propyl)-[1-(3-benzyl-7-fluoro-4-oxo-4H-chromen-2-yl)-2-methyl-propyl]-amide;
5-Methyl-2H-pyrazole-3-carboxylic acid (3-amino-propyl)-[1-(3-benzyl-7-cyano-4-oxo-4H-chromen-2-yl)-2-methyl-propyl]-amide;
5-Methyl-2H-pyrazole-3-carboxylic acid (3-amino-propyl)-[1-(3-benzyl-7-fluoro-4-oxo-4H-chromen-2-yl)-2-methyl-propyl]-amide;
Furan-2-carboxylic acid (3-amino-propyl)-[1-(3-benzyl-7-fluoro-4-oxo-4H-chromen-2-yl)-2-methyl-propyl]-amide;
Furan-2-carboxylic acid (3-amino-propyl)-[1-(3-benzyl-7-chloro-4-oxo-4H-chromen-2-yl)-2-methyl-propyl]-amide;
2,5-Dimethyl-furan-3-carboxylic acid (3-amino-propyl)-[1-(3-benzyl-7-cyano-4-oxo-4H-chromen-2-yl)-2-methyl-propyl]-amide;
2,5-Dimethyl-furan-3-carboxylic acid (3-amino-propyl)-[1-(3-benzyl-7-fluoro-4-oxo-4H-chromen-2-yl)-2-methyl-propyl]-amide;
5-Methyl-thiophene-2-carboxylic acid (3-amino-propyl)-[1-(3-benzyl-7-cyano-4-oxo-4H-chromen-2-yl)-2-methyl-propyl]-amide;
5-Methyl-thiophene-2-carboxylic acid (3-amino-propyl)-[1-(3-benzyl-7-fluoro-4-oxo-4H-chromen-2-yl)-2-methyl-propyl]-amide;
5-Methyl-isoxazole-3-carboxylic acid (3-amino-propyl)-[1-(3-benzyl-7-chloro-4-oxo-4H-chromen-2-yl)-2-methyl-propyl]-amide;
5-Methyl-2-trifluoromethyl-furan-3-carboxylic acid (3-amino-propyl)-[1-(3-benzyl-7-cyano-4-oxo-4H-chromen-2-yl)-2-methyl-propyl]-amide;
5-Methyl-isoxazole-3-carboxylic acid (3-amino-propyl)-[1-(3-benzyl-7-fluoro-4-oxo-4H-chromen-2-yl)-2-methyl-propyl]-amide;
5-Methyl-isoxazole-3-carboxylic acid (3-amino-propyl)-[1-(3-benzyl-7-cyano-4-oxo-4H-chromen-2-yl)-2-methyl-propyl]-amide;
Benzo[c]isoxazole-3-carboxylic acid (3-amino-propyl)-[1-(3-benzyl-7-chloro-4-oxo-4H-chromen-2-yl)-2-methyl-propyl]-amide;
Benzo[c]isoxazole-3-carboxylic acid (3-amino-propyl)-[1-(3-benzyl-7-fluoro-4-oxo-4H-chromen-2-yl)-2-methyl-propyl]-amide;
1-Methyl-1H-pyrrole-2-carboxylic acid (3-amino-propyl)-[1-(3-benzyl-7-fluoro-4-oxo-4H-chromen-2-yl)-2-methyl-propyl]-amide;
1-Methyl-1H-imidazole-4-carboxylic acid (3-amino-propyl)-[(R)-1-(3-benzyl-7-cyano-4-oxo-4H-chromen-2-yl)-2-methyl-propyl]-amide;
N-(3-Amino-propyl)-N—[(R)-1-(3-benzyl-7-cyano-4-oxo-4H-chromen-2-yl)-2-methyl-propyl]-3-dimethylamino-benzamide;
5-Methyl-2-trifluoromethyl-furan-3-carboxylic acid (3-amino-propyl)-[(R)-1-(3-benzyl-7-cyano-4-oxo-4H-chromen-2-yl)-2-methyl-propyl]-amide;
5-Methyl-isoxazole-3-carboxylic acid (3-amino-propyl)-[(R)-1-(3-benzyl-7-cyano-4-oxo-4H-chromen-2-yl)-2-methyl-propyl]-amide;
1-Methyl-1H-imidazole-4-carboxylic acid (3-amino-propyl)-[(R)-1-(3-benzyl-7-fluoro-4-oxo-4H-chromen-2-yl)-2-methyl-propyl]-amide;
1-Methyl-1H-pyrrole-2-carboxylic acid (3-amino-propyl)-[(R)-1-(3-benzyl-7-fluoro-4-oxo-4H-chromen-2-yl)-2-methyl-propyl]-amide;
Benzo[c]isoxazole-3-carboxylic acid (3-amino-propyl)-[(R)-1-(3-benzyl-7-fluoro-4-oxo-4H-chromen-2-yl)-2-methyl-propyl]-amide; and
5-Methyl-isoxazole-3-carboxylic acid (3-amino-propyl)-[(R)-1-(3-benzyl-7-fluoro-4-oxo-4H-chromen-2-yl)-2-methyl-propyl]-amide, and

pharmaceutically acceptable salts thereof.

All compound forms suitable for use in the present invention, which include starting materials, intermediates or products, etc., and/or corresponding pharmaceutical compositions, are prepared as described herein, and/or by the application or adaptation of known methods, which may be methods used heretofore or as described in the literature.

Examples of chromenone compounds synthesized via conventional organic chemical techniques known in the art. See, for example, U.S. Pat. No. 6,924,376, which is incorporated herein by reference.

Optically active (R)- and (S)-isomers may be prepared using chiral synthons or chiral reagents, or resolved using conventional techniques. When the compounds described herein contain alkenyl or olefinic double bonds (i.e., such as configurations with centers of geometric asymmetry) and unless specified otherwise, it is intended that compounds containing such geometric configurations, may include both E and Z geometric isomers. Likewise, all tautomeric forms of such isomers also are encompassed by the present invention.

Also, in accordance with the present invention, when desired, chromenone compounds as described herein with R- and/or S-isomer forms may be resolved by methods known to those skilled in the art, for example by formation of diastereoisomeric salts or complexes which may be separated, for example, by crystallisation; via formation of diastereoisomeric derivatives which may be separated, for example, by crystallisation, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that where the desired enantiomer is converted into another chemical entity by one of the separation procedures described above, a further step may be required to liberate the desired enantiomeric form. Alternatively, specific enantiomer may be synthesized by asymmetric synthesis using optically active reagents, substrates, catalysts or solvents, or by converting on enantiomer to the other by symmetric transformation.

The methods and compositions of the invention further utilize a chemotherapeutic agent in addition to the chromenone derivative.

Suitable chemotherapeutic agents for use in accordance with the present invention include:

neutropenia treatment agents (e.g., which may include one or more hematopoietic growth factors which regulates the production and function of neutrophils such as a human granulocyte colony stimulating factor, (G-CSF), such as filgrastim);

alkylating agents (e.g., which may include doxorubicin, cyclophosphamide, estramustine, carmustine, mitomycin, bleomycin and the like);

antimetabolites (e.g., which may include 5-Fluoro-Uracil, capecitabine, gemcitabine, nelarabine, fludarabine, methotrexate and the like);

platinating agents (e.g., which may include cisplatin, oxaliplatin, carboplatin and the like);

topoisomerase inhibitors (e.g., which may include topotecan, irinotecan, etoposide and the like);

tubulin agents (e.g., which may include paclitaxel, docetaxel, vinorelbine, vinblastine, vincristine, other taxanes, epothilones, and the like);

signalling inhibitors (e.g., kinase inhibitors, antibodies, farnesyltransferase inhibitors, in some embodiments kinase inhibitors) (e.g., which may include Herceptin® (trastuzumab), Gleevec®) (imatinib mesylate), Irressa® (gefitinib), Tarceva™ (erlotinib), avastin, Erbitux™ (cetuximab) and the like);

proteasome inhibitors (e.g., Velcade®) (bortezomib); investigational new drug PR-171 from Proteolix; and/or

other chemotherapeutic agents (e.g, which may include tamoxifen, anti-mitotic agents such as polo-like kinase inhibitors or aurora kinase inhibitors, and the like).

In one embodiment, the chemotherapeutic agent is selected from neutropenia treatment agents, alkylating agents, antimetabolites, platinating agents, tubulin agents, topoisomerase inhibitors, signaling inhibitors, and proteasome inhibitors. In another embodiment, the chemotherapeutic agent is selected from neutropenia treatment agents, alkylating agents, antimetabolites, platinating agents, tubulin agents, topoisomerase inhibitors, and proteasome inhibitors. In another embodiment, the chemotherapeutic agent is selected from neutropenia treatment agents, alkylating agents, antimetabolites, platinating agents, and proteasome inhibitors.

In one embodiment, the chemotherapeutic agent is selected from G-CSF, doxorubucin, cisplatin, 5-fluoruracil, gemcitabine, irinotecan, docetaxel, capecitabine, carboplatin, and bortezomib.

In another embodiment, the chemotherapeutic agent is selected from doxorubucin, cisplatin, 5-fluoruracil, gemcitabine, capecitabine, carboplatin, and bortezomib.

Combinations of such types of agents, including one or more of such types of agents (e.g., two platinating agents, a platinating agent and a tubulin agent, etc.), may be used herein.

In addition, active agents and/or pharmaceutical compositions of the invention may be administered alone or in combination with other treatments, e.g., radiation.

The present invention relates to pharmaceutical compositions, comprising:

[a] a chromenone derivative as described herein, including but not limited to each express embodiment;

[b] a chemotherapeutic agent selected from neutropenia treatment agents, alkylating agents, antimetabolites, platinating agents; topoisomerase inhibitors, tubulin agents, signalling inhibitors, proteasome inhibitors, and other chemotherapeutic agents, such as described herein, including but not limited to each express embodiment; and optionally

[c] a pharmaceutically acceptable excipient.

Depending upon the manner of introduction, the compounds may be components in a pharmaceutical composition or formulated in a variety of ways as discussed below.

Pharmaceutical compositions of the present invention generally are prepared using conventional art known materials and techniques, which may include, but are not limited to mixing, blending and the like.

One or more excipients may be used. Suitable excipients contemplated for use in pharmaceutical compositions of the present invention may include those known in the pharmaceutical formulary arts. For example, a reference to useful materials may be found in well-known pharmaceutical formulary compilation text books, such as Remington's Pharmaceutical Sciences, Mack Publishing Co., Easton, Pa. (e.g., 20^{th} Ed., 2000), and Handbook of Pharmaceutical Excipients, American Pharmaceutical Association, Washington, D.C., (e.g., 1st, 2^{nd} and 3^{rd} Eds., 1986, 1994 and 2000, respectively). Such excipients may be employed to prepare compositions acceptable or adaptable for human use. As will be understood by those skilled in the art, various excipients may provide a variety of functions and may be described, among other things, as adjuvants, carriers, diluents, etc.

For example, pharmaceutical compositions of the present invention may include ingredients such as stabilizers, antioxidants, liposomes, preservatives, lubricants, suspending agents, viscosity modifiers and the like, provided that the ingredients do not have a detrimental effect on the therapeutic action of the instant compositions.

Similarly, excipients suitable for use in the present invention may include time delay materials well known in the art, such as glyceryl monostearate or glyceryl distearate alone or with a wax, ethylcellulose, hydroxypropylmethylcellulose, methylmethacrylate and the like.

Treatment regimens for the administration of the compounds and/or compositions of the present invention may be determined readily by those with ordinary skill in art.

The compounds and/or compositions of the invention are administered to mammals and mammalian cells. As used herein, “cells” means cells in which mitosis or meiosis can be altered.

A “patient” for the purposes of the present invention includes both humans and other animals, particularly mammals, and other organisms. Thus the methods are applicable to both human therapy and veterinary applications. In the preferred embodiment the patient is a mammal, and in the most preferred embodiment the patient is human.

While individual needs vary, determination of optimal ranges of effective amounts of each component is within the skill of the art.

Moreover, optimal dosages for a specific pathological condition in a particular patient may ascertained by those of ordinary skill in the art using conventional dosage determination tests in view of the experimental data.

Moreover, the quantity of the compounds and/or pharmaceutical compositions within the present invention as administered will vary over a wide range based upon each individual patient, such that a unit dosage provided is in an effective amount based upon patient body weight or surface area, administration mode per day to achieve the desired effect, etc. (i.e., which may be in any effective amount to achieve the desired effect).

In accordance with the present invention, the term “effective amount” means that amount of a compound and/or corresponding pharmaceutical composition, upon administration to a mammal (such as a human being), in need thereof provides a clinically desirable result in the treatment of cellular proliferative diseases as described herein.

By “therapeutically effective dose” herein is meant a dose that produces the effects for which it is administered.

By “administered” herein is meant administration of a therapeutically effective dose of the compounds of the invention (i.e., the chromenone derivative and/or other chemotherapeutic agent such as described herein) (including in the form of a composition thereof) to a cell either in cell culture or in a patient.

An exact therapeutically effective dose will depend on the purpose of the treatment, and will be ascertainable by one skilled in the art using known techniques.

As is known in the art, adjustments for systemic versus localized delivery, age, body weight, general health, sex, diet, time of administration, drug interaction and the severity of the condition may be necessary, and will be ascertainable with routine experimentation by those skilled in the art.

In light of this, it will be appreciated that the actual course of therapy will vary according to, inter alia, the mode of administration, the particular formulation of the compounds being utilized, the mode of administration and the particular host being treated.

Further, it will be appreciated that the actual dosages of the compound(s) used in the compositions and methods of treatment of the present invention will vary according to the particular compound species or complex being used, the particular composition formulated, the mode of administration and the particular site, such as host and tumor type being treated, etc.

In accordance with the present invention, compounds having the desired pharmacological activity may be administered in a physiologically acceptable carrier to a patient, as described herein. Components of the pharmaceutical composition(s) will depend upon the treatment effected and/or intended route of administration.

The percentage of active compounds in pharmaceutical compositions of the present invention may be varied for a desired amount of active compound in such therapeutically useful compositions such that a suitable dosage will be obtained.

Compounds, pharmaceutical compositions and/or methods within the scope of this invention include all compounds, pharmaceutical compositions, and corresponding treatment methods, wherein the aforementioned compounds of the present invention may be contained in an amount effective to achieve its intended purpose.

For example, the concentration of therapeutically active compound in the formulation may vary from about 0.1 wt. % to about 100 wt. %.

The administration of the active agents, such as compounds and compositions of the present invention can be done in a variety of ways as discussed above, including, but not limited to, orally, subcutaneously, intravenously, intranasally, transdermally, intraperitoneally, intramuscularly, intrapulmonary, vaginally, rectally, or intraocularly. In some instances, for example, in the treatment of wounds and inflammation, the anti-mitotic agents may be directly applied as a solution or spray.

The compounds and/or pharmaceutical compositions of the present invention may also be administered in injectable dosages by solution or suspension of these materials in a physiologically acceptable diluent with pharmaceutical excipients.

For example, sterile liquids, such as water and oils, with or without the addition of a surfactant and other pharmaceutically and physiologically acceptable carrier, including other excipients stabilizers, etc., may be used. Under ordinary conditions of storage and use, these preparations contain a preservative to prevent the growth of microorganisms.

Suitable oils for use in the present invention may include, but are not limited to petroleum, animal, vegetable, or synthetic origin, for example, peanut oil, soybean oil, or mineral oil, and the like.

In general, liquid carriers, particularly for injectable solutions, may include, but are not limited to, water, saline, aqueous dextrose and related sugar solution, and glycols, such as propylene glycol or polyethylene glycol, and the like.

The pharmaceutical forms of the present invention suitable for injectable use, may include, but are not limited to, sterile aqueous solutions or dispersions and sterile powders for extemporaneous preparation of sterile injectable solutions or dispersions and the like. In all cases, each form should be sterile and be fluid to the extent that easy syringability exists.

Such forms should be stable under conditions of manufacture and storage, which should be preserved against contaminating action of microorganisms, such as bacteria and fungi. For example, a carrier may be a solvent or dispersion medium which may include, but are not limited to water, ethanol, polyol (e.g., glycerol, propylene glycol, and liquid polyethylene glycol), vegetable oils, suitable mixtures thereof, and the like.

For parenteral administration, a pharmaceutical composition of the present invention may include, but is not limited to be in the form of a sterile injectable liquid, such as an ampule or an aqueous or nonaqueous liquid suspension, and the like. Suitable solutions or suspensions of active compounds of the present invention may be prepared in water suitably mixed with a surfactant, such as hydroxypropylcellulose. Suitable dispersions may be prepared in, e.g., glycerol, liquid polyethylene glycols, and oil mixtures thereof, and the like.

Moreover, a wide variety of pharmaceutical forms may be employed for use with the present invention.

In light of the foregoing, excipients used in forming pharmaceutical compositions of the present invention may be either a solid (i.e., such as in tablets, capsules, powders, etc.) or liquid form (i.e., such as in solutions, suspensions, or emulsions, etc.)

For example, if a solid carrier is used, the preparation may be, e.g., tableted, placed in a hard gelatin capsule in powder or pellet form, or in the form of a troche or lozenge.

If a liquid carrier is used, the preparation may be, e.g, in the form of a syrup, emulsion, soft gelatin capsule, sterile injectable solution or suspension in an ampule or vial or nonaqueous liquid suspension. For example, to obtain a stable water-soluble dose form, a pharmaceutically acceptable salt of the compound of Formula I may be dissolved in an aqueous solution, e.g., of an organic or inorganic acid or base. If a soluble salt form is not available, the compound of Formula I may be dissolved in a suitable co-solvent or combinations thereof.

Examples of such suitable co-solvents include, but are not limited to, alcohol, propylene glycol, polyethylene glycol 300, polysorbate 80, glycerin and the like in concentrations ranging from 0-60% of the total volume.

Moreover, if desired a pharmaceutical composition is employed in the form of a solution or suspension.

Examples of appropriate pharmaceutical carriers or diluents for solutions or suspensions, may be, liquid, solid, or aerosol, and aqueous or nonaqueous. For example, pharmaceutical carriers or diluents for solutions or suspensions include water, ethanol, glycerin, propylene glycol, olive oil, corn oil, cottonseed oil, peanut oil, sesame oil, liquid paraffins, and mixtures thereof with water; for solid systems: lactose, terra alba, sucrose, talc, gelatin, agar, pectin, acacia, magnesium stearate, stearic acid, kaolin and mannitol; and for aerosol systems: dichlorodifluoromethane, chlorotrifluoroethane and compressed carbon dioxide.

For topical administration, a compound and/or pharmaceutical composition of the present invention may be, e.g., in the form of a cream, ointment, liniment, lotion, paste, spray or drops suitable for administration to the skin, eye, ear, nose or genitalia and the like.

For oral administration, a compound and/or pharmaceutical composition of the present invention may be, e.g., in the form of a tablet, capsule, powder, pellet, troche, lozenge, syrup, suspension, elixir, liquid, or emulsion and/or other solid unit dosage forms as conventionally known in the art and the like.

For example, active compounds and/or pharmaceutical compositions of the present invention may be orally administered with an inert diluent, an assimilable edible carrier, enclosed in hard or soft-shell capsules, compressed into tablets, and/or incorporated directly with food, etc.

A solid form suitable for use in the present invention may include, e.g., lubricants, inert fillers (i.e., such as, lactose, sucrose, or cornstarch, etc.) and the like, etc. When the dosage unit form is a capsule (e.g., an ordinary gelatin type), it also may contain a solid or liquid carrier, e.g, a liquid carrier such as a fatty oil, etc.

In another embodiment, these active compounds and/or pharmaceutical compositions thereof may be tableted with conventional tablet bases, which may include, e.g., lactose, sucrose, or cornstarch and the like, in combination with binders (e.g., acacia, gum, tragacanth, cornstarch, or gelatin, etc.); disintegrating agents (e.g., cornstarch, potato starch, or alginic acid); lubricants (e.g., stearic acid, magnesium stearate, etc.); sweetening agents (e.g., sucrose, lactose, or saccharin, etc.) and/or other excipients (e.g., dicalcium phosphate).

Various other materials may be present as coatings or to modify physical forms of each dosage unit associated with the present invention.

For instance, tablets may be coated with materials, which may include, but are not limited to shellac and/or, sugar, a syrup (i.e., which may include, but is not limited to an active ingredient, a sweetening agent (i.e., such as sucrose), preservatives (i.e., such as methyl and propylparabens), a dye, and flavorings (i.e., such as cherry or orange flavors), and the like.

In one embodiment, the present invention relates to a pharmaceutical composition, which comprises:

[a] a compound of Formula I or a pharmaceutically acceptable salt thereof, as defined herein;

[b] a chemotherapeutic agent selected from neutropenia treatment agents, alkylating agents, antimetabolites, platinating agents; topoisomerase inhibitors, tubulin agents, signalling inhibitors (e.g., kinase inhibitors), and proteasome inhibitors; and optionally

[c] a pharmaceutically acceptable excipient.

In one embodiment, the pharmaceutical composition comprises:

[a] a compound of Formula I chosen from:
N-(3-Amino-propyl)-N-{1-[3-(3-cyano-benzyl)-7-hydroxy-4-oxo-4H-chromen-2-yl]-2-methyl-propyl}-3-fluoro-4-methyl-benzamide;
4-Acetyl-N-(3-amino-propyl)-N-[1-(3-benzyl-7-methoxy-4-oxo-4H-chromen-2-yl)-2-methyl-propyl]-benzamide;
2-{1-[4-(2-Amino-ethyl)-2-p-tolyl-imidazol-1-yl]-2-methyl-propyl}-3-benzyl-4-oxo-4H-chromene-7-carbonitrile;
3-Benzyl-2-[1-(4,4-dimethyl-2-p-tolyl-4,5-dihydro-imidazol-1-yl)-2-methyl-propyl]-4-oxo-4H-chromene-7-carbonitrile;
Benzo[1,3]dioxole-5-carboxylic acid (3-amino-propyl)-{1-[7-chloro-3-(3-cyano-benzyl)-4-oxo-4H-chromen-2-yl]-2-methyl-propyl}-amide;
N-(3-Amino-propyl)-N-{1-[7-chloro-3-(3-cyano-benzyl)-4-oxo-4H-chromen-2-yl]-2-methyl-propyl}-4-methyl-benzamide;
N-(3-Amino-propyl)-N-[1-(7-chloro-3-naphthalen-1-ylmethyl-4-oxo-4H-chromen-2-yl)-2-methyl-propyl]-2-methoxy-acetamide;
4-Acetyl-N-(3-amino-propyl)-N-[1-(3-benzyl-7-cyano-4-oxo-4H-chromen-2-yl)-2-methyl-propyl]-benzamide;
N-(3-Amino-propyl)-N-{1-[7-chloro-3-(3-cyano-benzyl)-4-oxo-4H-chromen-2-yl]-2-methyl-propyl}-3-fluoro-4-methyl-benzamide;
Benzo[1,3]dioxole-5-carboxylic acid (3-amino-propyl)-[1-(3-benzyl-7-cyano-4-oxo-4H-chromen-2-yl)-2-methyl-propyl]-amide;
Benzo[1,3]dioxole-5-carboxylic acid (3-amino-propyl)-[1-(3-benzyl-7-fluoro-4-oxo-4H-chromen-2-yl)-2-methyl-propyl]-amide;
3-Benzyl-2-[1-(4,4-dimethyl-2-p-tolyl-4,5-dihydro-imidazol-1-yl)-2-methyl-propyl]-7-hydroxy-chromen-4-one;
N-(3-Amino-propyl)-N-[1-(3-benzyl-7-fluoro-4-oxo-4H-chromen-2-yl)-2-methyl-propyl]-3-fluoro-4-methyl-benzamide;
N-(3-Amino-propyl)-N-[1-(3-benzyl-7-methoxy-4-oxo-4H-chromen-2-yl)-2-methyl-propyl]-3-fluoro-4-methyl-benzamide;
N-(3-Amino-propyl)-N-[1-(3-benzyl-7-methoxy-4-oxo-4H-chromen-2-yl)-2-methyl-propyl]-4-methyl-benzamide;
3-Benzyl-2-[1-(4,4-dimethyl-2-p-tolyl-4,5-dihydro-imidazol-1-yl)-2-methyl-propyl]-7-methoxy-chromen-4-one;
3-Benzyl-7-fluoro-2-{1-[2-(3-fluoro-4-methyl-phenyl)-4,4-dimethyl-4,5-dihydro-imidazol-1-yl]-2-methyl-propyl}-chromen-4-one;
3-Benzyl-2-[1-(4,4-dimethyl-2-p-tolyl-4,5-dihydro-imidazol-1-yl)-2-methyl-propyl]-7-fluoro-chromen-4-one;
3-Benzyl-2-{1-[2-(3-fluoro-4-methyl-phenyl)-4,4-dimethyl-4,5-dihydro-imidazol-1-yl]-2-methyl-propyl}-7-cyano-chromen-4-one;
N-(3-Amino-propyl)-N-[1-(3-benzyl-7-fluoro-4-oxo-4H-chromen-2-yl)-2-methyl-propyl]-4-methyl-benzamide;
4-Acetyl-N-(3-amino-propyl)-N-[1-(3-benzyl-7-chloro-4-oxo-4H-chromen-2-yl)-2-methyl-propyl]-benzamide;
N-(3-Amino-propyl)-3-fluoro-N-{1-[7-fluoro-3-(3-methoxy-benzyl)-4-oxo-4H-chromen-2-yl]-2-methyl-propyl}-4-methyl-benzamide;
4-Acetyl-N-(3-amino-propyl)-N-{1-[7-chloro-3-(3-cyano-benzyl)-4-oxo-4H-chromen-2-yl]-2-methyl-propyl}-benzamide;
(2-{1-[1-(3-Benzyl-7-chloro-4-oxo-4H-chromen-2-yl)-2-methyl-propyl]-2-p-tolyl-1H-imidazol-4-yl}-ethyl)-carbamic acid benzyl ester;
2-[1-(2-Benzo[1,3]dioxol-5-yl-4,4-dimethyl-4,5-dihydro-imidazol-1-yl)-2-methyl-propyl]-3-benzyl-7-cyano-chromen-4-one;
4-Acetyl-N-(3-amino-propyl)-N-[1-(3-benzyl-7-fluoro-4-oxo-4H-chromen-2-yl)-2-methyl-propyl]-benzamide;
N-(3-Amino-propyl)-N-[1-(3-benzyl-7-chloro-4-oxo-4H-chromen-2-yl)-2-methyl-propyl]-3-fluoro-4-methyl-benzamide;
N-(3-Amino-propyl)-N-{1-[7-fluoro-3-(3-methoxy-benzyl)-4-oxo-4H-chromen-2-yl]-2-methyl-propyl}-4-methoxy-benzamide;
Benzo[1,3]dioxole-5-carboxylic acid (3-amino-propyl)-{1-[7-fluoro-3-(3-methoxy-benzyl)-4-oxo-4H-chromen-2-yl]-2-methyl-propyl}-amide;
2-{1-[4-(2-Amino-ethyl)-2-p-tolyl-imidazol-1-yl]-2-methyl-propyl}-3-benzyl-7-chloro-chromen-4-one;
N-(3-Amino-propyl)-N-[1-(3-benzyl-7-hydroxy-4-oxo-4H-chromen-2-yl)-2-methyl-propyl]-3-fluoro-4-methyl-benzamide;
Benzo[1,3]dioxole-5-carboxylic acid (3-amino-propyl)-[1-(3-benzyl-7-chloro-4-oxo-4H-chromen-2-yl)-2-methyl-propyl]-amide;
N-(3-Amino-propyl)-N-[1-(3-benzyl-7-hydroxy-4-oxo-4H-chromen-2-yl)-2-methyl-propyl]-4-methyl-benzamide;
N-(3-Amino-propyl)-N-{1-[7-fluoro-3-(3-methoxy-benzyl)-4-oxo-4H-chromen-2-yl]-2-methyl-propyl}-2-methoxy-acetamide;
N-(3-Amino-propyl)-N-{1-[7-fluoro-3-(3-methoxy-benzyl)-4-oxo-4H-chromen-2-yl]-2-methyl-propyl}-4-methyl-benzamide;
N-(3-Amino-propyl)-N-[(3-benzyl-7-chloro-4-oxo-4H-chromen-2-yl)-cyclopropyl-methyl]-4-methyl-benzamide;
2,3-Dihydro-benzo[1,4]dioxine-6-carboxylic acid (3-amino-propyl)-[1-(3-benzyl-7-chloro-4-oxo-4H-chromen-2-yl)-2-methyl-propyl]-amide;
N-(3-Amino-propyl)-3-fluoro-N-{1-[7-cyano-3-(3-methoxy-benzyl)-4-oxo-4H-chromen-2-yl]-2-methyl-propyl}-4-methyl-benzamide;
3-Benzyl-7-chloro-2-[2-methyl-1-(2-p-tolyl-4,5-dihydro-imidazol-1-yl)-propyl]-chromen-4-one;
3-Benzyl-7-fluoro-2-{1-[2-(3-fluoro-4-methyl-phenyl)-4,5-dihydro-imidazol-1-yl]-2-methyl-propyl}-chromen-4-one;
2-[1-(4-Aminomethyl-2-p-tolyl-imidazol-1-yl)-2-methyl-propyl]-3-benzyl-7-chloro-chromen-4-one;
3-Benzyl-7-methoxy-2-[2-methyl-1-(2-p-tolyl-4,5-dihydro-imidazol-1-yl)-propyl]-chromen-4-one;
2-[1-(2-Benzo[1,3]dioxol-5-yl-4,5-dihydro-imidazol-1-yl)-2-methyl-propyl]-3-benzyl-7-chloro-chromen-4-one;
4-Acetyl-N-(3-amino-propyl)-N-[1-(3-benzyl-7-chloro-4-oxo-4H-chromen-2-yl)-2-methyl-propyl]-benzamide;
4-Acetyl-N-(3-amino-propyl)-N-{1-[7-cyano-3-(3-methoxy-benzyl)-4-oxo-4H-chromen-2-yl]-2-methyl-propyl}-benzamide;
3-Benzyl-7-chloro-2-{1-[2-(3-fluoro-4-methyl-phenyl)-4,5-dihydro-imidazol-1-yl]-2-methyl-propyl}-chromen-4-one;
3-Benzyl-7-fluoro-2-[2-methyl-1-(2-p-tolyl-4,5-dihydro-imidazol-1-yl)-propyl]-chromen-4-one;
2-[1-(2-Benzo[1,3]dioxol-5-yl-4,5-dihydro-imidazol-1-yl)-2-methyl-propyl]-3-benzyl-7-fluoro-chromen-4-one;
N-(3-Amino-propyl)-N-{1-[7-cyano-3-(3-methoxy-benzyl)-4-oxo-4H-chromen-2-yl]-2-methyl-propyl}-4-methyl-benzamide;
Benzo[1,3]dioxole-5-carboxylic acid (3-amino-propyl)-{1-[7-cyano-3-(3-methoxy-benzyl)-4-oxo-4H-chromen-2-yl]-2-methyl-propyl}-amide;
3-(2-{1-[2-(3-Fluoro-4-methyl-phenyl)-4,5-dihydro-imidazol-1-yl]-2-methyl-propyl}-7-methoxy-4-oxo-4H-chromen-3-ylmethyl)-benzonitrile;
3-{7-Chloro-2-[2-methyl-1-(2-p-tolyl-4,5-dihydro-imidazol-1-yl)-propyl]-4-oxo-4H-chromen-3-ylmethyl}-benzonitrile;
3-{7-Methoxy-2-[2-methyl-1-(2-p-tolyl-4,5-dihydro-imidazol-1-yl)-propyl]-4-oxo-4H-chromen-3-ylmethyl}-benzonitrile;
3-{7-Fluoro-2-[2-methyl-1-(2-p-tolyl-4,5-dihydro-imidazol-1-yl)-propyl]-4-oxo-4H-chromen-3-ylmethyl}-benzonitrile;
N-(3-Amino-propyl)-N-{1-[7-chloro-3-(3-methoxy-benzyl)-4-oxo-4H-chromen-2-yl]-2-methyl-propyl}-3-fluoro-4-methyl-benzamide;
N-(3-Amino-propyl)-N-{1-[3-(3-cyano-benzyl)-7-methoxy-4-oxo-4H-chromen-2-yl]-2-methyl-propyl}-4-methyl-benzamide;
N-(3-Amino-propyl)-N-{1-[3-(3-cyano-benzyl)-7-methoxy-4-oxo-4H-chromen-2-yl]-2-methyl-propyl}-3-fluoro-4-methyl-benzamide;
3-{2-[1-(2-Benzo[1,3]dioxol-5-yl-4,5-dihydro-imidazol-1-yl)-2-methyl-propyl]-7-fluoro-4-oxo-4H-chromen-3-ylmethyl}-benzonitrile;
3-Benzyl-2-{1-[2-(3-fluoro-4-methyl-phenyl)-4,5-dihydro-imidazol-1-yl]-2-methyl-propyl}-4-oxo-4H-chromene-7-carbonitrile;
2-[1-(2-Benzo[1,3]dioxol-5-yl-4,5-dihydro-imidazol-1-yl)-2-methyl-propyl]-3-benzyl-4-oxo-4H-chromene-7-carbonitrile;
3-(7-Chloro-2-{1-[2-(3-fluoro-4-methyl-phenyl)-4,5-dihydro-imidazol-1-yl]-2-methyl-propyl}-4-oxo-4H-chromen-3-ylmethyl)-benzonitrile;
3-{2-[1-(2-Benzo[1,3]dioxol-5-yl-4,5-dihydro-imidazol-1-yl)-2-methyl-propyl]-7-methoxy-4-oxo-4H-chromen-3-ylmethyl}-benzonitrile;
3-(7-Fluoro-2-{1-[2-(3-fluoro-4-methyl-phenyl)-4,5-dihydro-imidazol-1-yl]-2-methyl-propyl}-4-oxo-4H-chromen-3-ylmethyl)-benzonitrile;
Benzo[1,3]dioxole-5-carboxylic acid (3-amino-propyl)-{1-[7-cyano-3-(3-cyano-benzyl)-4-oxo-4H-chromen-2-yl]-2-methyl-propyl}-amide;
Benzo[1,3]dioxole-5-carboxylic acid (3-amino-propyl)-{1-[3-(3-cyano-benzyl)-7-methoxy-4-oxo-4H-chromen-2-yl]-2-methyl-propyl}-amide;
Benzo[1,3]dioxole-5-carboxylic acid (3-amino-propyl)-{1-[3-(3-cyano-benzyl)-7-fluoro-4-oxo-4H-chromen-2-yl]-2-methyl-propyl}-amide;
N-(3-Amino-propyl)-N-{1-[3-(3-cyano-benzyl)-7-fluoro-4-oxo-4H-chromen-2-yl]-2-methyl-propyl}-4-methyl-benzamide;
Benzo[1,3]dioxole-5-carboxylic acid (3-amino-propyl)-{1-[7-chloro-3-(3-methoxy-benzyl)-4-oxo-4H-chromen-2-yl]-2-methyl-propyl}-amide;
N-(3-Amino-propyl)-N-{1-[7-cyano-3-(3-cyano-benzyl)-4-oxo-4H-chromen-2-yl]-2-methyl-propyl}-4-methyl-benzamide;
3-Benzyl-2-[2-methyl-1-(2-p-tolyl-4,5-dihydro-imidazol-1-yl)-propyl]-4-oxo-4H-chromene-7-carbonitrile;
2-[1-(2-Benzo[1,3]dioxol-5-yl-4,5-dihydro-imidazol-1-yl)-2-methyl-propyl]-3-benzyl-7-methoxy-chromen-4-one;
N-(3-Amino-propyl)-N-{1-[7-chloro-3-(3-methoxy-benzyl)-4-oxo-4H-chromen-2-yl]-2-methyl-propyl}-2-methoxy-acetamide;
N-(3-Amino-propyl)-N-{1-[7-cyano-3-(3-cyano-benzyl)-4-oxo-4H-chromen-2-yl]-2-methyl-propyl}-3-fluoro-4-methyl-benzamide;
N-(3-Amino-propyl)-N-{1-[3-(3-cyano-benzyl)-7-fluoro-4-oxo-4H-chromen-2-yl]-2-methyl-propyl}-3-fluoro-4-methyl-benzamide;
2-{1-[4-(2-Amino-ethyl)-2-p-tolyl-imidazol-1-yl]-2-methyl-propyl}-7-chloro-3-(3-methoxy-benzyl)-chromen-4-one;
2-{1-[4-(2-Amino-ethyl)-2-(3-fluoro-4-methyl-phenyl)-imidazol-1-yl]-2-methyl-propyl}-3-benzyl-7-fluoro-chromen-4-one;
2-{1-[4-(2-Amino-ethyl)-2-(3-fluoro-4-methyl-phenyl)-imidazol-1-yl]-2-methyl-propyl}-3-benzyl-4-oxo-4H-chromene-7-carbonitrile;
2-{1-[4-(2-Amino-ethyl)-2-p-tolyl-imidazol-1-yl]-2-methyl-propyl}-3-benzyl-7-fluoro-chromen-4-one;
2-{1-[4-(2-Amino-ethyl)-2-(3-fluoro-4-methyl-phenyl)-imidazol-1-yl]-2-methyl-propyl}-7-chloro-3-(3-methoxy-benzyl)-chromen-4-one;
2-{1-[4-(2-Amino-ethyl)-2-(3-fluoro-4-methyl-phenyl)-imidazol-1-yl]-2-methyl-propyl}-3-benzyl-7-chloro-chromen-4-one;
3-Benzyl-2-(1-[2-(3-fluoro-4-methyl-phenyl)-4,5-dihydro-imidazol-1-yl]-2-methyl-propyl)-7-methoxy-chromen-4-one;
3-(2-{1-[4-(2-Amino-ethyl)-2-p-tolyl-imidazol-1-yl]-2-methyl-propyl}-7-chloro-4-oxo-4H-chromen-3-ylmethyl)-benzonitrile;
3-(2-{1-[4-(2-Amino-ethyl)-2-(3-fluoro-4-methyl-phenyl)-imidazol-1-yl]-2-methyl-propyl}-7-chloro-4-oxo-4H-chromen-3-ylmethyl)-benzonitrile;
2-{1-[4-(2-Amino-ethyl)-2-p-tolyl-imidazol-1-yl]-2-methyl-propyl}-7-fluoro-3-(3-methoxy-benzyl)-chromen-4-one;
2-{1-[4-(2-Amino-ethyl)-2-(3-fluoro-4-methyl-phenyl)-imidazol-1-yl]-2-methyl-propyl}-3-(3-cyano-benzyl)-4-oxo-4H-chromene-7-carbonitrile;
N-(3-Amino-propyl)-3-fluoro-N-{1-[7-hydroxy-3-(3-methoxy-benzyl)-4-oxo-4H-chromen-2-yl]-2-methyl-propyl}-4-methyl-benzamide;
2-{1-[4-(2-Amino-ethyl)-2-p-tolyl-imidazol-1-yl]-2-methyl-propyl}-3-(3-methoxy-benzyl)-4-oxo-4H-chromene-7-carbonitrile;
N-(3-Amino-propyl)-N-[1-(3-benzyl-7-cyano-4-oxo-4H-chromen-2-yl)-2-methyl-propyl]-4-ethoxy-benzamide;
N-(3-Amino-propyl)-N-[1-(3-benzyl-7-cyano-4-oxo-4H-chromen-2-yl)-2-methyl-propyl]-6-trifluoromethyl-nicotinamide;
N-(3-Amino-propyl)-N-[1-(3-benzyl-7-fluoro-4-oxo-4H-chromen-2-yl)-2-methyl-propyl]-6-trifluoromethyl-nicotinamide;
N-(3-Amino-propyl)-N-[1-(3-benzyl-7-fluoro-4-oxo-4H-chromen-2-yl)-2-methyl-propyl]-isonicotinamide;
N-(3-Amino-propyl)-N-[1-(3-benzyl-7-chloro-4-oxo-4H-chromen-2-yl)-2-methyl-propyl]-4-cyano-benzamide;
4-Acetylamino-N-(3-amino-propyl)-N-[1-(3-benzyl-7-chloro-4-oxo-4H-chromen-2-yl)-2-methyl-propyl]-benzamide;
N-(3-Amino-propyl)-N-[1-(3-benzyl-7-chloro-4-oxo-4H-chromen-2-yl)-2-methyl-propyl]-6-trifluoromethyl-nicotinamide;
Benzo[1,2,3]thiadiazole-5-carboxylic acid (3-amino-propyl)-[1-(3-benzyl-7-chloro-4-oxo-4H-chromen-2-yl)-2-methyl-propyl]-amide;
Benzo[1,2,3]thiadiazole-5-carboxylic acid (3-amino-propyl)-[1-(3-benzyl-7-fluoro-4-oxo-4H-chromen-2-yl)-2-methyl-propyl]-amide;
4-Acetylamino-N-(3-amino-propyl)-N-[1-(3-benzyl-7-fluoro-4-oxo-4H-chromen-2-yl)-2-methyl-propyl]-benzamide;
Benzo[1,2,3]thiadiazole-5-carboxylic acid (3-amino-propyl)-[1-(3-benzyl-7-fluoro-4-oxo-4H-chromen-2-yl)-2-methyl-propyl]-amide;
N-(3-Amino-propyl)-N-[1-(3-benzyl-7-chloro-4-oxo-4H-chromen-2-yl)-2-methyl-propyl]-nicotinamide;
N-(3-Amino-propyl)-N-[1-(3-benzyl-7-fluoro-4-oxo-4H-chromen-2-yl)-2-methyl-propyl]-4-methoxy-benzamide;
Benzo[1,2,3]thiadiazole-5-carboxylic acid (3-amino-propyl)-[1-(3-benzyl-7-cyano-4-oxo-4H-chromen-2-yl)-2-methyl-propyl]-amide;
5-Methyl-pyrazine-2-carboxylic acid (3-amino-propyl)-[1-(3-benzyl-7-chloro-4-oxo-4H-chromen-2-yl)-2-methyl-propyl]-amide;
N-(3-Amino-propyl)-N-[1-(3-benzyl-7-cyano-4-oxo-4H-chromen-2-yl)-2-methyl-propyl]-3-dimethylamino-benzamide;
2-[1-(2-Benzo[1,3]dioxol-5-yl-4,5-dihydro-imidazol-1-yl)-2-methyl-propyl]-3-(3-methoxy-benzyl)-4-oxo-4H-chromene-7-carbonitrile;
7-Chloro-3-(3-methoxy-benzyl)-2-[2-methyl-1-(2-p-tolyl-4,5-dihydro-imidazol-1-yl)-propyl]-chromen-4-one;
7-Chloro-2-{1-[2-(3-fluoro-4-methyl-phenyl)-4,5-dihydro-imidazol-1-yl]-2-methyl-propyl}-3-(3-methoxy-benzyl)-chromen-4-one;
2-[1-(2-Benzo[1,3]dioxol-5-yl-4,5-dihydro-imidazol-1-yl)-2-methyl-propyl]-7-chloro-3-(3-methoxy-benzyl)-chromen-4-one;
7-Fluoro-3-(3-methoxy-benzyl)-2-[2-methyl-1-(2-p-tolyl-4,5-dihydro-imidazol-1-yl)-propyl]-chromen-4-one;
7-Fluoro-2-{1-[2-(3-fluoro-4-methyl-phenyl)-4,5-dihydro-imidazol-1-yl]-2-methyl-propyl}-3-(3-methoxy-benzyl)-chromen-4-one;
2-{1-[4-(2-Amino-ethyl)-2-(3-fluoro-4-methyl-phenyl)-imidazol-1-yl]-2-methyl-propyl}-7-fluoro-3-(3-methoxy-benzyl)-chromen-4-one;
2-{1-[4-(2-Acetylamino-ethyl)-2-(3-fluoro-4-methyl-phenyl)-imidazol-1-yl]-2-methyl-propyl}-3-(3-methoxy-benzyl)-4-oxo-4H-chromene-7-carboxylic acid amide;
3-(2-{1-[4-(2-Amino-ethyl)-2-(3-fluoro-4-methyl-phenyl)-imidazol-1-yl]-2-methyl-propyl}-7-fluoro-4-oxo-4H-chromen-3-ylmethyl)-benzonitrile;
N-{1-[1-(3-Benzyl-7-chloro-4-oxo-4H-chromen-2-yl)-2-methyl-propyl]-2-p-tolyl-1H-imidazol-4-ylmethyl}-acetamide;
Benzo[b]thiophene-2-carboxylic acid (3-amino-propyl)-[1-(3-benzyl-7-cyano-4-oxo-4H-chromen-2-yl)-2-methyl-propyl]-amide;
1-Methyl-1H-indole-2-carboxylic acid (3-amino-propyl)-[1-(3-benzyl-7-cyano-4-oxo-4H-chromen-2-yl)-2-methyl-propyl]-amide;
5-tert-Butyl-2-methyl-2H-pyrazole-3-carboxylic acid (3-amino-propyl)-[1-(3-benzyl-7-cyano-4-oxo-4H-chromen-2-yl)-2-methyl-propyl]-amide;
2,5-Dimethyl-2H-pyrazole-3-carboxylic acid (3-amino-propyl)-[1-(3-benzyl-7-cyano-4-oxo-4H-chromen-2-yl)-2-methyl-propyl]-amide;
5-tert-Butyl-2-methyl-2H-pyrazole-3-carboxylic acid (3-amino-propyl)-[1-(3-benzyl-7-fluoro-4-oxo-4H-chromen-2-yl)-2-methyl-propyl]-amide;
2,5-Dimethyl-2H-pyrazole-3-carboxylic acid (3-amino-propyl)-[1-(3-benzyl-7-chloro-4-oxo-4H-chromen-2-yl)-2-methyl-propyl]-amide;
2,5-Dimethyl-2H-pyrazole-3-carboxylic acid (3-amino-propyl)-[1-(3-benzyl-7-fluoro-4-oxo-4H-chromen-2-yl)-2-methyl-propyl]-amide;
5-Methyl-2H-pyrazole-3-carboxylic acid (3-amino-propyl)-[1-(3-benzyl-7-cyano-4-oxo-4H-chromen-2-yl)-2-methyl-propyl]-amide;
5-Methyl-2H-pyrazole-3-carboxylic acid (3-amino-propyl)-[1-(3-benzyl-7-fluoro-4-oxo-4H-chromen-2-yl)-2-methyl-propyl]-amide;
Furan-2-carboxylic acid (3-amino-propyl)-[1-(3-benzyl-7-fluoro-4-oxo-4H-chromen-2-yl)-2-methyl-propyl]-amide;
Furan-2-carboxylic acid (3-amino-propyl)-[1-(3-benzyl-7-chloro-4-oxo-4H-chromen-2-yl)-2-methyl-propyl]-amide;
2,5-Dimethyl-furan-3-carboxylic acid (3-amino-propyl)-[1-(3-benzyl-7-cyano-4-oxo-4H-chromen-2-yl)-2-methyl-propyl]-amide;
2,5-Dimethyl-furan-3-carboxylic acid (3-amino-propyl)-[1-(3-benzyl-7-fluoro-4-oxo-4H-chromen-2-yl)-2-methyl-propyl]-amide;
5-Methyl-thiophene-2-carboxylic acid (3-amino-propyl)-[1-(3-benzyl-7-cyano-4-oxo-4H-chromen-2-yl)-2-methyl-propyl]-amide;
5-Methyl-thiophene-2-carboxylic acid (3-amino-propyl)-[1-(3-benzyl-7-fluoro-4-oxo-4H-chromen-2-yl)-2-methyl-propyl]-amide;
5-Methyl-isoxazole-3-carboxylic acid (3-amino-propyl)-[1-(3-benzyl-7-chloro-4-oxo-4H-chromen-2-yl)-2-methyl-propyl]-amide;
5-Methyl-2-trifluoromethyl-furan-3-carboxylic acid (3-amino-propyl)-[1-(3-benzyl-7-cyano-4-oxo-4H-chromen-2-yl)-2-methyl-propyl]-amide;
5-Methyl-isoxazole-3-carboxylic acid (3-amino-propyl)-[1-(3-benzyl-7-fluoro-4-oxo-4H-chromen-2-yl)-2-methyl-propyl]-amide;
5-Methyl-isoxazole-3-carboxylic acid (3-amino-propyl)-[1-(3-benzyl-7-cyano-4-oxo-4H-chromen-2-yl)-2-methyl-propyl]-amide;
Benzo[c]isoxazole-3-carboxylic acid (3-amino-propyl)-[1-(3-benzyl-7-chloro-4-oxo-4H-chromen-2-yl)-2-methyl-propyl]-amide;
Benzo[c]isoxazole-3-carboxylic acid (3-amino-propyl)-[1-(3-benzyl-7-fluoro-4-oxo-4H-chromen-2-yl)-2-methyl-propyl]-amide;
1-Methyl-1H-pyrrole-2-carboxylic acid (3-amino-propyl)-[1-(3-benzyl-7-fluoro-4-oxo-4H-chromen-2-yl)-2-methyl-propyl]-amide;
1-Methyl-1H-imidazole-4-carboxylic acid (3-amino-propyl)-[(R)-1-(3-benzyl-7-cyano-4-oxo-4H-chromen-2-yl)-2-methyl-propyl]-amide;
N-(3-Amino-propyl)-N—[(R)-1-(3-benzyl-7-cyano-4-oxo-4H-chromen-2-yl)-2-methyl-propyl]-3-dimethylamino-benzamide;
5-Methyl-2-trifluoromethyl-furan-3-carboxylic acid (3-amino-propyl)-[(R)-1-(3-benzyl-7-cyano-4-oxo-4H-chromen-2-yl)-2-methyl-propyl]-amide;
5-Methyl-isoxazole-3-carboxylic acid (3-amino-propyl)-[(R)-1-(3-benzyl-7-cyano-4-oxo-4H-chromen-2-yl)-2-methyl-propyl]-amide;
1-Methyl-1H-imidazole-4-carboxylic acid (3-amino-propyl)-[(R)-1-(3-benzyl-7-fluoro-4-oxo-4H-chromen-2-yl)-2-methyl-propyl]-amide;
1-Methyl-1H-pyrrole-2-carboxylic acid (3-amino-propyl)-[(R)-1-(3-benzyl-7-fluoro-4-oxo-4H-chromen-2-yl)-2-methyl-propyl]-amide;
Benzo[c]isoxazole-3-carboxylic acid (3-amino-propyl)-[(R)-1-(3-benzyl-7-fluoro-4-oxo-4H-chromen-2-yl)-2-methyl-propyl]-amide; and
5-Methyl-isoxazole-3-carboxylic acid (3-amino-propyl)-[(R)-1-(3-benzyl-7-fluoro-4-oxo-4H-chromen-2-yl)-2-methyl-propyl]-amide, and

pharmaceutically acceptable salts thereof,

[b] a chemotherapeutic agent selected from doxorubucin, cisplatin, 5-fluoruracil, gemcitabine, irinotecan, docetaxel, capecitabine and carboplatin; and optionally

[c] a pharmaceutically acceptable excipient.

In one embodiment, in the pharmaceutical composition, the pharmaceutically acceptable salt of a compound of Formula (I) is a hydrochloride salt.

In yet another embodiment, the pharmaceutical composition comprises N-(3-Aminopropyl)-N—[(R)-1-3-benzyl-7-chloro-4-oxo-4H-chromen-2-yl-2-methyl-propyl]-4-methyl-benzamide or a pharmaceutically acceptable salt thereof (e.g., the hydrochloride salt) in combination with G-CSF.

In yet another embodiment, the pharmaceutical composition comprises N-(3-Aminopropyl)-N—[(R)-1-3-benzyl-7-chloro-4-oxo-4H-chromen-2-yl-2-methyl-propyl]-4-methyl-benzamide or a pharmaceutically acceptable salt thereof (e.g., the hydrochloride salt) in combination with doxorubicin.

In another embodiment, the pharmaceutical composition comprises N-(3-Aminopropyl)-N—[(R)-1-3-benzyl-7-chloro-4-oxo-4H-chromen-2-yl-2-methyl-propyl]-4-methyl-benzamide or a pharmaceutically acceptable salt thereof (e.g., the hydrochloride salt) in combination with cisplatin.

In another embodiment, the pharmaceutical composition comprises N-(3-Aminopropyl)-N—[(R)-1-3-benzyl-7-chloro-4-oxo-4H-chromen-2-yl-2-methyl-propyl]-4-methyl-benzamide or a pharmaceutically acceptable salt thereof (e.g., the hydrochloride salt) in combination with gemcitabine.

In another embodiment, the pharmaceutical composition comprises N-(3-Aminopropyl)-N—[(R)-1-3-benzyl-7-chloro-4-oxo-4H-chromen-2-yl-2-methyl-propyl]-4-methyl-benzamide or a pharmaceutically acceptable salt thereof (e.g., the hydrochloride salt) in combination with irinotecan.

In another embodiment, the pharmaceutical composition comprises N-(3-Aminopropyl)-N—[(R)-1-3-benzyl-7-chloro-4-oxo-4H-chromen-2-yl-2-methyl-propyl]-4-methyl-benzamide or a pharmaceutically acceptable salt thereof (e.g., the hydrochloride salt) in combination with carboplatin

In another embodiment, the pharmaceutical composition comprises N-(3-Aminopropyl)-N—[(R)-1-3-benzyl-7-chloro-4-oxo-4H-chromen-2-yl-2-methyl-propyl]-4-methyl-benzamide or a pharmaceutically acceptable salt thereof (e.g., the hydrochloride salt) in combination with docetaxel.

In another embodiment, the pharmaceutical composition comprises N-(3-Aminopropyl)-N—[(R)-1-3-benzyl-7-chloro-4-oxo-4H-chromen-2-yl-2-methyl-propyl]-4-methyl-benzamide or a pharmaceutically acceptable salt thereof (e.g., the hydrochloride salt) in combination with capecitabine.

The compounds, pharmaceutical compositions, and/or methods of using such compounds or compositions may find use in a variety of biological applications.

For example, the present invention relates to the development of inhibitors and modulators of mitotic kinesins, in particular KSP, for the treatment of disorders associated with cell proliferation. In another aspect, the present invention relates to the the development of inhibitors and modulators of mitotic kinesins, in particular KSP, in combination with other chemotherapeutic agents for the treatment of disorders associated with cell proliferation.

In accordance with the present invention, specific inhibition of cellular proliferation, e.g., by the chromenone derivative, is accomplished by inhibiting or modulating mitotic kinesins, but not other kinesins (e.g., transport kinesins). Thus, the present invention capitalizes on the finding that perturbation of mitotic kinesin function causes malformation or dysfunction of mitotic spindles, frequently resulting in cell cycle arrest and cell death.

As will be appreciated by those skilled in the art, mitosis may be altered in a variety of ways; that is, one can affect mitosis either by increasing or decreasing the activity of a component in the mitotic pathway. Stated differently, mitosis may be affected (e.g., disrupted) by disturbing equilibrium, either by inhibiting or activating certain components. Similar approaches may be used to alter meiosis.

In one embodiment, the chromenone derivative, or compositions and methods of the present invention comprising the chromenone derivative are used to modulate mitotic spindle formation, thus causing prolonged cell cycle arrest in mitosis.

By “modulate” herein is meant altering mitotic spindle formation, including increasing and decreasing spindle formation.

By “mitotic spindle formation” herein is meant organization of microtubules into bipolar structures by mitotic kinesins.

By “mitotic spindle dysfunction” herein is meant mitotic arrest and monopolar spindle formation.

The compounds and/or compositions of the invention are useful to bind to and/or modulate the activity of mitotic kinesin, KSP.

In one embodiment, the KSP is human KSP, although KSP kinesins from other organisms may also be used. In this context, modulate means either increasing or decreasing spindle pole separation, causing malformation, i.e., splaying, of mitotic spindle poles, or otherwise causing morphological perturbation of the mitotic spindle.

Also included within the definition of KSP for these purposes are variants and/or fragments of KSP. See for example, U.S. patent application “Methods of Screening for Modulators of Cell Proliferation and Methods of Diagnosing Cell Proliferation States”, filed Oct. 27, 1999 (U.S. Ser. No. 09/428,156), issued as U.S. Pat. No. 6,617,115, hereby incorporated by reference in its entirety.

In addition, other mitotic kinesins may be used in the present invention. However, the compositions of the invention have been shown to have specificity for KSP.

Assays or screening methods to show various KSP kinesin activities by chromenone compounds and/or pharmaceutical compositions thereof are described in U.S. Pat. No. 6,924,376, which is hereby incorporated by reference in its entirety. For example, kinesin activities identified in the art, include the ability to affect ATP hydrolysis; microtubule binding; gliding and polymerization/depolymerization (effects on microtubule dynamics); binding to other proteins of the spindle; binding to proteins involved in cell-cycle control; serving as a substrate to other enzymes; such as kinases or proteases; and specific kinesin cellular activities such as spindle pole separation.

Disease states which can be treated by compounds, compositions, and/or methods of the present invention may include, but are not limited to, cancer, autoimmune disease, arthritis, graft rejection, inflammatory bowel disease, proliferation induced after medical procedures, including, but not limited to, surgery, angioplasty, and the like. It is appreciated that in some cases the cells may not be in a hyper or hypo proliferation state (abnormal state) and still require treatment. For example, during wound healing, the cells may be proliferating “normally”, but proliferation enhancement may be desired.

In general, compounds, pharmaceutical compositions and/or methods of the present invention may differ in their selectivity and are used to treat diseases of proliferating cells, which generally may include, but not limited to cancer, hyperplasias, restenosis, cardiac hypertrophy, immune disorders, inflammation and the like.

Specific cancers types, which may be treated by compounds, compositions and methods of the invention may include, but are not limited to:

Cardiac: sarcoma (e.g., such as angiosarcoma, fibrosarcoma, rhabdomyosarcoma, liposarcoma and the like), myxoma, rhabdomyoma, fibroma, lipoma and teratoma;

Lung: bronchogenic carcinoma (e.g., such as squamous cell, undifferentiated small cell, undifferentiated large cell, adenocarcinoma and the like), alveolar (e.g., such as bronchiolar) carcinoma, bronchial adenoma, sarcoma, lymphoma, chondromatous hamartoma, mesothelioma;

Gastrointestinal: esophagus (e.g., such as squamous cell carcinoma, adenocarcinoma, leiomyosarcoma, lymphoma and the like), stomach (e.g., such as carcinoma, lymphoma, leiomyosarcoma and the like), pancreas (e.g., such as ductal adenocarcinoma, insulinoma, glucagonoma, gastrinoma, carcinoid tumors, vipoma and the like), small bowel (e.g., such as adenocarcinoma, lymphoma, carcinoid tumors, Karposi's sarcoma, leiomyoma, hemangioma, lipoma, neurofibroma, fibroma, and the like), large bowel (e.g., such as adenocarcinoma, tubular adenoma, villous adenoma, hamartoma, leiomyoma and the like);

Genitourinary tract: kidney (e.g., such as adenocarcinoma, Wilm's tumor [nephroblastoma], lymphoma, leukemia, and the like), bladder and urethra (e.g., such as squamous cell carcinoma, transitional cell carcinoma, adenocarcinoma and the), prostate (e.g., such as adenocarcinoma, sarcoma), testis (e.g., such as seminoma, teratoma, embryonal carcinoma, teratocarcinoma, choriocarcinoma, sarcoma, interstitial cell carcinoma, fibroma, fibroadenoma, adenomatoid tumors, lipoma and the like);

Liver: hepatoma (e.g., hepatocellular carcinoma and the like), cholangiocarcinoma, hepatoblastoma, angiosarcoma, hepatocellular adenoma, hemangioma;

Bone: osteogenic sarcoma (e.g., such as osteosarcoma and the like), fibrosarcoma, malignant fibrous histiocytoma, chondrosarcoma, Ewing's sarcoma, malignant lymphoma (e.g., such as reticulum cell sarcoma), multiple myeloma, malignant giant cell tumor chordoma, osteochronfroma (e.g., such as osteocartilaginous exostoses), benign chondroma, chondroblastoma, chondromyxofibroma, osteoid osteoma and giant cell tumors;

Nervous system: skull (e.g., such as osteoma, hemangioma, granuloma, xanthoma, osteitis deformans and the like), meninges (e.g., such as meningioma, meningiosarcoma, gliomatosis and the like), brain (e.g., such as astrocytoma, medulloblastoma, glioma, ependymoma, germinoma [pinealoma], glioblastoma multiform, oligodendroglioma, schwannoma, retinoblastoma, congenital tumors and the like), spinal cord (e.g., such as neurofibroma, meningioma, glioma, sarcoma and the like);

Gynecological: uterus (e.g., such as endometrial carcinoma and the like), cervix (e.g., such as cervical carcinoma, pre-tumor cervical dysplasia and the like), ovaries (e.g., such as ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma, and the like), vulva (e.g., such as squamous cell carcinoma, intraepithelial carcinoma, adenocarcinoma, fibrosarcoma, melanoma and the like), vagina (e.g., such as clear cell carcinoma, squamous cell carcinoma, botryoid sarcoma (embryonal rhabdomyosarcoma], fallopian tubes (carcinoma) and the like);

Hematologic: blood (e.g., such as myeloid leukemia [acute and chronic], acute lymphoblastic leukemia, chronic lymphocytic leukemia, myeloproliferative diseases, multiple myeloma, myelodysplastic syndrome and the like), Hodgkin's disease, non-Hodgkin's lymphoma [malignant lymphoma];

Skin (e.g., such as malignant melanoma, basal cell carcinoma, squamous cell carcinoma, Karposi's sarcoma, moles dysplastic nevi, lipoma, angioma, dermatofibroma, keloids, psoriasis and the like); and

Adrenal glands: neuroblastoma.

Compounds, compositions and/or methods provided herein may be useful for the treatment of solid tumor cancers, which may include solid cancer tumors associated with skin, breast, brain, cervical carcinomas, testicular carcinomas, etc.

In accordance with the present invention, the term “cancerous cell” includes a cell afflicted by any one of the above identified disease states or conditions.

In light of the foregoing, the present invention also relates to combination therapy methods for treatment of cellular proliferative diseases in a mammal in need thereof, which comprises administration of:

[a] a chromenone derivative such as defined herein, including but not limited to each express embodiment (optionally in the form of a pharmaceutical composition, e.g., further comprising a pharmaceutically acceptable excipient); in combination with

[b] a chemotherapeutic agent selected from neutropenia treatment agents, alkylating agents, antimetabolites, platinating agents; topoisomerase inhibitors, tubulin agents, signalling inhibitors (e.g., kinase inhibitors), proteasome inhibitors, and other chemotherapeutic agents, such as described herein, including but not limited to each express embodiment (optionally in the form of a pharmaceutical composition, e.g., further comprising a pharmaceutically acceptable excipient).

Specific dose levels for the active agents will depend upon considerations such as those as identified above in accordance with the present invention.

When administered as a combination, the therapeutic agents can be formulated as separate compositions that are administered at the same time or sequentially at different times, or the therapeutic agents can be administered in a single composition, provided that the active agents are not incompatible with other active agents or the formulation, or otherwise undesirably combined in a single composition.

The phrase “co-therapy” (or “combination-therapy”), in defining use of a chromenone compound derivative of the present invention and another pharmaceutical agent, such as a chemotherapeutic agent as defined above, may include the following examples:

administration of each agent in a sequential manner in a regimen to provide beneficial effects of the drug combination; and/or

co-administration of the aforementioned components in a substantially simultaneous manner (e.g., as in a single capsule having a fixed ratio of these active agents or in multiple, separate capsules for each agent, etc.).

Thus, the present invention is not limited in the sequence of administration; the chromenone derivative may be administered either prior to, at the same time with or after administration of the other chemotherapeutic agent.

The chromenone compounds and other chemotherapeutic agents may further be used in conjunction with yet other chemotherapeutic agents, additional therapies, etc. known to those skilled in the art for treatment of cellular proliferative diseases as described herein.

As described above, if combination therapies or products of the present invention are formulated as a fixed dose, such combination therapies or products will be within the accepted dosage ranges such as may be determined by one skilled in the art.

The present invention thus relates to combination therapy methods for treatment of cellular proliferative diseases in a mammal in need thereof, which comprises administering:

[a] a chromenone derivative (or a pharmaceutical composition thereof), in combination with

[b] a chemotherapeutic agent selected from neutropenia treatment agents, alkylating agents, antimetabolites, platinating agents, topoisomerase inhibitors, tubulin agents, signalling inhibitors (e.g., kinase inhibitors), and other chemotherapeutic agents (or a pharmaceutical composition thereof, which may be the same composition as for the chromenone derivative).

In a particular embodiment, the present invention relates to a combination therapy method for treatment of cellular proliferative diseases in a mammal in need thereof, which comprises:

[a] administering to said mammal a compound of formula I or a pharmaceutically acceptable salt thereof, as defined herein; and

[b] a chemotherapeutic agent selected from neutropenia treatment agents, alkylating agents, antimetabolites, platinating agents, topoisomerase inhibitors, tubulin agents, and signalling inhibitors (e.g., kinase inhibitors).

In another embodiment, the present invention relates to a combination therapy method for treatment of cellular proliferative diseases in a mammal in need thereof, which comprises administering to said mammal:

[a] a compound of formula I as defined herein; and

[b] a chemotherapeutic agent selected from neutropenia treatment agents, alkylating agents, antimetabolites, platinating agents, topoisomerase inhibitors, tubulin agents, signalling inhibitors (e.g., kinase inhibitors).

In another embodiment, the present invention relates to a combination therapy method for treatment of cellular proliferative diseases in a mammal in need thereof, which comprises administering to said mammal:

[a] a compound of Formula I chosen from
N-(3-Amino-propyl)-N-{1-[3-(3-cyano-benzyl)-7-hydroxy-4-oxo-4H-chromen-2-yl]-2-methyl-propyl}-3-fluoro-4-methyl-benzamide;
4-Acetyl-N-(3-amino-propyl)-N-[1-(3-benzyl-7-methoxy-4-oxo-4H-chromen-2-yl)-2-methyl-propyl]-benzamide;
2-{1-[4-(2-Amino-ethyl)-2-p-tolyl-imidazol-1-yl]-2-methyl-propyl}-3-benzyl-4-oxo-4H-chromene-7-carbonitrile;
3-Benzyl-2-[1-(4,4-dimethyl-2-p-tolyl-4,5-dihydro-imidazol-1-yl)-2-methyl-propyl]-4-oxo-4H-chromene-7-carbonitrile;
Benzo[1,3]dioxole-5-carboxylic acid (3-amino-propyl)-{1-[7-chloro-3-(3-cyano-benzyl)-4-oxo-4H-chromen-2-yl]-2-methyl-propyl}-amide;
N-(3-Amino-propyl)-N-{1-[7-chloro-3-(3-cyano-benzyl)-4-oxo-4H-chromen-2-yl]-2-methyl-propyl}-4-methyl-benzamide;
N-(3-Amino-propyl)-N-[1-(7-chloro-3-naphthalen-1-ylmethyl-4-oxo-4H-chromen-2-yl)-2-methyl-propyl]-2-methoxy-acetamide;
4-Acetyl-N-(3-amino-propyl)-N-[1-(3-benzyl-7-cyano-4-oxo-4H-chromen-2-yl)-2-methyl-propyl]-benzamide;
N-(3-Amino-propyl)-N-{1-[7-chloro-3-(3-cyano-benzyl)-4-oxo-4H-chromen-2-yl]-2-methyl-propyl}-3-fluoro-4-methyl-benzamide;
Benzo[1,3]dioxole-5-carboxylic acid (3-amino-propyl)-[1-(3-benzyl-7-cyano-4-oxo-4H-chromen-2-yl)-2-methyl-propyl]-amide;
Benzo[1,3]dioxole-5-carboxylic acid (3-amino-propyl)-[1-(3-benzyl-7-fluoro-4-oxo-4H-chromen-2-yl)-2-methyl-propyl]-amide;
3-Benzyl-2-[1-(4,4-dimethyl-2-p-tolyl-4,5-dihydro-imidazol-1-yl)-2-methyl-propyl]-7-hydroxy-chromen-4-one;
N-(3-Amino-propyl)-N-[1-(3-benzyl-7-fluoro-4-oxo-4H-chromen-2-yl)-2-methyl-propyl]-3-fluoro-4-methyl-benzamide;
N-(3-Amino-propyl)-N-[1-(3-benzyl-7-methoxy-4-oxo-4H-chromen-2-yl)-2-methyl-propyl]-3-fluoro-4-methyl-benzamide;
N-(3-Amino-propyl)-N-[1-(3-benzyl-7-methoxy-4-oxo-4H-chromen-2-yl)-2-methyl-propyl]-4-methyl-benzamide;
3-Benzyl-2-[1-(4,4-dimethyl-2-p-tolyl-4,5-dihydro-imidazol-1-yl)-2-methyl-propyl]-7-methoxy-chromen-4-one;
3-Benzyl-7-fluoro-2-{1-[2-(3-fluoro-4-methyl-phenyl)-4,4-dimethyl-4,5-dihydro-imidazol-1-yl]-2-methyl-propyl}-chromen-4-one;
3-Benzyl-2-[1-(4,4-dimethyl-2-p-tolyl-4,5-dihydro-imidazol-1-yl)-2-methyl-propyl]-7-fluoro-chromen-4-one;
3-Benzyl-2-{1-[2-(3-fluoro-4-methyl-phenyl)-4,4-dimethyl-4,5-dihydro-imidazol-1-yl]-2-methyl-propyl}-7-cyano-chromen-4-one;
N-(3-Amino-propyl)-N-[1-(3-benzyl-7-fluoro-4-oxo-4H-chromen-2-yl)-2-methyl-propyl]-4-methyl-benzamide;
4-Acetyl-N-(3-amino-propyl)-N-[1-(3-benzyl-7-chloro-4-oxo-4H-chromen-2-yl)-2-methyl-propyl]-benzamide;
N-(3-Amino-propyl)-3-fluoro-N-{1-[7-fluoro-3-(3-methoxy-benzyl)-4-oxo-4H-chromen-2-yl]-2-methyl-propyl}-4-methyl-benzamide;
4-Acetyl-N-(3-amino-propyl)-N-{1-[7-chloro-3-(3-cyano-benzyl)-4-oxo-4H-chromen-2-yl]-2-methyl-propyl}-benzamide;
(2-{1-[1-(3-Benzyl-7-chloro-4-oxo-4H-chromen-2-yl)-2-methyl-propyl]-2-p-tolyl-1H-imidazol-4-yl}-ethyl)-carbamic acid benzyl ester;
2-[1-(2-Benzo[1,3]dioxol-5-yl-4,4-dimethyl-4,5-dihydro-imidazol-1-yl)-2-methyl-propyl]-3-benzyl-7-cyano-chromen-4-one;
4-Acetyl-N-(3-amino-propyl)-N-[1-(3-benzyl-7-fluoro-4-oxo-4H-chromen-2-yl)-2-methyl-propyl]-benzamide;
N-(3-Amino-propyl)-N-[1-(3-benzyl-7-chloro-4-oxo-4H-chromen-2-yl)-2-methyl-propyl]-3-fluoro-4-methyl-benzamide;
N-(3-Amino-propyl)-N-{1-[7-fluoro-3-(3-methoxy-benzyl)-4-oxo-4H-chromen-2-yl]-2-methyl-propyl}-4-methoxy-benzamide;
Benzo[1,3]dioxole-5-carboxylic acid (3-amino-propyl)-{1-[7-fluoro-3-(3-methoxy-benzyl)-4-oxo-4H-chromen-2-yl]-2-methyl-propyl}-amide;
2-{1-[4-(2-Amino-ethyl)-2-p-tolyl-imidazol-1-yl]-2-methyl-propyl}-3-benzyl-7-chloro-chromen-4-one;
N-(3-Amino-propyl)-N-[1-(3-benzyl-7-hydroxy-4-oxo-4H-chromen-2-yl)-2-methyl-propyl]-3-fluoro-4-methyl-benzamide;
Benzo[1,3]dioxole-5-carboxylic acid (3-amino-propyl)-[1-(3-benzyl-7-chloro-4-oxo-4H-chromen-2-yl)-2-methyl-propyl]-amide;
N-(3-Amino-propyl)-N-[1-(3-benzyl-7-hydroxy-4-oxo-4H-chromen-2-yl)-2-methyl-propyl]-4-methyl-benzamide;
N-(3-Amino-propyl)-N-{1-[7-fluoro-3-(3-methoxy-benzyl)-4-oxo-4H-chromen-2-yl]-2-methyl-propyl}-2-methoxy-acetamide;
N-(3-Amino-propyl)-N-{1-[7-fluoro-3-(3-methoxy-benzyl)-4-oxo-4H-chromen-2-yl]-2-methyl-propyl}-4-methyl-benzamide;
N-(3-Amino-propyl)-N-[(3-benzyl-7-chloro-4-oxo-4H-chromen-2-yl)-cyclopropyl-methyl]-4-methyl-benzamide;
2,3-Dihydro-benzo[1,4]dioxine-6-carboxylic acid (3-amino-propyl)-[1-(3-benzyl-7-chloro-4-oxo-4H-chromen-2-yl)-2-methyl-propyl]-amide;
N-(3-Amino-propyl)-3-fluoro-N-{1-[7-cyano-3-(3-methoxy-benzyl)-4-oxo-4H-chromen-2-yl]-2-methyl-propyl}-4-methyl-benzamide;
3-Benzyl-7-chloro-2-[2-methyl-1-(2-p-tolyl-4,5-dihydro-imidazol-1-yl)-propyl]-chromen-4-one;
3-Benzyl-7-fluoro-2-{1-[2-(3-fluoro-4-methyl-phenyl)-4,5-dihydro-imidazol-1-yl]-2-methyl-propyl}-chromen-4-one;
2-[1-(4-Aminomethyl-2-p-tolyl-imidazol-1-yl)-2-methyl-propyl]-3-benzyl-7-chloro-chromen-4-one;
3-Benzyl-7-methoxy-2-[2-methyl-1-(2-p-tolyl-4,5-dihydro-imidazol-1-yl)-propyl]-chromen-4-one;
2-[1-(2-Benzo[1,3]dioxol-5-yl-4,5-dihydro-imidazol-1-yl)-2-methyl-propyl]-3-benzyl-7-chloro-chromen-4-one;
4-Acetyl-N-(3-amino-propyl)-N-[1-(3-benzyl-7-chloro-4-oxo-4H-chromen-2-yl)-2-methyl-propyl]-benzamide;
4-Acetyl-N-(3-amino-propyl)-N-{1-[7-cyano-3-(3-methoxy-benzyl)-4-oxo-4H-chromen-2-yl]-2-methyl-propyl}-benzamide;
3-Benzyl-7-chloro-2-{1-[2-(3-fluoro-4-methyl-phenyl)-4,5-dihydro-imidazol-1-yl]-2-methyl-propyl}-chromen-4-one;
3-Benzyl-7-fluoro-2-[2-methyl-1-(2-p-tolyl-4,5-dihydro-imidazol-1-yl)-propyl]-chromen-4-one;
2-[1-(2-Benzo[1,3]dioxol-5-yl-4,5-dihydro-imidazol-1-yl)-2-methyl-propyl]-3-benzyl-7-fluoro-chromen-4-one;
N-(3-Amino-propyl)-N-{1-[7-cyano-3-(3-methoxy-benzyl)-4-oxo-4H-chromen-2-yl]-2-methyl-propyl}-4-methyl-benzamide;
Benzo[1,3]dioxole-5-carboxylic acid (3-amino-propyl)-{1-[7-cyano-3-(3-methoxy-benzyl)-4-oxo-4H-chromen-2-yl]-2-methyl-propyl}-amide;
3-(2-{1-[2-(3-Fluoro-4-methyl-phenyl)-4,5-dihydro-imidazol-1-yl]-2-methyl-propyl}-7-methoxy-4-oxo-4H-chromen-3-ylmethyl)-benzonitrile;
3-{7-Chloro-2-[2-methyl-1-(2-p-tolyl-4,5-dihydro-imidazol-1-yl)-propyl]-4-oxo-4H-chromen-3-ylmethyl}-benzonitrile;
3-{7-Methoxy-2-[2-methyl-1-(2-p-tolyl-4,5-dihydro-imidazol-1-yl)-propyl]-4-oxo-4H-chromen-3-ylmethyl}-benzonitrile;
3-{7-Fluoro-2-[2-methyl-1-(2-p-tolyl-4,5-dihydro-imidazol-1-yl)-propyl]-4-oxo-4H-chromen-3-ylmethyl}-benzonitrile;
N-(3-Amino-propyl)-N-{1-[7-chloro-3-(3-methoxy-benzyl)-4-oxo-4H-chromen-2-yl]-2-methyl-propyl}-3-fluoro-4-methyl-benzamide;
N-(3-Amino-propyl)-N-{1-[3-(3-cyano-benzyl)-7-methoxy-4-oxo-4H-chromen-2-yl]-2-methyl-propyl}-4-methyl-benzamide;
N-(3-Amino-propyl)-N-{1-[3-(3-cyano-benzyl)-7-methoxy-4-oxo-4H-chromen-2-yl]-2-methyl-propyl}-3-fluoro-4-methyl-benzamide;
3-{2-[1-(2-Benzo[1,3]dioxol-5-yl-4,5-dihydro-imidazol-1-yl)-2-methyl-propyl]-7-fluoro-4-oxo-4H-chromen-3-ylmethyl}-benzonitrile;
3-Benzyl-2-{1-[2-(3-fluoro-4-methyl-phenyl)-4,5-dihydro-imidazol-1-yl]-2-methyl-propyl}-4-oxo-4H-chromene-7-carbonitrile;
2-[1-(2-Benzo[1,3]dioxol-5-yl-4,5-dihydro-imidazol-1-yl)-2-methyl-propyl]-3-benzyl-4-oxo-4H-chromene-7-carbonitrile;
3-(7-Chloro-2-{1-[2-(3-fluoro-4-methyl-phenyl)-4,5-dihydro-imidazol-1-yl]-2-methyl-propyl}-4-oxo-4H-chromen-3-ylmethyl)-benzonitrile;
3-{2-[1-(2-Benzo[1,3]dioxol-5-yl-4,5-dihydro-imidazol-1-yl)-2-methyl-propyl]-7-methoxy-4-oxo-4H-chromen-3-ylmethyl}-benzonitrile;
3-(7-Fluoro-2-{1-[2-(3-fluoro-4-methyl-phenyl)-4,5-dihydro-imidazol-1-yl]-2-methyl-propyl}-4-oxo-4H-chromen-3-ylmethyl)-benzonitrile;
Benzo[1,3]dioxole-5-carboxylic acid (3-amino-propyl)-{1-[7-cyano-3-(3-cyano-benzyl)-4-oxo-4H-chromen-2-yl]-2-methyl-propyl}-amide;
Benzo[1,3]dioxole-5-carboxylic acid (3-amino-propyl)-{1-[3-(3-cyano-benzyl)-7-methoxy-4-oxo-4H-chromen-2-yl]-2-methyl-propyl}-amide;
Benzo[1,3]dioxole-5-carboxylic acid (3-amino-propyl)-{1-[3-(3-cyano-benzyl)-7-fluoro-4-oxo-4H-chromen-2-yl]-2-methyl-propyl}-amide;
N-(3-Amino-propyl)-N-{1-[3-(3-cyano-benzyl)-7-fluoro-4-oxo-4H-chromen-2-yl]-2-methyl-propyl}-4-methyl-benzamide;
Benzo[1,3]dioxole-5-carboxylic acid (3-amino-propyl)-{1-[7-chloro-3-(3-methoxy-benzyl)-4-oxo-4H-chromen-2-yl]-2-methyl-propyl}-amide;
N-(3-Amino-propyl)-N-{1-[7-cyano-3-(3-cyano-benzyl)-4-oxo-4H-chromen-2-yl]-2-methyl-propyl}-4-methyl-benzamide;
3-Benzyl-2-[2-methyl-1-(2-p-tolyl-4,5-dihydro-imidazol-1-yl)-propyl]-4-oxo-4H-chromene-7-carbonitrile;
2-[1-(2-Benzo[1,3]dioxol-5-yl-4,5-dihydro-imidazol-1-yl)-2-methyl-propyl]-3-benzyl-7-methoxy-chromen-4-one;
N-(3-Amino-propyl)-N-{1-[7-chloro-3-(3-methoxy-benzyl)-4-oxo-4H-chromen-2-yl]-2-methyl-propyl}-2-methoxy-acetamide;
N-(3-Amino-propyl)-N-{1-[7-cyano-3-(3-cyano-benzyl)-4-oxo-4H-chromen-2-yl]-2-methyl-propyl}-3-fluoro-4-methyl-benzamide;
N-(3-Amino-propyl)-N-{1-[3-(3-cyano-benzyl)-7-fluoro-4-oxo-4H-chromen-2-yl]-2-methyl-propyl}-3-fluoro-4-methyl-benzamide;
2-{1-[4-(2-Amino-ethyl)-2-p-tolyl-imidazol-1-yl]-2-methyl-propyl}-7-chloro-3-(3-methoxy-benzyl)-chromen-4-one;
2-{1-[4-(2-Amino-ethyl)-2-(3-fluoro-4-methyl-phenyl)-imidazol-1-yl]-2-methyl-propyl}-3-benzyl-7-fluoro-chromen-4-one;
2-{1-[4-(2-Amino-ethyl)-2-(3-fluoro-4-methyl-phenyl)-imidazol-1-yl]-2-methyl-propyl}-3-benzyl-4-oxo-4H-chromene-7-carbonitrile;
2-{1-[4-(2-Amino-ethyl)-2-p-tolyl-imidazol-1-yl]-2-methyl-propyl}-3-benzyl-7-fluoro-chromen-4-one;
2-{1-[4-(2-Amino-ethyl)-2-(3-fluoro-4-methyl-phenyl)-imidazol-1-yl]-2-methyl-propyl}-7-chloro-3-(3-methoxy-benzyl)-chromen-4-one;
2-{1-[4-(2-Amino-ethyl)-2-(3-fluoro-4-methyl-phenyl)-imidazol-1-yl]-2-methyl-propyl}-3-benzyl-7-chloro-chromen-4-one;
3-Benzyl-2-{1-[2-(3-fluoro-4-methyl-phenyl)-4,5-dihydro-imidazol-1-yl]-2-methyl-propyl}-7-methoxy-chromen-4-one;
3-(2-{1-[4-(2-Amino-ethyl)-2-p-tolyl-imidazol-1-yl]-2-methyl-propyl}-7-chloro-4-oxo-4H-chromen-3-ylmethyl)-benzonitrile;
3-(2-{1-[4-(2-Amino-ethyl)-2-(3-fluoro-4-methyl-phenyl)-imidazol-1-yl]-2-methyl-propyl}-7-chloro-4-oxo-4H-chromen-3-ylmethyl)-benzonitrile;
2-{1-[4-(2-Amino-ethyl)-2-p-tolyl-imidazol-1-yl]-2-methyl-propyl}-7-fluoro-3-(3-methoxy-benzyl)-chromen-4-one;
2-{1-[4-(2-Amino-ethyl)-2-(3-fluoro-4-methyl-phenyl)-imidazol-1-yl]-2-methyl-propyl}-3-(3-cyano-benzyl)-4-oxo-4H-chromene-7-carbonitrile;
N-(3-Amino-propyl)-3-fluoro-N-{1-[7-hydroxy-3-(3-methoxy-benzyl)-4-oxo-4H-chromen-2-yl]-2-methyl-propyl}-4-methyl-benzamide;
2-{1-[4-(2-Amino-ethyl)-2-p-tolyl-imidazol-1-yl]-2-methyl-propyl}-3-(3-methoxy-benzyl)-4-oxo-4H-chromene-7-carbonitrile;
N-(3-Amino-propyl)-N-[1-(3-benzyl-7-cyano-4-oxo-4H-chromen-2-yl)-2-methyl-propyl]-4-ethoxy-benzamide;
N-(3-Amino-propyl)-N-[1-(3-benzyl-7-cyano-4-oxo-4H-chromen-2-yl)-2-methyl-propyl]-6-trifluoromethyl-nicotinamide;
N-(3-Amino-propyl)-N-[1-(3-benzyl-7-fluoro-4-oxo-4H-chromen-2-yl)-2-methyl-propyl]-6-trifluoromethyl-nicotinamide;
N-(3-Amino-propyl)-N-[1-(3-benzyl-7-fluoro-4-oxo-4H-chromen-2-yl)-2-methyl-propyl]-isonicotinamide;
N-(3-Amino-propyl)-N-[1-(3-benzyl-7-chloro-4-oxo-4H-chromen-2-yl)-2-methyl-propyl]-4-cyano-benzamide;
4-Acetylamino-N-(3-amino-propyl)-N-[1-(3-benzyl-7-chloro-4-oxo-4H-chromen-2-yl)-2-methyl-propyl]-benzamide;
N-(3-Amino-propyl)-N-[1-(3-benzyl-7-chloro-4-oxo-4H-chromen-2-yl)-2-methyl-propyl]-6-trifluoromethyl-nicotinamide;
Benzo[1,2,3]thiadiazole-5-carboxylic acid (3-amino-propyl)-[1-(3-benzyl-7-chloro-4-oxo-4H-chromen-2-yl)-2-methyl-propyl]-amide;
Benzo[1,2,3]thiadiazole-5-carboxylic acid (3-amino-propyl)-[1-(3-benzyl-7-fluoro-4-oxo-4H-chromen-2-yl)-2-methyl-propyl]-amide;
4-Acetylamino-N-(3-amino-propyl)-N-[1-(3-benzyl-7-fluoro-4-oxo-4H-chromen-2-yl)-2-methyl-propyl]-benzamide;
Benzo[1,2,3]thiadiazole-5-carboxylic acid (3-amino-propyl)-[1-(3-benzyl-7-fluoro-4-oxo-4H-chromen-2-yl)-2-methyl-propyl]-amide;
N-(3-Amino-propyl)-N-[1-(3-benzyl-7-chloro-4-oxo-4H-chromen-2-yl)-2-methyl-propyl]-nicotinamide;
N-(3-Amino-propyl)-N-[1-(3-benzyl-7-fluoro-4-oxo-4H-chromen-2-yl)-2-methyl-propyl]-4-methoxy-benzamide;
Benzo[1,2,3]thiadiazole-5-carboxylic acid (3-amino-propyl)-[1-(3-benzyl-7-cyano-4-oxo-4H-chromen-2-yl)-2-methyl-propyl]-amide;
5-Methyl-pyrazine-2-carboxylic acid (3-amino-propyl)-[1-(3-benzyl-7-chloro-4-oxo-4H-chromen-2-yl)-2-methyl-propyl]-amide;
N-(3-Amino-propyl)-N-[1-(3-benzyl-7-cyano-4-oxo-4H-chromen-2-yl)-2-methyl-propyl]-3-dimethylamino-benzamide;
2-[1-(2-Benzo[1,3]dioxol-5-yl-4,5-dihydro-imidazol-1-yl)-2-methyl-propyl]-3-(3-methoxy-benzyl)-4-oxo-4H-chromene-7-carbonitrile;
7-Chloro-3-(3-methoxy-benzyl)-2-[2-methyl-1-(2-p-tolyl-4,5-dihydro-imidazol-1-yl)-propyl]-chromen-4-one;
7-Chloro-2-{1-[2-(3-fluoro-4-methyl-phenyl)-4,5-dihydro-imidazol-1-yl]-2-methyl-propyl}-3-(3-methoxy-benzyl)-chromen-4-one;
2-[1-(2-Benzo[1,3]dioxol-5-yl-4,5-dihydro-imidazol-1-yl)-2-methyl-propyl]-7-chloro-3-(3-methoxy-benzyl)-chromen-4-one;
7-Fluoro-3-(3-methoxy-benzyl)-2-[2-methyl-1-(2-p-tolyl-4,5-dihydro-imidazol-1-yl)-propyl]-chromen-4-one;
7-Fluoro-2-{1-[2-(3-fluoro-4-methyl-phenyl)-4,5-dihydro-imidazol-1-yl]-2-methyl-propyl}-3-(3-methoxy-benzyl)-chromen-4-one;
2-{1-[4-(2-Amino-ethyl)-2-(3-fluoro-4-methyl-phenyl)-imidazol-1-yl]-2-methyl-propyl}-7-fluoro-3-(3-methoxy-benzyl)-chromen-4-one;
2-{(1-[4-(2-Acetylamino-ethyl)-2-(3-fluoro-4-methyl-phenyl)-imidazol-1-yl]-2-methyl-propyl}-3-(3-methoxy-benzyl)-4-oxo-4H-chromene-7-carboxylic acid amide;
3-(2-{1-[4-(2-Amino-ethyl)-2-(3-fluoro-4-methyl-phenyl)-imidazol-1-yl]-2-methyl-propyl}-7-fluoro-4-oxo-4H-chromen-3-ylmethyl)-benzonitrile;
N-{1-[1-(3-Benzyl-7-chloro-4-oxo-4H-chromen-2-yl)-2-methyl-propyl]-2-p-tolyl-1H-imidazol-4-ylmethyl}-acetamide;
Benzo[b]thiophene-2-carboxylic acid (3-amino-propyl)-[1-(3-benzyl-7-cyano-4-oxo-4H-chromen-2-yl)-2-methyl-propyl]-amide;
1-Methyl-1H-indole-2-carboxylic acid (3-amino-propyl)-[1-(3-benzyl-7-cyano-4-oxo-4H-chromen-2-yl)-2-methyl-propyl]-amide;
5-tert-Butyl-2-methyl-2H-pyrazole-3-carboxylic acid (3-amino-propyl)-[1-(3-benzyl-7-cyano-4-oxo-4H-chromen-2-yl)-2-methyl-propyl]-amide;
2,5-Dimethyl-2H-pyrazole-3-carboxylic acid (3-amino-propyl)-[1-(3-benzyl-7-cyano-4-oxo-4H-chromen-2-yl)-2-methyl-propyl]-amide;
5-tert-Butyl-2-methyl-2H-pyrazole-3-carboxylic acid (3-amino-propyl)-[1-(3-benzyl-7-fluoro-4-oxo-4H-chromen-2-yl)-2-methyl-propyl]-amide;
2,5-Dimethyl-2H-pyrazole-3-carboxylic acid (3-amino-propyl)-[1-(3-benzyl-7-chloro-4-oxo-4H-chromen-2-yl)-2-methyl-propyl]-amide;
2,5-Dimethyl-2H-pyrazole-3-carboxylic acid (3-amino-propyl)-[1-(3-benzyl-7-fluoro-4-oxo-4H-chromen-2-yl)-2-methyl-propyl]-amide;
5-Methyl-2H-pyrazole-3-carboxylic acid (3-amino-propyl)-[1-(3-benzyl-7-cyano-4-oxo-4H-chromen-2-yl)-2-methyl-propyl]-amide;
5-Methyl-2H-pyrazole-3-carboxylic acid (3-amino-propyl)-[1-(3-benzyl-7-fluoro-4-oxo-4H-chromen-2-yl)-2-methyl-propyl]-amide;
Furan-2-carboxylic acid (3-amino-propyl)-[1-(3-benzyl-7-fluoro-4-oxo-4H-chromen-2-yl)-2-methyl-propyl]-amide;
Furan-2-carboxylic acid (3-amino-propyl)-[1-(3-benzyl-7-chloro-4-oxo-4H-chromen-2-yl)-2-methyl-propyl]-amide;
2,5-Dimethyl-furan-3-carboxylic acid (3-amino-propyl)-[1-(3-benzyl-7-cyano-4-oxo-4H-chromen-2-yl)-2-methyl-propyl]-amide;
2,5-Dimethyl-furan-3-carboxylic acid (3-amino-propyl)-[1-(3-benzyl-7-fluoro-4-oxo-4H-chromen-2-yl)-2-methyl-propyl]-amide;
5-Methyl-thiophene-2-carboxylic acid (3-amino-propyl)-[1-(3-benzyl-7-cyano-4-oxo-4H-chromen-2-yl)-2-methyl-propyl]-amide;
5-Methyl-thiophene-2-carboxylic acid (3-amino-propyl)-[1-(3-benzyl-7-fluoro-4-oxo-4H-chromen-2-yl)-2-methyl-propyl]-amide;
5-Methyl-isoxazole-3-carboxylic acid (3-amino-propyl)-[1-(3-benzyl-7-chloro-4-oxo-4H-chromen-2-yl)-2-methyl-propyl]-amide;
5-Methyl-2-trifluoromethyl-furan-3-carboxylic acid (3-amino-propyl)-[1-(3-benzyl-7-cyano-4-oxo-4H-chromen-2-yl)-2-methyl-propyl]-amide;
5-Methyl-isoxazole-3-carboxylic acid (3-amino-propyl)-[1-(3-benzyl-7-fluoro-4-oxo-4H-chromen-2-yl)-2-methyl-propyl]-amide;
5-Methyl-isoxazole-3-carboxylic acid (3-amino-propyl)-[1-(3-benzyl-7-cyano-4-oxo-4H-chromen-2-yl)-2-methyl-propyl]-amide;
Benzo[c]isoxazole-3-carboxylic acid (3-amino-propyl)-[1-(3-benzyl-7-chloro-4-oxo-4H-chromen-2-yl)-2-methyl-propyl]-amide;
Benzo[c]isoxazole-3-carboxylic acid (3-amino-propyl)-[1-(3-benzyl-7-fluoro-4-oxo-4H-chromen-2-yl)-2-methyl-propyl]-amide;
1-Methyl-1H-pyrrole-2-carboxylic acid (3-amino-propyl)-[1-(3-benzyl-7-fluoro-4-oxo-4H-chromen-2-yl)-2-methyl-propyl]-amide;
1-Methyl-1H-imidazole-4-carboxylic acid (3-amino-propyl)-[(R)-1-(3-benzyl-7-cyano-4-oxo-4H-chromen-2-yl)-2-methyl-propyl]-amide;
N-(3-Amino-propyl)-N—[(R)-1-(3-benzyl-7-cyano-4-oxo-4H-chromen-2-yl)-2-methyl-propyl]-3-dimethylamino-benzamide;
5-Methyl-2-trifluoromethyl-furan-3-carboxylic acid (3-amino-propyl)-[(R)-1-(3-benzyl-7-cyano-4-oxo-4H-chromen-2-yl)-2-methyl-propyl]-amide;
5-Methyl-isoxazole-3-carboxylic acid (3-amino-propyl)-[(R)-1-(3-benzyl-7-cyano-4-oxo-4H-chromen-2-yl)-2-methyl-propyl]-amide;
1-Methyl-1H-imidazole-4-carboxylic acid (3-amino-propyl)-[(R)-1-(3-benzyl-7-fluoro-4-oxo-4H-chromen-2-yl)-2-methyl-propyl]-amide;
1-Methyl-1H-pyrrole-2-carboxylic acid (3-amino-propyl)-[(R)-1-(3-benzyl-7-fluoro-4-oxo-4H-chromen-2-yl)-2-methyl-propyl]-amide;
Benzo[c]isoxazole-3-carboxylic acid (3-amino-propyl)-[(R)-1-(3-benzyl-7-fluoro-4-oxo-4H-chromen-2-yl)-2-methyl-propyl]-amide; and
5-Methyl-isoxazole-3-carboxylic acid (3-amino-propyl)-[(R)-1-(3-benzyl-7-fluoro-4-oxo-4H-chromen-2-yl)-2-methyl-propyl]-amide, and

pharmaceutically acceptable salts thereof,

[b] a chemotherapeutic agent selected from doxorubucin, cisplatin, 5-fluoruracil, gemcitabine, irinotecan, docetaxel, capecitabine and carboplatin.

In another embodiment the pharmaceutically acceptable salt of a compound of Formula (I) is a hydrochloride salt.

In one embodiment the combination therapy method for treating cellular proliferative diseases in a mammal in need thereof comprises administration to said mammal of N-(3-Aminopropyl)-N—[(R)-1-3-benzyl-7-chloro-4-oxo-4H-chromen-2-yl-2-methyl-propyl]-4-methyl-benzamide, or a pharmaceutically acceptable salt thereof (e.g., hydrochloride), in combination with doxorubicin, cisplatin, gemcitabine, irinotecan, carboplatin, docetaxel, or capecitabine. The N-(3-Aminopropyl)-N—[(R)-1-3-benzyl-7-chloro-4-oxo-4H-chromen-2-yl-2-methyl-propyl]-4-methyl-benzamide or its pharmaceutically acceptable salt and the other chemotherapeutic agent may be administered in the form of a pharmaceutical composition such as described herein, either in separate compositions or in the same composition.

In a particular embodiment, the combination therapy method for treating cellular proliferative diseases in a mammal in need thereof comprises administration of N-(3-Aminopropyl)-N—[(R)-1-3-benzyl-7-chloro-4-oxo-4H-chromen-2-yl-2-methyl-propyl]-4-methyl-benzamide or a pharmaceutically acceptable salt thereof (e.g., the hydrochloride salt) in combination with G-CSF.

In a particular embodiment, the combination therapy method for treating cellular proliferative diseases in a mammal in need thereof comprises administration of N-(3-Aminopropyl)-N—[(R)-1-3-benzyl-7-chloro-4-oxo-4H-chromen-2-yl-2-methyl-propyl]4-methyl-benzamide or a pharmaceutically acceptable salt thereof (e.g., the hydrochloride salt) in combination with doxorubicin.

In another particular embodiment, the combination therapy method for treating cellular proliferative diseases in a mammal in need thereof comprises administration of N-(3-Aminopropyl)-N—[(R)-1-3-benzyl-7-chloro-4-oxo-4H-chromen-2-yl-2-methyl-propyl]-4-methyl-benzamide or a pharmaceutically acceptable salt thereof (e.g., the hydrochloride salt) in combination with cisplatin.

In another particular embodiment, the combination therapy method for treating cellular proliferative diseases in a mammal in need thereof comprises administration of N-(3-Aminopropyl)-N—[(R)-1-3-benzyl-7-chloro-4-oxo-4H-chromen-2-yl-2-methyl-propyl]-4-methyl-benzamide or a pharmaceutically acceptable salt thereof (e.g., the hydrochloride salt) in combination with gemcitabine.

In another particular embodiment, the combination therapy method for treating cellular proliferative diseases in a mammal in need thereof comprises administration of N-(3-Aminopropyl)-N—[(R)-1-3-benzyl-7-chloro-4-oxo-4H-chromen-2-yl-2-methyl-propyl]-4-methyl-benzamide or a pharmaceutically acceptable salt thereof (e.g., the hydrochloride salt) in combination with irinotecan.

In another particular embodiment, the combination therapy method for treating cellular proliferative diseases in a mammal in need thereof comprises administration N-(3-Aminopropyl)-N—[(R)-1-3-benzyl-7-chloro-4-oxo-4H-chromen-2-yl-2-methyl-propyl]-4-methyl-benzamide or a pharmaceutically acceptable salt thereof (e.g., the hydrochloride salt) in combination with carboplatin.

In another particular embodiment, the combination therapy method for treating cellular proliferative diseases in a mammal in need thereof comprises administration of N-(3-Aminopropyl)-N—[(R)-1-3-benzyl-7-chloro-4-oxo-4H-chromen-2-yl-2-methyl-propyl]-4-methyl-benzamide or a pharmaceutically acceptable salt thereof (e.g., the hydrochloride salt) in combination with docetaxel.

In another particular embodiment, the combination therapy method for treating cellular proliferative diseases in a mammal in need thereof comprises administration of N-(3-Aminopropyl)-N—[(R)-1-3-benzyl-7-chloro-4-oxo-4H-chromen-2-yl-2-methyl-propyl]-4-methyl-benzamide or a pharmaceutically acceptable salt thereof (e.g., the hydrochloride salt) in combination with capecitabine.

In another particular embodiment, the combination therapy method for treating cellular proliferative diseases in a mammal in need thereof comprises administration of N-(3-aminopropyl)-N—[(R)-1-3-benzyl-7-chloro-4-oxo-4H-chromen-2-yl-2-methyl-propyl]-4-methyl-benzamide or a pharmaceutically acceptable salt thereof (e.g., the hydrochloride salt) in combination with a proteasome inhibitor, such as bortezomib. In certain embodiments, the N-(3-aminopropyl)-N—[(R)-1-3-benzyl-7-chloro-4-oxo-4H-chromen-2-yl-2-methyl-propyl]-4-methyl-benzamide or its pharmaceutically acceptable salt is administered prior to the proteasome inhibitor, such as at least about 24 hours prior to administration of the proteasome inhibitor. In certain embodiments, the N-(3-aminopropyl)-N—[(R)-1-3-benzyl-7-chloro-4-oxo-4H-chromen-2-yl-2-methyl-propyl]-4-methyl-benzamide or its pharmaceutically acceptable salt is administered simultaneously with the proteasome inhibitor.

The Examples set forth below are illustrative of the present invention and are not intended to limit, in any way, the scope of the present invention.

### EXAMPLES

### Example 1

N-(3-Aminopropyl)-N—[(R)-1-3-benzyl-7-chloro-4-oxo-4H-chromen-2-yl-2-methyl-propyl]-4-methyl-benzamide or its hydrochloride salt (hereinafter “Compound A”) is an example of a potent cytotoxic chromenone compound. Compound A demonstrates efficacy on an intermittent schedule in a spectrum of preclinical murine syngeneic tumor models, which include chemorefractory models.

Compound A has been evaluated in several different tumor models, including four human tumor xenografts in nude mice, and one syngeneic mouse tumor model. Significant efficacy was observed in all but one of these models, with the most sensitive tumors responding with regressions at doses of Compound A as low as 25% of the maximum tolerated dose (MTD).

In order to investigate the effects of dosing frequency of Compound A, the murine P388 lymphocytic leukaemia was used. Compound A was administered intraperitoneally either daily for 9 days (q1d×9), every second day for 5 doses (q2d×5), every third day for 4 doses (q3d×4), every fourth day for 3 doses (q4d×3), or on Days 2 and 10 (q8d×2) at multiple dose levels on each schedule. These studies indicated that a q4d×3 schedule was optimum. More frequent administration of the compound was poorly tolerated and dictated a lower total dose. On the q4d×3 schedule, Compound A demonstrated good dose dependent efficacy against P388 lymphocytic leukaemia, resulting in a mean (±SEM) % increase in lifespan (ILS) of 156±10 and a net cell kill (NCK) of 2.9±0.5 logs at the MTD. At doses equivalent to either ¼ or ½ the MTD, % ILS and NCK were reduced.

The anti-tumor activity of Compound A was also evaluated in four murine xenograft tumor models. Compound A was administered intraperitoneally on a q4d×3 schedule at dose levels of 1.25, 2.5, 5, 10, 20 or 40 mg/kg. Anti-tumor activity was assessed by tumor growth delay (time differential between control and treated mice reaching a tumor volume of 1000 mm³; T-C₁₀₀₀), and complete or partial regression.

Colo205, a fast growing colon carcinoma xenograft was very sensitive to Compound A; dose-dependent anti-tumor activity was observed in this model and complete tumor regressions were observed at the MTD. Partial tumor regressions were observed in the majority of animals treated with doses as low as 25% of the MTD. HT 29, a chemo-refractory colon carcinoma, was only partially sensitive to Compound A, such that only a delay in tumor growth (T-C₁₀₀₀=25 days) was observed. Compound A did not induce any tumor regressions in the tubulin-agent-sensitive mammary carcinoma, MX-1. In this study, Compound A delayed tumor growth (T-C₁₀₀₀) by 10 days. The human lung carcinoma, MV 522, was refractory to Compound A up to its MTD.

In summary, efficacy was observed against Colo205 colon carcinoma, HT-29 colon carcinoma and MX-1 breast carcinoma. Colo205 was the most sensitive tumor tested while MV 522 lung carcinoma was refractory to treatment with Compound A.

### Example 2

Preclinical toxicology studies were conducted with Compound A in rats and dogs.

Dogs were administered Compound A by 1-hr IV infusion. Doses up to 40 mg/m² were tested in a single dose-range finding study and doses of 5, 10 and 20 mg/m² were tested in a 3-week (once weekly) toxicology study. Drug-related findings occurred at all dose levels, but severity and incidence were generally dose-proportional. One of 10 dogs was euthanized in moribund condition on Day 4 after receiving a single 1-hr IV infusion of 20 mg/m². In dogs given ≧10 mg/m², clinical observations of gastrointestinal (GI) disturbances included vomiting/retching, fecal abnormalities, hypoactivity, anorexia and body weight losses. There were effects on hematopoietic cells (hematologic and/or microscopic) at all doses, and testicular germinal epithelial abnormalities were observed in males given 20 mg/m². All drug-related findings showed evidence of reversibility two weeks after dosing except lymphoid depletion in the thymus and testicular germinal epithelial abnormalities. Based on these findings, the highest non-severely toxic dose in the dog is 10 mg/m².

Rats were administered Compound A by 1 hr (6, 18 mg/m²), 6 hr (36, 72, 108 mg/m²) and 24-hr (6, 12, 18, 36, 72 mg/m²) IV infusions. Transient drug-related findings occurred at all doses, but severity and incidence were generally dose proportional and dependent on the duration of infusion. Most consistent findings included hematopoeitic toxicity (depletion in bone marrow and thymus and associated hematologic changes) and enteropathy (necrosis/regeneration of intestinal crypt epithelium and villus atrophy and body weight loss or decrease in body weight gain). Microscopic changes (necrosis, apoptosis and/or cellular degeneration) were also evident in liver, mammary glands, testes and epididymus, and at ≧36 mg/m², increased mitotic figures in many tissues. All drug-related toxicities completely reversed 2 weeks after dosing, with the exception of degenerative testicular changes at ≧18 mg/m². In the 1 to 6-hr infusion studies, there was no mortality, however based on the severity of hematopoietic and gastrointestinal toxicities, the MTD was 72 mg/m². When the infusion was increased to 24-hr, mortality occurred at 18 mg/m² after a single dose and at 12 mg/m² after repeated weekly dosing.

Compound A was hemolytic in vitro when mixed with rat, dog, rabbit and human blood at concentrations ≧0.6 mg/mL, while at 0.3 mg/mL no hemolysis was observed. Hemolysis was not observed in any of the in vivo studies, nor has it been observed in the ongoing Phase I trial.

Compound A did not show evidence of genotoxic activity in in vitro Ames test or mouse lymphoma mutagenicity assays, but did show positive results as an aneugen in an in vivo rat micronucleus study at all doses tested (≧3 mg/m² administered daily×2). Positive findings are consistent with the expected pharmacology of Compound A.

In summary, the dog proved to be a more sensitive species for preclinical safety assessment than the rat. The most sensitive Compound A-related toxicities were generally limited to GI disturbances and bone marrow toxicity, consistent with action on proliferative tissues. Drug effects, with the exception of lymphoid depletion and/or testicular degeneration, reversed shortly following treatment discontinuation. There was no histological evidence of neurotoxicity.

### Example 3

Preliminary safety data from the ongoing Compound A dose-escalation Phase I study in patients with solid tumors shows that a schedule of a single, one hour infusion every 21 days can elicit Grade 4 neutropenia lasting ≧5 days at doses of 5 mg/m² and above. Dose limiting toxicities from this study have included prolonged Grade 4 neutropenia (≧5 days), Grade 3 febrile neutropenia with and without infection, Grade 3 elevated transaminases, Grade 3 hyperbilirubinemia and Grade 3 hyponatremia (not thought to be study drug related), while neurotoxicity, mucositis, thrombocytopenia, alopecia, and nausea vomiting requiring pre-medication have not been observed.

Compound A also may be administered at 4 mg/m²; 1 of the first 6 patients treated at 4 mg/m² had Grade 3 hypophosphatemia (not thought to be drug-related), defined as a dose-limiting toxicity by the protocol, regardless of the investigator's assessment of drug-relatedness. In this study, the ANC generally achieved a nadir at Day 7-8 and had completely recovered by Day 15.

In an ongoing Phase 1 Study of the drug given Q21 days to patients with solid tumors (Compound A), the maximum administered dose was 8 mg/m²; an expanded cohort is currently being enrolled at 4 mg/m² as the presumptive maximum tolerated dose (MTD). Prolonged (≧5 days), reversible neutropenia has been the most commonly occurring dose-limiting toxicity with Compound A, with the ANC generally reaching a nadir at Day 7-8 and recovering to baseline by Day 15.

### Example 4

Compound A is also being given to patients with Hodgkin's Disease and Non-Hodgkin's Lymphoma (NHL) as a one-hour intravenous infusion on Days 1 and 15 of a 28 day schedule in the absence of planned prophylactic granulopoetic support and then again in the presence of planned prophylactic granulopoetic support.

A treatment cycle is defined as a 28-day period and the initial starting dose in the Phase I study will be 2 mg/m². Doses will be escalated in 1 mg/m² increments until the MTD is established. Dose escalation will proceed based on the toxicity encountered during the first cycle of treatment.

If the MTD without prophylactic granulopoetic support is determined by neutropenia, dose escalation will continue with planned prophylactic granulopoetic support. The maximum tolerated dose (MTD) will be determined first without prophylactic granulocyte colony stimulating factor (GCSF) support; if the DLT determining this first MTD is neutropenia, a second MTD will be determined with GCSF support. Dosing with GCSF will begin with the MTD established in the absence of prophylactic granulopoetic support and be escalated to a second MTD in increments of 1 mg/m² in accordance with the same safety and tolerability criteria. GCSF will be administered subcutaneously on Days 2, 3, 4, 16, 17, and 18 of each 28-day cycle. Patients ≦70 kg will receive GCSF 300 μg/day; patients >70 kg will receive GCSF 480 μg/day. An example of a dose escalation scheme first without GCSF, then with GCSF, is given below. Evaluation of differences between MTDs of patients receiving vs. patients not receiving prophylactic GCSF will be accomplished by a comparison of clinical information such as, but not limited to; dosing information, CBCs, PK information and adverse events.

### Example Phase I Dose Escalation Scheme

| Cohort | Compound A Dose | GCSF? |
|---|---|---|
| 1 | 2 mg/m² | N |
| 2 | 3 mg/m² | N |
| 3 | 4 mg/m² | N |
| 4 | 5 mg/m² | N |
| 5 | 6 mg/m² | N |

(Continue dose escalation by 1 mg/m² until first MTD* is determined.)

| | | |
|---|---|---|
| 6 | MTD* | Y |
| 7 | MTD* + 1 mg/m² | Y |
| 8 | MTD* + 2 mg/m² | Y |
| 9 | MTD* + 3 mg/m² | Y |
| 10 | MTD* + 4 mg/m² | Y |

(Continue dose escalation by 1 mg/m² until new MTD with GCSF is determined.)
*i.e., MTD determined in the absence of planned prophylactic GCSF

### Example 5

Studies were conducted to determine the compatibility of Compound A Injection, 1 mg/mL as free base, with various diluents and infusion sets. In the first study, the product was diluted in each of the individual diluents (5% mannitol injection, 0.9% sodium chloride injection and 5% dextrose injection) and exposed to the infusion sets (Baxter IntraVia™ 250 mL infusion bag and Abbott 250 mL sterile evacuated glass bottle), assayed after 24 hours and checked visually. The data indicate that there was no loss of Compound A and was visually confirmed as a clear, colorless, particle-free solution. In the second study, the product was diluted in the same individual diluents as in the first study, placed in the Baxter IntraVia™ 250 mL infusion bag with the Alaris Low Sorbing infusion set, passed (250 mL/1 hour) through the infusion apparatus over a one hour time period and assayed. The data indicate that there was no loss with the 5% mannitol injection, and essentially no loss with the 0.9% sodium chloride injection or the 5% dextrose injection diluents. For uniformity across the study only the following infusion components may be used: 5% dextrose injection with the Baxter 250 mL IntraVia™ infusion bag (product #2B8012) and Alaris Low Sorbing infusion set (product #72953).

Before use, each vial should be appropriately diluted to the desired concentration with 5% dextrose IV solution. Each vial of Compound A is intended for single use. Multiple vials may be necessary to administer the proper dose of Compound A.

### Example 6

Pharmacogenetics (PGx) is the study of variability in drug handling or response due to hereditary factors in different populations. There is increasing evidence that an individual's genetic composition (i.e., his or her genotype) may impact the pharmacokinetics, pharmacodynamics, and/or the incidence of adverse events for a given investigational product. Some reported examples of PGx analysis include:

| Drug | Disease | Gene | Outcome |
|---|---|---|---|
| 6-meracap- | Lymphoblastic | S-methyltransferase | Deficiency of the TPMT |
| topurine | Leukemia | | enzyme can be associated |
| (6-MP) | | | with toxicity and severe |
| | | | myelosuppression as |
| | | | patients are not able |
| | | | to sufficiently clear |
| | | | active thioguanine |
| | | | nucleotides [André, 2002; |
| | | | McLeod, 2002]. |
| 5-FU | Colorectal | Dihydropyrimidine | Variants in the DPD gene |
| | Cancer | dehydrogenase | result in little or no DPD |
| | | (DPD) | and predisposition to toxicity |
| | | | to 5-FU [Daisio, 2001; |
| | | | Kawakami, 2001; Mattison, |
| | | | 2002]. |
| Atomoxetine | ADHD | CYP2D6 | Polymorphism in CYP2D6 |
| Desipramine | Depression | | results in different |
| | | | phenotypes: poor, |
| | | | intermediate, or extensive |
| | | | metabolizers. Poor |
| | | | metabolizing genotypes are |
| | | | at risk of drug accumulation |
| | | | and associated toxicity |
| | | | [Belle, 2002; Daly, 1995]. |

Two screening assays have been conducted in order to determine the potential of Compound A to inhibit recombinant human CYP450 enzymes in either a concentration- or time-dependent manner.

In one study, the in vitro concentration-dependent inhibition of CYP3A4, CYP2D6, CYP2C19, CYP1A2 and CYP2C9 was determined by monitoring the metabolism of appropriate probe substrates in the presence and absence of Compound A (0-100 μM) (Report CH2003/00043/00). Compound A demonstrated potent to moderate inhibition of CYP3A4, moderate inhibition of CYP2D6, moderate to weak inhibition of CYP2C19 and weak inhibition of CYP1A2 and CYP2C9.

An additional study was conducted to determine the time-dependent inhibition potential of Compound A by monitoring the metabolism of the same probe substrates in the presence and absence of Compound A (0, 10 or 50 μM) following incubation with CYP1A2, CYP2C9, CYP2D6 or CYP3A4 for 0, 5, 10, 15 or 20 minutes (Report CH2003/00042/00). Compound A did not demonstrate any time-dependent inhibition of CYP1A2, CYP2C9, CYP2D6 or CYP3A4 activities in this study.

Two studies were conducted to determine whether Compound A is a substrate and inhibitor of human P-glycoprotein (Pgp).

In MDCKII-MDR1 cells, Compound A inhibited the basolateral to apical (B→A) transport of a probe substrate (digoxin) with an IC₅₀ of 3.74 μM. The percentages of digoxin transport not inhibited by GF120918A (2 μM; a known inhibitor of Pgp) or Compound A (13.6 μM) were similar (˜30%), and this component most likely represented passive transport. These data also indicate that Compound A (13.6 μM) fully inhibited human Pgp-mediated digoxin transport.

In a study designed to determine if Compound A was a Pgp substrate, the apical efflux ratio (rate of B→A divided by rate of A→B) for transport of ¹⁴C-Compound A (3 μM) was determined in MDCKII-MDR1 cells in the presence and absence GF120918A (2 μM). The apical ratio of ¹⁴C-Compound A was 37.8 and 0.8 in the presence and absence of GF120918A, respectively, thus confirming that Compound A is a Pgp substrate. ¹⁴C-Compound A demonstrated low passive membrane permeability (<50 nm/s) with an average P_{7.4} of 25 nm/s.

If at any time it appears that there is a potential unexpected or unexplained variation in response to or handling of Compound A (e.g., pharmacokinetics, efficacy and/or safety) that may be attributable to genetic variation, then PGx analysis may be conducted. In these circumstances, the analysis undertaken will be limited to PGx analysis of Compound A handling or response and may include the evaluation of specific candidate genes, the conduct of a whole genome single nucleotide polymorphism (SNP) scan or other marker scan.

For the candidate gene approach, the genes of the receptor/enzymes/proteins/transporters mentioned in the section above may be studied. In addition, continuing research may identify other enzymes/transporters/proteins/receptors that may be involved in response to or handling of investigational product. Genes of these enzymes/transporters/proteins/receptors may also be studied.

For the whole genome SNP scan approach, SNP or other genetic marker sets across the genome may be evaluated to identify those markers associated with differential drug handling or response.

Variants in genes in the following list will be considered for blood samples as part of Study Compound A:
Polymorphisms in MDR-1, CYP3A4, CYP2D6, CYP2C19, CYP2C9, and CYP1A2 and their relationship to Cmax and AUC of Compound A.
Polymorphisms in albumin and alpha-1 acid gylcoprotein and their relationship to Cmax and AUC of Compound A and adverse events.

Additional enzymes/transporters/proteins/receptors associated with response to Compound A may be studied. Variants in the genes of these additional enzymes/transporters/proteins/receptors (or their expression) may also be studied on the coded DNA sample.

Generally two approaches will be used to explore genetic variation in drug handling or response.
1. Hypothesis driven approach: A specific hypothesis is generated about sections of DNA (or individual single nucleotide polymorphisms (SNPs) or other genetic markers) that may be associated with differential drug handling or response. Specific sections of DNA may be selected from areas of the genome (e.g., candidate genes) known to encode the drug target, drug metabolizing enzymes, areas associated with mechanisms underlying adverse events, and those linked to study disease and, thus, linked to drug response.
2. Genome-wide approach utilizing polymorphic markers (e.g., SNPs): By evaluating large numbers of polymorphic markers throughout the genome, sets of markers may be identified that correspond to differential drug response or handling.

Analysis of genetic markers (e.g., whether within candidate genes or SNPs studied in a genome-wide analysis) will include the following considerations. The genotypic frequencies of each polymorphism will be evaluated for conformity to those expected under normal conditions by employing Hardy-Weinberg Equilibrium testing. Any departure from expectation will be taken into account, possibly signaling a data error or alternatively a connection between the polymorphism and cancer.

For pairs of polymorphisms, the degree to which alleles from the two sites are correlated (linkage disequilibrium) may also be evaluated. If the genotypes at two polymorphic sites within a gene are shown to be statistically associated with a response to investigational product, the degree of linkage disequilibrium will aid interpretation in that it will indicate the extent to which the two sites are exerting independent effects.

A decision regarding the construction and analysis of marker haplotypes—combinations of alleles from different polymorphic sites that are inherited from one parent—may be guided by the assessment of linkage disequilibrium. For example, if there is no linkage disequilibrium between polymorphic sites, then haplotype construction will be uninformative.

Differences in baseline clinical characteristics and potential contributing covariates may be summarized and compared among genotype (or haplotype) subgroups.

Analyses may be carried out to evaluate the degree of association between patient genotype (or haplotype) and selected parameters (e.g., pharmacokinetics, efficacy and safety). Where such genotypic tests are inappropriate (for example, where the number of marker genotypes is too large and/or the frequency of individual genotypes too small), allelic tests may be conducted. Allelic tests evaluate whether the frequency of each marker allele is the same in responders and non-responders.

In addition to evaluating the main effects of the genotypes (haplotypes or alleles) on the selected parameters, the possibility of a treatment group by genotype (haplotype or allele) interaction may also be explored. If appropriate, the joint effects of multiple markers (gene-gene interactions) may also be evaluated.

### Example 7

Compound A is a potent specific KSP inhibitor currently in Phase II clinical trials. Compound A is a structurally distinct KSP inhibitor with a Ki of 0.1 M and cytotoxic activity at less than 2 nM in a broad spectrum of tumor cell lines. In vitro activities of Compound A and ispinesib is shown below. As a single agent, Compound A exhibits activity against advanced human tumor xenografts Colo205 (complete regressions), MCF-7, SK-MES, H69, OVCAR-3 (complete and partial regressions), and HT-29, MDA-MB-231, A2780 (tumor growth delay)

| | ispinesib | Compound A |
|---|---|---|
| Kinesin Affinity | | |
| Kᵢ (nM) | | |
| Human KSP | 0.6 | 0.1 |
| Mouse KSP | 0.76 | 0.12 |
| Other kinesins | >70,000 | >70,000 |
| Cellular Activity | | |
| (IC₅₀, nm) | | |
| HT29 | 1.2 | 1.9 |
| SKOV3 | 1 | 0.2 |
| Colo205 | 0.18 | 0.07 |
| MV522 | 9.7 | 1.7 |
| MX1 | 2.8 | 0.06 |
| P388 (murine) | 19.7 | 14.4 |

The processes linking mitotic arrest and apoptosis are poorly characterized. In an effort to identify genes that may or enhance response to KSP inhibitor exposure, a library of small interfering RNAs (siRNA) focused on cell cycle regulatory proteins was screened. This screen identified several components of the Anaphase Promoting Complex (APC) E3 ubiquitin ligase, including Cdc27 and Cdc16. Loss of these APC components is expected to impair ubiquitin-dependent proteolysis and block exit from mitosis.

Based upon these findings, the potential utility of combining proteasome inhibitors with KSP inhibitors in vitro and in vivo (KSPi inhibitors: Compound A and Compound B; proteasome inhibitors: cdc27 siRNA and bortezomib) was evaluated.

### Example 8

The methods used in each of the following Examples were as follows.

A Clonogenic viability assay was conducted in which HT29 cells were exposed to drug for the indicated periods. Following treatment, cells were trypsinized, equal proportions of control and drug-treated wells replated in fresh, drug-free medium and colonies counted after 8-12 days of growth.

Next, timelapse microscopy and siRNA transfection were conducted in which SKOV3 cell stably expressing GFP-H2B chimera in 96 wells plates (˜5000 cells/well) were imaged using the ImageExpress Live Cell Imaging System (Molecular Devices) acquiring fluorescence images of GFP-H2B every 15 minutes for 5 days. Transfection of an siRNA pool targeting cdc27 was carried out using Lipofectamine 2000 (Invitrogen) as described by manufacturer. Eighteen (18) hours post-transfection, drugs were added at the indicated concentrations. Timelapse images were quantified using custom software to score number of cells per field over time.

Finally, tolerability and efficacy studies were conducted in which HT29 tumors maintained by serial passage were implanted subcutaneously in 9-10 weeks old female athymic no/nu mice (Harlan) Twenty-one (21) days post-implantation, when tumors reached 63-196 mm³, mice were randomized into cohorts of nine with mean tumor volumes ˜100 mm³. Bortezomib was formulated in sterile saline, Compound A in 2% Cremaphor EL:2% DMA in acidified water and paclitaxel in 5% EtOH:5% Cremaphor EL:90% D5W. Drugs were delivered on the indicated schedules at 10 ml/kg by i.p. injection, except paclitaxel, which was administered by intravenous injection. Tumors were measured twice weekly and mice euthanized when tumor reached 1000 mm³ or at day 59. Mice were weighed twice weekly. Acceptable toxicity was defined as body weight loss of <20% and ≦1 treatment-related death among ten treated animals. Toxicity exceeding these levels was considered above MTD.

### Example 9

KSP inhibitor tumor cell killing is enhanced with siRNA targeting Anaphase Promoting Complex (APC) subunit Cdc27.

Extended timelapse analysis (5 days) of GFP-H2B SKOV3 cells transfected with cdc27 siRNA (t=0) and treated with concentration of KSP inhibitor below single agent effective dose (1 nM Compound B, @ t=18 h). Graphical results showing the extended timelapse analysis are shown in FIG. 1.

### Example 10

Exposure of cells in vitro to Compound B and the proteasome inhibitor bortezomib resulted in increased cancer cell death compared to either single agent in HT29 Clonogenic survival assays.

Sequenced exposure of HT29 colon carcinoma cells with Compound B and bortezomib was initiated, with administration of 10 nM of Compound B (0-24 hours) followed by bortezomib (24-48 hours). Graphical results of the comparison of 5 nM Compound B and 10 nM mg/kg bortezomib as single agents or in combination is shown in FIG. 2.

In summary, exposure of cells to the KSP inhibitor Compound B and the proteasome inhibitor bortezomib resulted in increased kill compared to either single agent at this concentration in HT29 Clonogenic survival assays.

### Example 11

Exposure of cells in vivo to Compound A and the proteasome inhibitor bortezomib either administered simultaneously or separated by 24 hours in both orders of administration resulted in increased cancer cell death compared to bortezomib monotherapy when dosed simultaneously or with Compound A first. Dosing of bortezomib prior to Compound A was not significantly different from either monotherapy.

Combination studies with Compound A and bortezomib in nude mice were initiated, administering both drugs on q4d×3 schedule simultaneously and separated by 24 hours in both orders of addition. The single does MTD (q4d×3) of Compound A was about 10 mg/kg and for bortezomib was about 1.5 mg/kg.

Anti-tumor activity of sequenced (24 hour offset, q4d×3) and simultaneous administration was explored in HT29 tumor xenografts. Graphical results of the in vivo tolerability of 4.5 mg/kg Compound A and 1.5 mg/kg bortezomib as single agents or in combination is shown in FIG. 3.

In summary, exposure of cells to the KSP inhibitor Compound A followed by the proteasome inhibitor bortezomib 24 hours later resulted in increased cancer cell death compared to the reverse order of addition. The combination had activity that was superior to bortezomib monotherapy when dosed simultaneously or with Compound A first. Dosing of bortezomib prior to Compound A was not significantly different from either monotherapy.

### Example 12

Exposure of cells in vitro to Compound A and the proteasome inhibitor bortezomib either administered simultaneously or separated by 24 hours in both orders of administration resulted in increased cancer cell death compared to bortezomib monotherapy when dosed simultaneously or with Compound A first. Dosing of bortezomib prior to Compound A was not significantly different from either monotherapy.

Combination studies with Compound A and bortezomib in nude mice were initiated, administering both drugs on q4d×3 schedule simultaneously and separated by 24 hours in both orders of addition.

Anti-tumor activity of sequenced (24 hour offset, q4d×3) and simultaneous administration was explored in HT29 tumor xenografts. Graphical results of the comparison of 4.5 mg/kg Compound A and 1.5 mg/kg bortezomib as single agents or in combination is shown in FIG. 4.

In summary, exposure of cells to the KSP inhibitor Compound A followed by the proteasome inhibitor bortezomib 24 hours later resulted in increased cancer cell death compared to the reverse order of addition. The combination had activity that was superior to bortezomib monotherapy when dosed simultaneously or with Compound A first. Dosing of bortezomib prior to Compound A was not significantly different from either monotherapy.

### Example 13

Exposure of cells in vitro to Compound A followed by the proteasome inhibitor bortezomib 24 hours later resulted in increased cancer cell death compared to the reverse order of addition.

Combination studies with Compound A and bortezomib in nude mice were initiated, administering both drugs on q4d×3 schedule simultaneously and separated by 24 hours in both orders of addition. Studies to establish the maximum tolerated dose (MTD) of simultaneous and sequenced administration of Compound A and bortezomib identified a marked sequence dependence. Administration of bortezomib prior to Compound A was least well-tolerated, while on the reverse sequence both drugs could be administered at their respective single agent MTDs.

Anti-tumor activity of sequenced (24 hour offset, q4d×3) and simultaneous administration was explored in HT29 tumor xenografts, a tumor moderately sensitive to KSP inhibitors. Graphical results of the comparison of 10 mg/kg Compound A and 1.5 mg/kg bortezomib as single agents or in combination are shown in FIGS. 5A and 5B, respectively. The mean tumor growth delay (TGD) for 1.5 mg/kg bortezomib was 9 days, for 10 mg/kg Compound A was 24 days, and for the combination of bortezomib and Compound A was 33 days.

The second arm of the study compared the effects of sequence of administration for 1.5 mg/kg bortezomib and 10 mg/kg Compound A. There was a 12 day TGD for Compound A and a 9 day TGD for bortezomib alone. The combination of agents with Compound A administered first gave a 25 day TGD, simultaneous administration gave a 24 day TGD, and bortezomib administered first gave an 11 day TGD.

The mean time to endpoint (TTE), defined as tumor volume=1000 mm̂A3, of untreated control animals was 25.4 days. The mean TTE of bortezomib and Compound A as single agents at MTD did not differ significantly from untreated control animals. Administration of Compound A 24 hours prior to bortezomib resulted in a TTE of 58 days, which was superior to untreated control (p=0.004) or the best single agent (p=0.038) and comparable to activity of paclitaxel (TTE=59 days).

In summary, exposure of cells to the KSP inhibitor Compound A followed by the proteasome inhibitor bortezomib 24 hours later resulted in increased cancer cell death compared to the reverse order of addition. The combination had activity that was superior to bortezomib monotherapy when dosed simultaneously or with Compound A first. Dosing of bortezomib prior to Compound A was not significantly different from either monotherapy.

All publications, including but not limited to patents and patent applications, cited in this specification are herein incorporated by reference as if each individual publication were specifically and individually indicated to be incorporated by reference herein as though fully set forth.

It is to be understood that the present invention covers all combinations of groups described herein above. Particular examples or embodiments are non-limiting unless expressly described as such herein.

It will be apparent to those skilled in the art that various modifications may be made without departing from the spirit of the invention, such that the right is reserved to illustrated embodiments and all modifications coming within the scope of the following claims.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation the following claims.

1. A method for treating at least one hematologic cancer comprising administering to a patient
[a] an effective amount of a chromenone derivative chosen from compounds of Formula I 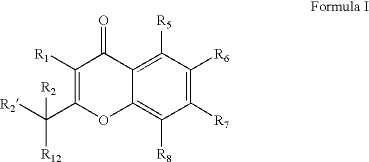
wherein:
R₁ is chosen from hydrogen, optionally substituted alkyl-, optionally substituted aryl-, optionally substituted aralkyl-, optionally substituted heteroaryl-, and optionally substituted heteroaralkyl-;
R₂ and R_{2′} are independently chosen from hydrogen, optionally substituted alkyl-, optionally substituted alkoxy, optionally substituted aryl-, optionally substituted aralkyl-, optionally substituted heteroaryl-, and optionally substituted heteroaralkyl-; or R₂ and R_{2′} taken together form an optionally substituted 3- to 7-membered ring;
R₁₂ is selected from the group consisting of optionally substituted imidazolyl, optionally substituted imidazolinyl, —NHR₄; —N(R₄)(COR₃); —N(R₄)(SO₂R₃ₐ); and —N(R₄)(CH₂R_{3b});
R₃ is chosen from hydrogen, optionally substituted alkyl-, optionally substituted aryl-, optionally substituted aralkyl-, optionally substituted heteroaryl-, optionally substituted heteroaralkyl-, R₁₅O— and R₁₇—NH—;
R₃ₐ is chosen from optionally substituted alkyl-, optionally substituted aryl-, optionally substituted aralkyl-, optionally substituted heteroaryl-, optionally substituted heteroaralkyl-, and R₁₇—NH—;
R_{3b} is chosen from hydrogen, optionally substituted alkyl-, optionally substituted aryl-, optionally substituted aralkyl-, optionally substituted heteroaryl-, and optionally substituted heteroaralkyl-;
R₄ is chosen from hydrogen, optionally substituted alkyl-, optionally substituted aryl-, optionally substituted aralkyl-, optionally substituted heterocyclyl-, and optionally substituted heteroaralkyl-;
R₅, R₆, R₇ and R₈ are independently chosen from hydrogen, acyl, optionally substituted alkyl-, optionally substituted alkoxy, halogen, hydroxyl, nitro, cyano, dialkylamino, alkylsulfonyl-, alkylsulfonamido-, alkylthio-, carboxyalkyl-, carboxamido-, aminocarbonyl-, optionally substituted aryl and optionally substituted heteroaryl-;
R₁₅ is chosen from optionally substituted alkyl-, optionally substituted aryl-, optionally substituted aralkyl-, optionally substituted heteroaryl-, and optionally substituted heteroaralkyl-; and
R₁₇ is hydrogen, optionally substituted alkyl-, optionally substituted aryl-, optionally substituted aralkyl-, optionally substituted heteroaryl-, or optionally substituted hetero-aralkyl, and
pharmaceutically acceptable salts thereof, and
[b] an effective amount of at least one prophylactic granulopoetic support.

2. The method of claim 1 wherein the at least one prophylactic granulopoetic support comprises granulocyte colony stimulating factor.

3. The method of claim 1 wherein the hematologic cancer is chosen from Hodgkin's Disease and Non-Hodgkin's Lymphoma.

4. A method of treating cancer comprising administering to a patient
[a] an effective amount of a chromenone derivative chosen from compounds of Formula I 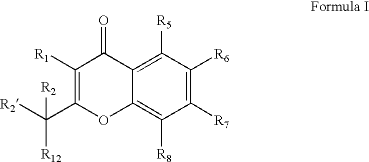
wherein:
R₁ is chosen from hydrogen, optionally substituted alkyl-, optionally substituted aryl-, optionally substituted aralkyl-, optionally substituted heteroaryl-, and optionally substituted heteroaralkyl-;
R₂ and R_{2′} are independently chosen from hydrogen, optionally substituted alkyl-, optionally substituted alkoxy, optionally substituted aryl-, optionally substituted aralkyl-, optionally substituted heteroaryl-, and optionally substituted heteroaralkyl-; or R₂ and R_{2′} taken together form an optionally substituted 3- to 7-membered ring;
R₁₂ is selected from the group consisting of optionally substituted imidazolyl, optionally substituted imidazolinyl, —NHR₄; —N(R₄)(COR₃); —N(R₄)(SO₂R₃ₐ); and —N(R₄)(CH₂R_{3b});
R₃ is chosen from hydrogen, optionally substituted alkyl-, optionally substituted aryl-, optionally substituted aralkyl-, optionally substituted heteroaryl-, optionally substituted heteroaralkyl-, R₁₅O— and R₁₇—NH—;
R₃ₐ is chosen from optionally substituted alkyl-, optionally substituted aryl-, optionally substituted aralkyl-, optionally substituted heteroaryl-, optionally substituted heteroaralkyl-, and R₁₇—NH—;
R_{3b} is chosen from hydrogen, optionally substituted alkyl-, optionally substituted aryl-, optionally substituted aralkyl-, optionally substituted heteroaryl-, and optionally substituted heteroaralkyl-;
R₄ is chosen from hydrogen, optionally substituted alkyl-, optionally substituted aryl-, optionally substituted aralkyl-, optionally substituted heterocyclyl-, and optionally substituted heteroaralkyl-;
R₅, R₆, R₇ and R₈ are independently chosen from hydrogen, acyl, optionally substituted alkyl-, optionally substituted alkoxy, halogen, hydroxyl, nitro, cyano, dialkylamino, alkylsulfonyl-, alkylsulfonamido-, alkylthio-, carboxyalkyl-, carboxamido-, aminocarbonyl-, optionally substituted aryl and optionally substituted heteroaryl-;
R₁₅ is chosen from optionally substituted alkyl-, optionally substituted aryl-, optionally substituted aralkyl-, optionally substituted heteroaryl-, and optionally substituted heteroaralkyl-; and
R₁₇ is hydrogen, optionally substituted alkyl-, optionally substituted aryl-, optionally substituted aralkyl-, optionally substituted heteroaryl-, or optionally substituted hetero-aralkyl, and
pharmaceutically acceptable salts thereof, and
[b] an effective amount of at least one proteasome inhibitor, wherein the proteasome inhibitor is administered after the chromenone derivative.

5. The method of claim 4 wherein the proteasome inhibitor is bortezomib.

6. The method of claim 4 wherein the proteasome inhibitor is administered at least 12 hours after administration of the chromenone derivative.

7. The method of claim 6 wherein the proteasome inhibitor is administered about 24 hours after administration of the chromenone derivative.

8. A method for treating cancer comprising administering to a patient
[a] an effective amount of a chromenone derivative chosen from compounds of Formula I 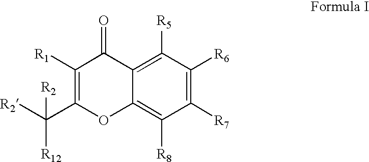
wherein:
R₁ is chosen from hydrogen, optionally substituted alkyl-, optionally substituted aryl-, optionally substituted aralkyl-, optionally substituted heteroaryl-, and optionally substituted heteroaralkyl-;
R₂ and R_{2′} are independently chosen from hydrogen, optionally substituted alkyl-, optionally substituted alkoxy, optionally substituted aryl-, optionally substituted aralkyl-, optionally substituted heteroaryl-, and optionally substituted heteroaralkyl-; or R₂ and R_{2′} taken together form an optionally substituted 3- to 7-membered ring;
R₁₂ is selected from the group consisting of optionally substituted imidazolyl, optionally substituted imidazolinyl, —NHR₄; —N(R₄)(COR₃); —N(R₄)(SO₂R₃ₐ); and —N(R₄)(CH₂R_{3b});
R₃ is chosen from hydrogen, optionally substituted alkyl-, optionally substituted aryl-, optionally substituted aralkyl-, optionally substituted heteroaryl-, optionally substituted heteroaralkyl-, R₁₅O— and R₁₇—NH—;
R₃ₐ is chosen from optionally substituted alkyl-, optionally substituted aryl-, optionally substituted aralkyl-, optionally substituted heteroaryl-, optionally substituted heteroaralkyl-, and R₁₇—NH—;
R_{3b} is chosen from hydrogen, optionally substituted alkyl-, optionally substituted aryl-, optionally substituted aralkyl-, optionally substituted heteroaryl-, and optionally substituted heteroaralkyl-;
R₄ is chosen from hydrogen, optionally substituted alkyl-, optionally substituted aryl-, optionally substituted aralkyl-, optionally substituted heterocyclyl-, and optionally substituted heteroaralkyl-;
R₅, R₆, R₇ and R₈ are independently chosen from hydrogen, acyl, optionally substituted alkyl-, optionally substituted alkoxy, halogen, hydroxyl, nitro, cyano, dialkylamino, alkylsulfonyl-, alkylsulfonamido-, alkylthio-, carboxyalkyl-, carboxamido-, aminocarbonyl-, optionally substituted aryl and optionally substituted heteroaryl-;
R₁₅ is chosen from optionally substituted alkyl-, optionally substituted aryl-, optionally substituted aralkyl-, optionally substituted heteroaryl-, and optionally substituted heteroaralkyl-; and
R₁₇ is hydrogen, optionally substituted alkyl-, optionally substituted aryl-, optionally substituted aralkyl-, optionally substituted heteroaryl-, or optionally substituted hetero-aralkyl, and
pharmaceutically acceptable salts thereof, and
[b] an effective amount of at least one chemotherapeutic agent selected from neutropenia treatment agents, alkylating agents, antimetabolites, platinating agents; topoisomerase inhibitors, tubulin agents, signalling inhibitors, proteasome inhibitors, and other chemotherapeutic agents.

9. The method of claim 1 wherein R₁ is selected from hydrogen, optionally substituted C₁-C₈ alkyl, optionally substituted aryl, optionally substituted heteroaryl, optionally substituted aryl-C₁-C₄-alkyl-, and optionally substituted heteroaryl-C₁-C₄-alkyl-.

10. The method of claim 9 wherein R₁ is optionally substituted phenyl-C₁-C₄-alkyl- or optionally substituted heteroaryl-C₁-C₄-alkyl-.

11. The method of claim 10 wherein R₁ is naphthyl, phenyl, bromophenyl, chlorophenyl, methoxyphenyl, ethoxyphenyl, tolyl, dimethylphenyl, chorofluorophenyl, methylchlorophenyl, ethylphenyl, phenethyl, benzyl, chlorobenzyl, methylbenzyl, methoxybenzyl, cyanobenzyl, hydroxybenzyl, dichlorobenzyl, dimethoxybenzyl, or naphthalenylmethyl.

12. The method of claim 11 wherein R₁ is benzyl, cyanobenzyl, methoxybenzyl, or naphthalenylmethyl.

13. The method of claim 12 wherein R₁ is benzyl.

14. The method of claim 1 wherein R₂ is optionally substituted C₁-C₄ alkyl, and R_{2′} is hydrogen or optionally substituted C₁-C₄ alkyl.

15. The method of claim 14 wherein R_{2′} is hydrogen and R₂ is optionally substituted C₁-C₄ alkyl.

16. The method of claim 15 wherein R₂ is chosen from methyl, ethyl, propyl, butyl, methylthioethyl, methylthiomethyl, aminobutyl, (CBZ)aminobutyl, cyclohexylmethyl, benzyloxymethyl, methylsulfinylethyl, methylsulfinylmethyl, and hydroxymethyl, and R_{2′} is hydrogen.

17. The method of claim 16 wherein R_{2′} is hydrogen and R₂ is ethyl or propyl.

18. The method of claim 17 wherein R₂ is i-propyl.

19. The method of claim 1 wherein R₃ is selected from optionally substituted C₁-C₈ alkyl, optionally substituted aryl-C₁-C₄-alkyl-, optionally substituted heteroaryl-C₁-C₄-alkyl-, optionally substituted heteroaryl, optionally substituted aryl, R₁₅O— and R₁₇—NH—, R₁₅ is chosen from optionally substituted C₁-C₈ alkyl and optionally substituted aryl, and R₁₇ is chosen from hydrogen, optionally substituted C₁-C₈ alkyl and optionally substituted aryl.

20. The method of claim 19 wherein R₃ is chosen from optionally substituted C₁-C₈ alkyl, optionally substituted heteroaryl, and optionally substituted aryl.

21. The method of claim 20 wherein R₃ is tolyl, halophenyl, halomethylphenyl, hydroxymethylphenyl, methylenedioxyphenyl, formylphenyl or cyanophenyl.

22. The method of claim 1 wherein R₄ is R₁₆-alkylene-, wherein R₁₆ is amino, C₁-C₄ alkylamino-, di(C₁-C₄ alkyl)amino-, C₁-C₄ alkoxy-, hydroxyl, or N-heterocyclyl.

23. The method of claim 22 wherein R₁₆ is amino.

24. The method of claim 22 wherein R₄ is aminoethyl, aminopropyl, aminobutyl, aminopentyl, aminohexyl, methylaminoethyl, methylaminopropyl, methylaminobutyl, methylaminopentyl, methylaminohexyl, dimethylaminoethyl, dimethylaminopropyl, dimethylaminobutyl, dimethylaminopentyl, dimethylaminohexyl, ethylaminoethyl, ethylaminopropyl, ethylaminobutyl, ethylaminopentyl, ethylaminohexyl, diethylaminoethyl, diethylaminopropyl, diethylaminobutyl, diethylaminopentyl, or diethylaminohexyl.

25. The method of claim 1 wherein R₅, R₆, R₇, and R₈ are independently chosen from hydrogen, amino, alkylamino, hydroxyl, halogen, C₁-C₄ alkyl, C₁-C₄ haloalkyl, C₁-C₄ alkoxy, C₁-C₄ haloalkoxy and cyano.

26. The method of claim 1 wherein the compound of Formula I is chosen from:
N-(3-Amino-propyl)-N-{1-[3-(3-cyano-benzyl)-7-hydroxy-4-oxo-4H-chromen-2-yl]-2-methyl-propyl}-3-fluoro-4-methyl-benzamide;
4-Acetyl-N-(3-amino-propyl)-N-[1-(3-benzyl-7-methoxy-4-oxo-4H-chromen-2-yl)-2-methyl-propyl]-benzamide;
2-{1-[4-(2-Amino-ethyl)-2-p-tolyl-imidazol-1-yl]-2-methyl-propyl}-3-benzyl-4-oxo-4H-chromene-7-carbonitrile;
3-Benzyl-2-[1-(4,4-dimethyl-2-p-tolyl-4,5-dihydro-imidazol-1-yl)-2-methyl-propyl]-4-oxo-4H-chromene-7-carbonitrile;
Benzo[1,3]dioxole-5-carboxylic acid (3-amino-propyl)-{1-[7-chloro-3-(3-cyano-benzyl)-4-oxo-4H-chromen-2-yl]-2-methyl-propyl}-amide;
N-(3-Amino-propyl)-N-{1-[7-chloro-3-(3-cyano-benzyl)-4-oxo-4H-chromen-2-yl]-2-methyl-propyl}-4-methyl-benzamide;
N-(3-Amino-propyl)-N-[1-(7-chloro-3-naphthalen-1-ylmethyl-4-oxo-4H-chromen-2-yl)-2-methyl-propyl]-2-methoxy-acetamide;
4-Acetyl-N-(3-amino-propyl)-N-[1-(3-benzyl-7-cyano-4-oxo-4H-chromen-2-yl)-2-methyl-propyl]-benzamide;
N-(3-Amino-propyl)-N-{1-[7-chloro-3-(3-cyano-benzyl)-4-oxo-4H-chromen-2-yl]-2-methyl-propyl}-3-fluoro-4-methyl-benzamide;
Benzo[1,3]dioxole-5-carboxylic acid (3-amino-propyl)-[1-(3-benzyl-7-cyano-4-oxo-4H-chromen-2-yl)-2-methyl-propyl]-amide;
Benzo[1,3]dioxole-5-carboxylic acid (3-amino-propyl)-[1-(3-benzyl-7-fluoro-4-oxo-4H-chromen-2-yl)-2-methyl-propyl]-amide;
3-Benzyl-2-[1-(4,4-dimethyl-2-p-tolyl-4,5-dihydro-imidazol-1-yl)-2-methyl-propyl]-7-hydroxy-chromen-4-one;
N-(3-Amino-propyl)-N-[1-(3-benzyl-7-fluoro-4-oxo-4H-chromen-2-yl)-2-methyl-propyl]-3-fluoro-4-methyl-benzamide;
N-(3-Amino-propyl)-N-[1-(3-benzyl-7-methoxy-4-oxo-4H-chromen-2-yl)-2-methyl-propyl]-3-fluoro-4-methyl-benzamide;
N-(3-Amino-propyl)-N-[1-(3-benzyl-7-methoxy-4-oxo-4H-chromen-2-yl)-2-methyl-propyl]-4-methyl-benzamide;
3-Benzyl-2-[1-(4,4-dimethyl-2-p-tolyl-4,5-dihydro-imidazol-1-yl)-2-methyl-propyl]-7-methoxy-chromen-4-one;
3-Benzyl-7-fluoro-2-{1-[2-(3-fluoro-4-methyl-phenyl)-4,4-dimethyl-4,5-dihydro-imidazol-1-yl]-2-methyl-propyl}-chromen-4-one;
3-Benzyl-2-[1-(4,4-dimethyl-2-p-tolyl-4,5-dihydro-imidazol-1-yl)-2-methyl-propyl]-7-fluoro-chromen-4-one;
3-Benzyl-2-{1-[2-(3-fluoro-4-methyl-phenyl)-4,4-dimethyl-4,5-dihydro-imidazol-1-yl]-2-methyl-propyl}-7-cyano-chromen-4-one;
N-(3-Amino-propyl)-N-[1-(3-benzyl-7-fluoro-4-oxo-4H-chromen-2-yl)-2-methyl-propyl]-4-methyl-benzamide;
4-Acetyl-N-(3-amino-propyl)-N-[1-(3-benzyl-7-chloro-4-oxo-4H-chromen-2-yl)-2-methyl-propyl]-benzamide;
N-(3-Amino-propyl)-3-fluoro-N-{1-[7-fluoro-3-(3-methoxy-benzyl)-4-oxo-4H-chromen-2-yl]-2-methyl-propyl}-4-methyl-benzamide;
4-Acetyl-N-(3-amino-propyl)-N-{1-[7-chloro-3-(3-cyano-benzyl)-4-oxo-4H-chromen-2-yl]-2-methyl-propyl}-benzamide;
(2-{1-[1-(3-Benzyl-7-chloro-4-oxo-4H-chromen-2-yl)-2-methyl-propyl]-2-p-tolyl-1H-imidazol-4-yl}-ethyl)-carbamic acid benzyl ester;
2-[1-(2-Benzo[1,3]dioxol-5-yl-4,4-dimethyl-4,5-dihydro-imidazol-1-yl)-2-methyl-propyl]-3-benzyl-7-cyano-chromen-4-one;
4-Acetyl-N-(3-amino-propyl)-N-[1-(3-benzyl-7-fluoro-4-oxo-4H-chromen-2-yl)-2-methyl-propyl]-benzamide;
N-(3-Amino-propyl)-N-[1-(3-benzyl-7-chloro-4-oxo-4H-chromen-2-yl)-2-methyl-propyl]-3-fluoro-4-methyl-benzamide;
N-(3-Amino-propyl)-N-{1-[7-fluoro-3-(3-methoxy-benzyl)-4-oxo-4H-chromen-2-yl]-2-methyl-propyl}-4-methoxy-benzamide;
Benzo[1,3]dioxole-5-carboxylic acid (3-amino-propyl)-{1-[7-fluoro-3-(3-methoxy-benzyl)-4-oxo-4H-chromen-2-yl]-2-methyl-propyl}-amide;
2-(1-[4-(2-Amino-ethyl)-2-p-tolyl-imidazol-1-yl]-2-methyl-propyl)-3-benzyl-7-chloro-chromen-4-one;
N-(3-Amino-propyl)-N-[1-(3-benzyl-7-hydroxy-4-oxo-4H-chromen-2-yl)-2-methyl-propyl]-3-fluoro-4-methyl-benzamide;
Benzo[1,3]dioxole-5-carboxylic acid (3-amino-propyl)-[1-(3-benzyl-7-chloro-4-oxo-4H-chromen-2-yl)-2-methyl-propyl]-amide;
N-(3-Amino-propyl)-N-[1-(3-benzyl-7-hydroxy-4-oxo-4H-chromen-2-yl)-2-methyl-propyl]-4-methyl-benzamide;
N-(3-Amino-propyl)-N-{1-[7-fluoro-3-(3-methoxy-benzyl)-4-oxo-4H-chromen-2-yl]-2-methyl-propyl}-2-methoxy-acetamide;
N-(3-Amino-propyl)-N-{1-[7-fluoro-3-(3-methoxy-benzyl)-4-oxo-4H-chromen-2-yl]-2-methyl-propyl}-4-methyl-benzamide;
N-(3-Amino-propyl)-N-[(3-benzyl-7-chloro-4-oxo-4H-chromen-2-yl)-cyclopropyl-methyl]-4-methyl-benzamide;
2,3-Dihydro-benzo[1,4]dioxine-6-carboxylic acid (3-amino-propyl)-[1-(3-benzyl-7-chloro-4-oxo-4H-chromen-2-yl)-2-methyl-propyl]-amide;
N-(3-Amino-propyl)-3-fluoro-N-{1-[7-cyano-3-(3-methoxy-benzyl)-4-oxo-4H-chromen-2-yl]-2-methyl-propyl}-4-methyl-benzamide;
3-Benzyl-7-chloro-2-[2-methyl-1-(2-p-tolyl-4,5-dihydro-imidazol-1-yl)-propyl]-chromen-4-one;
3-Benzyl-7-fluoro-2-{1-[2-(3-fluoro-4-methyl-phenyl)-4,5-dihydro-imidazol-1-yl]-2-methyl-propyl}-chromen-4-one;
2-[1-(4-Aminomethyl-2-p-tolyl-imidazol-1-yl)-2-methyl-propyl]-3-benzyl-7-chloro-chromen-4-one;
3-Benzyl-7-methoxy-2-[2-methyl-1-(2-p-tolyl-4,5-dihydro-imidazol-1-yl)-propyl]-chromen-4-one;
2-[1-(2-Benzo[1,3]dioxol-5-yl-4,5-dihydro-imidazol-1-yl)-2-methyl-propyl]-3-benzyl-7-chloro-chromen-4-one;
4-Acetyl-N-(3-amino-propyl)-N-[1-(3-benzyl-7-chloro-4-oxo-4H-chromen-2-yl)-2-methyl-propyl]-benzamide;
4-Acetyl-N-(3-amino-propyl)-N-{1-[7-cyano-3-(3-methoxy-benzyl)-4-oxo-4H-chromen-2-yl]-2-methyl-propyl}-benzamide;
3-Benzyl-7-chloro-2-{1-[2-(3-fluoro-4-methyl-phenyl)-4,5-dihydro-imidazol-1-yl]-2-methyl-propyl}-chromen-4-one;
3-Benzyl-7-fluoro-2-[2-methyl-1-(2-p-tolyl-4,5-dihydro-imidazol-1-yl)-propyl]-chromen-4-one;
2-[1-(2-Benzo[1,3]dioxol-5-yl-4,5-dihydro-imidazol-1-yl)-2-methyl-propyl]-3-benzyl-7-fluoro-chromen-4-one;
N-(3-Amino-propyl)-N-{1-[7-cyano-3-(3-methoxy-benzyl)-4-oxo-4H-chromen-2-yl]-2-methyl-propyl}-4-methyl-benzamide;
Benzo[1,3]dioxole-5-carboxylic acid (3-amino-propyl)-{1-[7-cyano-3-(3-methoxy-benzyl)-4-oxo-4H-chromen-2-yl]-2-methyl-propyl}-amide;
3-(2-{1-[2-(3-Fluoro-4-methyl-phenyl)-4,5-dihydro-imidazol-1-yl]-2-methyl-propyl}-7-methoxy-4-oxo-4H-chromen-3-ylmethyl)-benzonitrile;
3-{7-Chloro-2-[2-methyl-1-(2-p-tolyl-4,5-dihydro-imidazol-1-yl)-propyl]-4-oxo-4H-chromen-3-ylmethyl}-benzonitrile;
3-{7-Methoxy-2-[2-methyl-1-(2-p-tolyl-4,5-dihydro-imidazol-1-yl)-propyl]-4-oxo-4H-chromen-3-ylmethyl}-benzonitrile;
3-{7-Fluoro-2-[2-methyl-1-(2-p-tolyl-4,5-dihydro-imidazol-1-yl)-propyl]-4-oxo-4H-chromen-3-ylmethyl}-benzonitrile;
N-(3-Amino-propyl)-N-{1-[7-chloro-3-(3-methoxy-benzyl)-4-oxo-4H-chromen-2-yl]-2-methyl-propyl}-3-fluoro-4-methyl-benzamide;
N-(3-Amino-propyl)-N-{1-[3-(3-cyano-benzyl)-7-methoxy-4-oxo-4H-chromen-2-yl]-2-methyl-propyl}-4-methyl-benzamide;
N-(3-Amino-propyl)-N-{1-[3-(3-cyano-benzyl)-7-methoxy-4-oxo-4H-chromen-2-yl]-2-methyl-propyl}-3-fluoro-4-methyl-benzamide;
3-{2-[1-(2-Benzo[1,3]dioxol-5-yl-4,5-dihydro-imidazol-1-yl)-2-methyl-propyl]-7-fluoro-4-oxo-4H-chromen-3-ylmethyl}-benzonitrile;
3-Benzyl-2-{1-[2-(3-fluoro-4-methyl-phenyl)-4,5-dihydro-imidazol-1-yl]-2-methyl-propyl}-4-oxo-4H-chromene-7-carbonitrile;
2-[1-(2-Benzo[1,3]dioxol-5-yl-4,5-dihydro-imidazol-1-yl)-2-methyl-propyl]-3-benzyl-4-oxo-4H-chromene-7-carbonitrile;
3-(7-Chloro-2-{1-[2-(3-fluoro-4-methyl-phenyl)-4,5-dihydro-imidazol-1-yl]-2-methyl-propyl}-4-oxo-4H-chromen-3-ylmethyl)-benzonitrile;
3-{2-[1-(2-Benzo[1,3]dioxol-5-yl-4,5-dihydro-imidazol-1-yl)-2-methyl-propyl]-7-methoxy-4-oxo-4H-chromen-3-ylmethyl}-benzonitrile;
3-(7-Fluoro-2-{1-[2-(3-fluoro-4-methyl-phenyl)-4,5-dihydro-imidazol-1-yl]-2-methyl-propyl}-4-oxo-4H-chromen-3-ylmethyl)-benzonitrile;
Benzo[1,3]dioxole-5-carboxylic acid (3-amino-propyl)-{1-[7-cyano-3-(3-cyano-benzyl)-4-oxo-4H-chromen-2-yl]-2-methyl-propyl}-amide;
Benzo[1,3]dioxole-5-carboxylic acid (3-amino-propyl)-{1-[3-(3-cyano-benzyl)-7-methoxy-4-oxo-4H-chromen-2-yl]-2-methyl-propyl}-amide;
Benzo[1,3]dioxole-5-carboxylic acid (3-amino-propyl)-{1-[3-(3-cyano-benzyl)-7-fluoro-4-oxo-4H-chromen-2-yl]-2-methyl-propyl}-amide;
N-(3-Amino-propyl)-N-{1-[3-(3-cyano-benzyl)-7-fluoro-4-oxo-4H-chromen-2-yl]-2-methyl-propyl}-4-methyl-benzamide;
Benzo[1,3]dioxole-5-carboxylic acid (3-amino-propyl)-{1-[7-chloro-3-(3-methoxy-benzyl)-4-oxo-4H-chromen-2-yl]-2-methyl-propyl}-amide;
N-(3-Amino-propyl)-N-{1-[7-cyano-3-(3-cyano-benzyl)-4-oxo-4H-chromen-2-yl]-2-methyl-propyl}-4-methyl-benzamide;
3-Benzyl-2-[2-methyl-1-(2-p-tolyl-4,5-dihydro-imidazol-1-yl)-propyl]-4-oxo-4H-chromene-7-carbonitrile;
2-[1-(2-Benzo[1,3]dioxol-5-yl-4,5-dihydro-imidazol-1-yl)-2-methyl-propyl]-3-benzyl-7-methoxy-chromen-4-one;
N-(3-Amino-propyl)-N-{1-[7-chloro-3-(3-methoxy-benzyl)-4-oxo-4H-chromen-2-yl]-2-methyl-propyl}-2-methoxy-acetamide;
N-(3-Amino-propyl)-N-{1-[7-cyano-3-(3-cyano-benzyl)-4-oxo-4H-chromen-2-yl]-2-methyl-propyl}-3-fluoro-4-methyl-benzamide;
N-(3-Amino-propyl)-N-{1-[3-(3-cyano-benzyl)-7-fluoro-4-oxo-4H-chromen-2-yl]-2-methyl-propyl}-3-fluoro-4-methyl-benzamide;
2-{1-[4-(2-Amino-ethyl)-2-p-tolyl-imidazol-1-yl]-2-methyl-propyl}-7-chloro-3-(3-methoxy-benzyl)-chromen-4-one;
2-{1-[4-(2-Amino-ethyl)-2-(3-fluoro-4-methyl-phenyl)-imidazol-1-yl]-2-methyl-propyl}-3-benzyl-7-fluoro-chromen-4-one;
2-{1-[4-(2-Amino-ethyl)-2-(3-fluoro-4-methyl-phenyl)-imidazol-1-yl]-2-methyl-propyl}-3-benzyl-4-oxo-4H-chromene-7-carbonitrile;
2-{1-[4-(2-Amino-ethyl)-2-p-tolyl-imidazol-1-yl]-2-methyl-propyl}-3-benzyl-7-fluoro-chromen-4-one;
2-{1-[4-(2-Amino-ethyl)-2-(3-fluoro-4-methyl-phenyl)-imidazol-1-yl]-2-methyl-propyl}-7-chloro-3-(3-methoxy-benzyl)-chromen-4-one;
2-{1-[4-(2-Amino-ethyl)-2-(3-fluoro-4-methyl-phenyl)-imidazol-1-yl]-2-methyl-propyl}-3-benzyl-7-chloro-chromen-4-one;
3-Benzyl-2-{1-[2-(3-fluoro-4-methyl-phenyl)-4,5-dihydro-imidazol-1-yl]-2-methyl-propyl}-7-methoxy-chromen-4-one;
3-(2-{1-[4-(2-Amino-ethyl)-2-p-tolyl-imidazol-1-yl]-2-methyl-propyl}-7-chloro-4-oxo-4H-chromen-3-ylmethyl)-benzonitrile;
3-(2-{1-[4-(2-Amino-ethyl)-2-(3-fluoro-4-methyl-phenyl)-imidazol-1-yl]-2-methyl-propyl}-7-chloro-4-oxo-4H-chromen-3-ylmethyl)-benzonitrile;
2-{1-[4-(2-Amino-ethyl)-2-p-tolyl-imidazol-1-yl]-2-methyl-propyl}-7-fluoro-3-(3-methoxy-benzyl)-chromen-4-one;
2-{1-[4-(2-Amino-ethyl)-2-(3-fluoro-4-methyl-phenyl)-imidazol-1-yl]-2-methyl-propyl}-3-(3-cyano-benzyl)-4-oxo-4H-chromene-7-carbonitrile;
N-(3-Amino-propyl)-3-fluoro-N-{1-[7-hydroxy-3-(3-methoxy-benzyl)-4-oxo-4H-chromen-2-yl]-2-methyl-propyl}-4-methyl-benzamide;
2-{1-[4-(2-Amino-ethyl)-2-p-tolyl-imidazol-1-yl]-2-methyl-propyl}-3-(3-methoxy-benzyl)-4-oxo-4H-chromene-7-carbonitrile;
N-(3-Amino-propyl)-N-[1-(3-benzyl-7-cyano-4-oxo-4H-chromen-2-yl)-2-methyl-propyl]-4-ethoxy-benzamide;
N-(3-Amino-propyl)-N-[1-(3-benzyl-7-cyano-4-oxo-4H-chromen-2-yl)-2-methyl-propyl]-6-trifluoromethyl-nicotinamide;
N-(3-Amino-propyl)-N-[1-(3-benzyl-7-fluoro-4-oxo-4H-chromen-2-yl)-2-methyl-propyl]-6-trifluoromethyl-nicotinamide;
N-(3-Amino-propyl)-N-[1-(3-benzyl-7-fluoro-4-oxo-4H-chromen-2-yl)-2-methyl-propyl]-isonicotinamide;
N-(3-Amino-propyl)-N-[1-(3-benzyl-7-chloro-4-oxo-4H-chromen-2-yl)-2-methyl-propyl]-4-cyano-benzamide;
4-Acetylamino-N-(3-amino-propyl)-N-[1-(3-benzyl-7-chloro-4-oxo-4H-chromen-2-yl)-2-methyl-propyl]-benzamide;
N-(3-Amino-propyl)-N-[1-(3-benzyl-7-chloro-4-oxo-4H-chromen-2-yl)-2-methyl-propyl]-6-trifluoromethyl-nicotinamide;
Benzo[1,2,3]thiadiazole-5-carboxylic acid (3-amino-propyl)-[1-(3-benzyl-7-chloro-4-oxo-4H-chromen-2-yl)-2-methyl-propyl]-amide;
Benzo[1,2,3]thiadiazole-5-carboxylic acid (3-amino-propyl)-[1-(3-benzyl-7-fluoro-4-oxo-4H-chromen-2-yl)-2-methyl-propyl]-amide;
4-Acetylamino-N-(3-amino-propyl)-N-[1-(3-benzyl-7-fluoro-4-oxo-4H-chromen-2-yl)-2-methyl-propyl]-benzamide;
Benzo[1,2,3]thiadiazole-5-carboxylic acid (3-amino-propyl)-[1-(3-benzyl-7-fluoro-4-oxo-4H-chromen-2-yl)-2-methyl-propyl]-amide;
N-(3-Amino-propyl)-N-[1-(3-benzyl-7-chloro-4-oxo-4H-chromen-2-yl)-2-methyl-propyl]-nicotinamide;
N-(3-Amino-propyl)-N-[1-(3-benzyl-7-fluoro-4-oxo-4H-chromen-2-yl)-2-methyl-propyl]-4-methoxy-benzamide;
Benzo[1,2,3]thiadiazole-5-carboxylic acid (3-amino-propyl)-[1-(3-benzyl-7-cyano-4-oxo-4H-chromen-2-yl)-2-methyl-propyl]-amide;
5-Methyl-pyrazine-2-carboxylic acid (3-amino-propyl)-[1-(3-benzyl-7-chloro-4-oxo-4H-chromen-2-yl)-2-methyl-propyl]-amide;
N-(3-Amino-propyl)-N-[1-(3-benzyl-7-cyano-4-oxo-4H-chromen-2-yl)-2-methyl-propyl]-3-dimethylamino-benzamide;
2-[1-(2-Benzo[1,3]dioxol-5-yl-4,5-dihydro-imidazol-1-yl)-2-methyl-propyl]-3-(3-methoxy-benzyl)-4-oxo-4H-chromene-7-carbonitrile;
7-Chloro-3-(3-methoxy-benzyl)-2-[2-methyl-1-(2-p-tolyl-4,5-dihydro-imidazol-1-yl)-propyl]-chromen-4-one;
7-Chloro-2-{1-[2-(3-fluoro-4-methyl-phenyl)-4,5-dihydro-imidazol-1-yl]-2-methyl-propyl}-3-(3-methoxy-benzyl)-chromen-4-one;
2-[1-(2-Benzo[1,3]dioxol-5-yl-4,5-dihydro-imidazol-1-yl)-2-methyl-propyl]-7-chloro-3-(3-methoxy-benzyl)-chromen-4-one;
7-Fluoro-3-(3-methoxy-benzyl)-2-[2-methyl-1-(2-p-tolyl-4,5-dihydro-imidazol-1-yl)-propyl]-chromen-4-one;
7-Fluoro-2-{1-[2-(3-fluoro-4-methyl-phenyl)-4,5-dihydro-imidazol-1-yl]-2-methyl-propyl}-3-(3-methoxy-benzyl)-chromen-4-one;
2-{1-[4-(2-Amino-ethyl)-2-(3-fluoro-4-methyl-phenyl)-imidazol-1-yl]-2-methyl-propyl}-7-fluoro-3-(3-methoxy-benzyl)-chromen-4-one;
2-{1-[4-(2-Acetylamino-ethyl)-2-(3-fluoro-4-methyl-phenyl)-imidazol-1-yl]-2-methyl-propyl}-3-(3-methoxy-benzyl)-4-oxo-4H-chromene-7-carboxylic acid amide;
3-(2-{1-[4-(2-Amino-ethyl)-2-(3-fluoro-4-methyl-phenyl)-imidazol-1-yl]-2-methyl-propyl}-7-fluoro-4-oxo-4H-chromen-3-ylmethyl)-benzonitrile;
N-{1-[1-(3-Benzyl-7-chloro-4-oxo-4H-chromen-2-yl)-2-methyl-propyl]-2-p-tolyl-1H-imidazol-4-ylmethyl}-acetamide;
Benzo[b]thiophene-2-carboxylic acid (3-amino-propyl)-[1-(3-benzyl-7-cyano-4-oxo-4H-chromen-2-yl)-2-methyl-propyl]-amide;
1-Methyl-1H-indole-2-carboxylic acid (3-amino-propyl)-[1-(3-benzyl-7-cyano-4-oxo-4H-chromen-2-yl)-2-methyl-propyl]-amide;
5-tert-Butyl-2-methyl-2H-pyrazole-3-carboxylic acid (3-amino-propyl)-[1-(3-benzyl-7-cyano-4-oxo-4H-chromen-2-yl)-2-methyl-propyl]-amide;
2,5-Dimethyl-2H-pyrazole-3-carboxylic acid (3-amino-propyl)-[1-(3-benzyl-7-cyano-4-oxo-4H-chromen-2-yl)-2-methyl-propyl]-amide;
5-tert-Butyl-2-methyl-2H-pyrazole-3-carboxylic acid (3-amino-propyl)-[1-(3-benzyl-7-fluoro-4-oxo-4H-chromen-2-yl)-2-methyl-propyl]-amide;
2,5-Dimethyl-2H-pyrazole-3-carboxylic acid (3-amino-propyl)-[1-(3-benzyl-7-chloro-4-oxo-4H-chromen-2-yl)-2-methyl-propyl]-amide;
2,5-Dimethyl-2H-pyrazole-3-carboxylic acid (3-amino-propyl)-[1-(3-benzyl-7-fluoro-4-oxo-4H-chromen-2-yl)-2-methyl-propyl]-amide;
5-Methyl-2H-pyrazole-3-carboxylic acid (3-amino-propyl)-[1-(3-benzyl-7-cyano-4-oxo-4H-chromen-2-yl)-2-methyl-propyl]-amide;
5-Methyl-2H-pyrazole-3-carboxylic acid (3-amino-propyl)-[1-(3-benzyl-7-fluoro-4-oxo-4H-chromen-2-yl)-2-methyl-propyl]-amide;
Furan-2-carboxylic acid (3-amino-propyl)-[1-(3-benzyl-7-fluoro-4-oxo-4H-chromen-2-yl)-2-methyl-propyl]-amide;
Furan-2-carboxylic acid (3-amino-propyl)-[1-(3-benzyl-7-chloro-4-oxo-4H-chromen-2-yl)-2-methyl-propyl]-amide;
2,5-Dimethyl-furan-3-carboxylic acid (3-amino-propyl)-[1-(3-benzyl-7-cyano-4-oxo-4H-chromen-2-yl)-2-methyl-propyl]-amide;
2,5-Dimethyl-furan-3-carboxylic acid (3-amino-propyl)-[1-(3-benzyl-7-fluoro-4-oxo-4H-chromen-2-yl)-2-methyl-propyl]-amide;
5-Methyl-thiophene-2-carboxylic acid (3-amino-propyl)-[1-(3-benzyl-7-cyano-4-oxo-4H-chromen-2-yl)-2-methyl-propyl]-amide;
5-Methyl-thiophene-2-carboxylic acid (3-amino-propyl)-[1-(3-benzyl-7-fluoro-4-oxo-4H-chromen-2-yl)-2-methyl-propyl]-amide;
5-Methyl-isoxazole-3-carboxylic acid (3-amino-propyl)-[1-(3-benzyl-7-chloro-4-oxo-4H-chromen-2-yl)-2-methyl-propyl]-amide;
5-Methyl-2-trifluoromethyl-furan-3-carboxylic acid (3-amino-propyl)-[1-(3-benzyl-7-cyano-4-oxo-4H-chromen-2-yl)-2-methyl-propyl]-amide;
5-Methyl-isoxazole-3-carboxylic acid (3-amino-propyl)-[1-(3-benzyl-7-fluoro-4-oxo-4H-chromen-2-yl)-2-methyl-propyl]-amide;
5-Methyl-isoxazole-3-carboxylic acid (3-amino-propyl)-[1-(3-benzyl-7-cyano-4-oxo-4H-chromen-2-yl)-2-methyl-propyl]-amide;
Benzo[c]isoxazole-3-carboxylic acid (3-amino-propyl)-[1-(3-benzyl-7-chloro-4-oxo-4H-chromen-2-yl)-2-methyl-propyl]-amide;
Benzo[c]isoxazole-3-carboxylic acid (3-amino-propyl)-[1-(3-benzyl-7-fluoro-4-oxo-4H-chromen-2-yl)-2-methyl-propyl]-amide;
1-Methyl-1H-pyrrole-2-carboxylic acid (3-amino-propyl)-[1-(3-benzyl-7-fluoro-4-oxo-4H-chromen-2-yl)-2-methyl-propyl]-amide;
1-Methyl-1H-imidazole-4-carboxylic acid (3-amino-propyl)-[(R)-1-(3-benzyl-7-cyano-4-oxo-4H-chromen-2-yl)-2-methyl-propyl]-amide;
N-(3-Amino-propyl)-N—[(R)-1-(3-benzyl-7-cyano-4-oxo-4H-chromen-2-yl)-2-methyl-propyl]-3-dimethylamino-benzamide;
5-Methyl-2-trifluoromethyl-furan-3-carboxylic acid (3-amino-propyl)-[(R)-1-(3-benzyl-7-cyano-4-oxo-4H-chromen-2-yl)-2-methyl-propyl]-amide;
5-Methyl-isoxazole-3-carboxylic acid (3-amino-propyl)-[(R)-1-(3-benzyl-7-cyano-4-oxo-4H-chromen-2-yl)-2-methyl-propyl]-amide;
1-Methyl-1H-imidazole-4-carboxylic acid (3-amino-propyl)-[(R)-1-(3-benzyl-7-fluoro-4-oxo-4H-chromen-2-yl)-2-methyl-propyl]-amide;
1-Methyl-1H-pyrrole-2-carboxylic acid (3-amino-propyl)-[(R)-1-(3-benzyl-7-fluoro-4-oxo-4H-chromen-2-yl)-2-methyl-propyl]-amide;
Benzo[c]isoxazole-3-carboxylic acid (3-amino-propyl)-[(R)-1-(3-benzyl-7-fluoro-4-oxo-4H-chromen-2-yl)-2-methyl-propyl]-amide; and
5-Methyl-isoxazole-3-carboxylic acid (3-amino-propyl)-[(R)-1-(3-benzyl-7-fluoro-4-oxo-4H-chromen-2-yl)-2-methyl-propyl]-amide, and
pharmaceutically acceptable salts thereof.

27. A pharmaceutical composition comprising
[a] an effective amount of a chromenone derivative chosen from compounds of Formula I 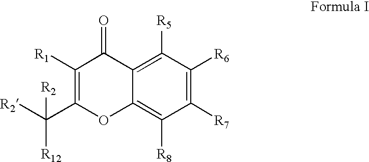
wherein:
R₁ is chosen from hydrogen, optionally substituted alkyl-, optionally substituted aryl-, optionally substituted aralkyl-, optionally substituted heteroaryl-, and optionally substituted heteroaralkyl-;
R₂ and R_{2′} are independently chosen from hydrogen, optionally substituted alkyl-, optionally substituted alkoxy, optionally substituted aryl-, optionally substituted aralkyl-, optionally substituted heteroaryl-, and optionally substituted heteroaralkyl-; or R₂ and R_{2′} taken together form an optionally substituted 3- to 7-membered ring;
R₁₂ is selected from the group consisting of optionally substituted imidazolyl, optionally substituted imidazolinyl, —NHR₄; —N(R₄)(COR₃); —N(R₄)(SO₂R₃ₐ); and —N(R₄)(CH₂R_{3b});
R₃ is chosen from hydrogen, optionally substituted alkyl-, optionally substituted aryl-, optionally substituted aralkyl-, optionally substituted heteroaryl-, optionally substituted heteroaralkyl-, R₁₅O— and R₁₇—NH—;
R₃ₐ is chosen from optionally substituted alkyl-, optionally substituted aryl-, optionally substituted aralkyl-, optionally substituted heteroaryl-, optionally substituted heteroaralkyl-, and R₁₇—NH—;
R_{3b} is chosen from hydrogen, optionally substituted alkyl-, optionally substituted aryl-, optionally substituted aralkyl-, optionally substituted heteroaryl-, and optionally substituted heteroaralkyl-;
R₄ is chosen from hydrogen, optionally substituted alkyl-, optionally substituted aryl-, optionally substituted aralkyl-, optionally substituted heterocyclyl-, and optionally substituted heteroaralkyl-;
R₅, R₆, R₇ and R₈ are independently chosen from hydrogen, acyl, optionally substituted alkyl-, optionally substituted alkoxy, halogen, hydroxyl, nitro, cyano, dialkylamino, alkylsulfonyl-, alkylsulfonamido-, alkylthio-, carboxyalkyl-, carboxamido-, aminocarbonyl-, optionally substituted aryl and optionally substituted heteroaryl-;
R₁₅ is chosen from optionally substituted alkyl-, optionally substituted aryl-, optionally substituted aralkyl-, optionally substituted heteroaryl-, and optionally substituted heteroaralkyl-; and
R₁₇ is hydrogen, optionally substituted alkyl-, optionally substituted aryl-, optionally substituted aralkyl-, optionally substituted heteroaryl-, or optionally substituted hetero-aralkyl, and
pharmaceutically acceptable salts thereof, and
[b] a diluent chosen from 5% mannitol injection, 0.9% sodium chloride injection and 5% dextrose injection.

28. An article of manufacture for use in connection with treating cancer in a human patient comprising a pharmaceutical composition of claim 27 contained within an infusion bag or a sterile evacuated glass bottle and instructions for the use of said pharmaceutical composition.
